(19) United States
(12) Patent Application Publication
(10) Pub. No.: US 2010/0196366 A1
(43) Pub. Date: Aug. 5, 2010

(54) Gefitnib Sensitivity-Related Gene Expression and Products and Methods Related Thereto

(76) Inventors: Paul A. Bunn, Evergreen, CO (US); Christopher D. Coldren, Denver, CO (US); Wilbur A. Franklin, Denver, CO (US); Mark W. Geraci, Aurora, CO (US); Barbara A. Helfrich, Lakewood, CO (US); Fred R. Hirsch, Denver, CO (US); Razvan Lapadat, Denver, CO (US); Michio Sugita, Centennial, CO (US); Samir E. Witta, Greenwood Village, CO (US)

Correspondence Address: SHERIDAN ROSS PC, 1560 BROADWAY, SUITE 1200, DENVER, CO, 80202, US

(21) Appl. No.: 12/670,052

(22) PCT Filed: Jul. 23, 2008
(86) PCT No.: PCT/US08/70930

§ 371 Date: Apr. 21, 2010

(30) Foreign Application Priority Data
Jul. 23, 2007 (US) 11/781946

(51) Int. Cl. A61K 39/395; C12Q 1/68; A61K 31/5377; A61K 31/517; A61K 31/497

(52) U.S. Cl. 424/133.1; 435/6; 514/234.5; 514/266.4; 514/252.18; 424/141.1

(57) ABSTRACT

Disclosed is the identification, provision and use of a panel of biomarkers that predict sensitivity or resistance to EGFR inhibitors, and products and processes related thereto. In one embodiment, a method is described for selecting a cancer patient who is predicted to benefit from therapeutic administration of an EGFR inhibitor, an agonist thereof, or a drug having substantially similar biological activity as EGFR inhibitor. Also described is a method to identify molecules that interact with the EGFR pathway to allow or enhance responsiveness to EGFR inhibitors, as well as a plurality of polynucleotides or antibodies for detection of the expression of genes that are indicative of sensitivity or resistance to EGFR inhibitors, an agonist thereof, or a drug having substantially similar biological activity as EGFR inhibitors. A method to identify a compound with the potential to enhance the efficacy of EGFR inhibitors is also described.

### CROSS-REFERENCE

This application is a continuation-in-part application of U.S. application Ser. No. 11/781,946, filed on Jul. 23, 2007, which is a continuation in part of U.S. application Ser. No. 10/587,052, filed Jul. 24, 2006, to which application we claim priority under 35 USC §120, which claimed priority to PCT/US2005/002325, filed Jan. 24, 2005, which claimed priority to U.S. Provisional Application No. 60/538,682, filed Jan. 23, 2004. Each of these applications are incorporated herein by reference in their entirety.

### FIELD OF THE INVENTION

This invention generally relates to methods to screen for patients that are predicted to benefit from therapeutic administration of gefitinib, as well as methods to identify compounds that interact with the epidermal growth factor receptor (EGFR) pathway to allow or enhance responsiveness to EGFR inhibitors, and products and methods related thereto.

### BACKGROUND OF THE INVENTION

Lung Cancer is the leading cause of death from cancer worldwide. Chemotherapy is the mainstay of treatment for lung cancer. However, less than a third of patients with advanced stages of non-small cell lung cancer (NSCLC) respond to the best two chemotherapy drug combinations. Therefore, novel agents that target cancer specific biological pathways are needed.

The epidermal growth factor receptor (EGFR) is one of the most appealing targets for novel therapies for cancer. EGFR plays a major role in transmitting stimuli that lead to proliferation, growth and survival of various cancer types, including, but not limited to, NSCLC. Ligand binding to the EGFR receptor leads to homo- or heterodimerization of EGFR with other ErbB receptors. EGFR is overexpressed in a large proportion of invasive NSCLC and in premalignant bronchial lesions/Bronchioloalveolar carcinoma/(BAC), a subtype of non-small cell lung cancer, represents the major form of lung cancer in non-smoking females and is rising in frequency, and epidermal growth factor receptor (EGFR) is expressed with high frequency in BAC. Unfortunately, the response of BACs to conventional chemotherapy is poor. Activation of EGFR leads to simultaneous activation of several signaling cascades including the MAPK pathway, the protein kinase C (PKC) pathway and the PI(3)K-activated AKT pathway (FIG. 1). EGFR signaling translated in the nucleus leads to cancer cell proliferation and survival.

Targeted therapy against the EGFR receptor has produced response rates of 25-30% as first line treatment and 11-20% in 2nd and 3rd line settings (e.g., chemo-refractory advanced stage NSCLC). For example, in phase II clinical trials, 11-20% of patients with chemo-refractory advanced stage NSCLC responded to treatment with the EGFR tyrosine kinase inhibitor gefitinib (commercially available as Iressa®, ZD1839). A trial evaluating the activity of the EGFR inhibitor, erlotinib (Tarceva®, OSI-774) has been completed and the results will be reported in the near future. A retrospective analysis of 140 patients responding to treatment with gefitinib revealed that the presence of BAC features (p=0.005) and being a never smoker (p=0.007) were the only independent 5 predictors of response to gefitinib. These data suggest that EGFR inhibitor therapy is more active in BAC and in non-smokers.

However, currently, there are no selection criteria for determining which NSCLC patients will benefit from treatment with EGFR inhibitors such as gefitinib. Moreover, EGFR expression does not predict gefitinib sensitivity. Therefore, despite the correlation of tumor histology and smoking history with gefitinib response, it is of great importance to identify molecular molecules that influence gefitinib responsiveness, and to develop adjuvant treatments that enhance the response. To accomplish this goal, there is a need in the art to define critical aspects of EGFR signaling and to identify which molecules interact with the EGFR pathway to dictate responsiveness to EGFR inhibitors.

### SUMMARY OF THE INVENTION

One embodiment of the present invention relates to a method to select a cancer patient who is predicted to benefit from therapeutic administration of an EGFR inhibitor, an agonist thereof, or a drug having substantially similar biological activity as EGFR inhibitor. The method includes the steps of: (a) providing a sample of tumor cells from a patient to be tested; (b) detecting in the sample the expression of one or more genes chosen from a panel of genes whose expression has been correlated with sensitivity or resistance to an EGFR inhibitor; (c) comparing the level of expression of the gene or genes detected in the patient sample to a level of expression of the gene or genes that has been correlated with sensitivity or resistance to the EGFR inhibitor; and (d) selecting the patient as being predicted to benefit from therapeutic administration of the EGFR inhibitor, if the expression of the gene or genes in the patient's tumor cells is statistically more similar to the expression levels of the gene or genes that has been correlated with sensitivity to the EGFR inhibitor than to resistance to the EGFR inhibitor.

In one aspect, the panel of genes in (b) is identified by a method comprising: (a) providing a sample of cells that are sensitive or resistant to treatment with the EGFR inhibitor; (b) detecting the expression of at least one gene in the EGFR inhibitor-sensitive cells as compared to the level of expression of the gene or genes in the EGFR inhibitor-resistant cells; and (c) identifying a gene or genes having a level of expression in EGFR inhibitor-sensitive cells that is statistically significantly different than the level of expression of the gene or genes in EGFR inhibitor-resistant cells, as potentially being a molecule that interacts with the EGFR pathway to allow or enhance responsiveness to EGFR inhibitors.

In another aspect, the EGFR inhibitor is gefitinib. In this aspect, step (b) can include, in one embodiment, detecting in the sample the expression of one or more genes chosen from a gene comprising, or expressing a transcript comprising, a nucleic acid sequence selected from the group consisting of SEQ ID NOs: 1-197. Step (c) comprises comparing the level of expression of the gene or genes detected in the patient sample to a level of expression of the gene or genes that has been correlated with sensitivity or resistance to gefitinib. Step (d) comprises selecting the patient as being predicted to benefit from therapeutic administration of gefitinib, an agonist thereof, or a drug having substantially similar biological activity as gefitinib, if the expression of the gene or genes in the patient's tumor cells is statistically more similar to the expression levels of the gene or genes that has been correlated with sensitivity to gefitinib than to resistance to gefitinib.

In any of the embodiments above, the method can include detecting expression of at least two genes in (b), at least three genes in (b), at least four genes in (b), at least five genes in (b), at least 10 genes in (b), at least 25 genes in (b), at least 50 genes from in (b), at least 100 genes in (b), at least 150 genes in (b), or up to all of the genes in the panel of genes.

In one aspect of this method, expression of the gene or genes is detected by measuring amounts of transcripts of the gene in the tumor cells. In another aspect, expression of the gene or genes is detected by detecting hybridization of at least a portion of the gene or a transcript thereof to a nucleic acid molecule comprising a portion of the gene or a transcript thereof in a nucleic acid array. In another aspect, expression of the gene is detected by detecting the production of a protein encoded by the gene. In yet another aspect, the method includes detecting expression of at least one gene selected from the group consisting of: E-cadherin (represented by SEQ ID NO:3) and ErbB3 (represented by SEQ ID NO:15 or SEQ ID NO:133). For example, the method can include detecting expression of at least one gene selected from the group consisting of ZEB1 and SIP1.

In one aspect of this method, the method includes comparing the expression of the gene or genes to expression of the gene or genes in a cell from a non-cancerous cell of the same type. In another aspect, the method includes comparing the expression of the gene or genes to expression of the gene or genes in an autologous, non-cancerous cell from the 5 patient. In another aspect, the method includes comparing the expression of the gene or genes to expression of the gene or genes in a control cell that is resistant to the EGFR inhibitor. In yet another aspect, the method includes comparing the expression of the gene or genes to expression of the gene or genes in a control cell that is sensitive to the EGFR inhibitor. In another aspect, control expression levels of the gene or genes that has been correlated with sensitivity and/or resistance to the EGFR inhibitor has been predetermined.

Yet another embodiment of the present invention relates to a method to identify molecules that interact with the EGFR pathway to allow or enhance responsiveness to EGFR inhibitors. The method includes the steps of: (a) providing a sample of cells that are sensitive or resistant to treatment with gefitinib; (b) detecting the expression of at least one gene in the gefitinib-sensitive cells as compared to the level of expression of the gene or genes in the gefitinib-resistant cells; and (c) identifying a gene or genes having a level of expression in gefitinib-sensitive cells that is statistically significantly different than the level of expression of the gene or genes in gefitinib-resistant cells, as potentially being a molecule that interacts with the EGFR pathway to allow or enhance responsiveness to EGFR inhibitors.

Another embodiment of the present invention relates to a plurality of polynucleotides for the detection of the expression of genes that are indicative of sensitivity or resistance to gefitinib, an agonist thereof, or a drug having substantially similar biological activity as gefitinib. The plurality of polynucleotides consists of at least two polynucleotides, wherein each polynucleotide is at least 5 nucleotides in length, and wherein each polynucleotide is complementary to an RNA transcript, or nucleotide derived therefrom, of a gene that is regulated differently in gefitinib-sensitive tumor cells as compared to gefitinib-resistant cells. In one aspect, each polynucleotide is complementary to an RNA transcript, or a polynucleotide derived therefrom, of a gene comprising, or expressing a transcript comprising, a nucleic acid sequence selected from the group consisting of SEQ ID NOs: 1-197. In another aspect, the plurality of polynucleotides comprises polynucleotides that are complementary to an RNA transcript, or a nucleotide derived therefrom, of at least two genes comprising, or expressing a transcript comprising, a nucleic acid sequence selected from the group consisting of SEQ ID NOs: 1-197. In another aspect, the plurality of polynucleotides comprises polynucleotides that are complementary to an RNA transcript, or a nucleotide derived 5 therefrom, of at least five genes, at least 10 genes, at least 25 genes, at least 50 genes, at least 100 genes, at least 150 genes, or up to all of the genes, comprising, or expressing a transcript comprising, a nucleic acid sequence selected from the group consisting of SEQ ID NOs: 1-197. In one aspect, the polynucleotide probes are immobilized on a substrate. In another aspect, the polynucleotide probes are hybridizable array elements in a microarray. In yet another aspect, the polynucleotide probes are conjugated to detectable markers.

Yet another embodiment of the present invention relates to a plurality of antibodies, antigen binding fragments thereof, or antigen binding peptides, for the detection of the expression of genes that are indicative of sensitivity or resistance to gefitinib, an agonist thereof, or a drug having substantially similar biological activity as gefitinib. The plurality of antibodies, antigen binding fragments thereof, or antigen binding peptides consists of at least two antibodies, antigen binding fragments thereof, or antigen binding peptides, each of which selectively binds to a protein encoded by a gene comprising, or expressing a transcript comprising, a nucleic acid sequence selected from the group consisting of SEQ ID NOs: 1-197.

Another embodiment of the present invention relates to a method to identify a compound with the potential to enhance the efficacy of EGFR inhibitors. The method includes the steps of: (a) contacting a test compound with a cell that expresses at least one gene, wherein said gene is selected from any one of the genes comprising, or expressing a transcript comprising, a nucleic acid sequence selected from the group consisting of SEQ ID NOs: 1-197; (b) identifying compounds selected from the group consisting of: (i) compounds that increase the expression or activity of the gene or genes in (a), or the proteins encoded thereby, that are correlated with sensitivity to gefitinib; and (ii) compounds that decrease the expression or activity of genes in (a), or the proteins encoded thereby, that are correlated with resistance to gefitinib. The compounds are identified as having the potential to enhance the efficacy of EGFR inhibitors. In one aspect of this embodiment, the cell expresses a gene encoding E-cadherin or ErbB3, and wherein step (b) comprises identifying compounds that increase the expression or activity of E-cadherin or ErbB3 or the gene encoding E-cadherin or ErbB3. In another aspect of this embodiment, the cell expresses a gene encoding ZEB1 and SIP1, wherein step (b) comprises identifying compounds that decrease the expression or activity ZEB1 or SIP1 or the gene encoding ZEB1 or SIP1.

Another embodiment of the present invention relates to a method to treat a patient with a cancer, comprising administering to the patient a therapeutic composition comprising a compound identified by the method described above.

Yet another embodiment of the present invention relates to a method to treat a patient with a cancer, comprising administering to the patient a therapeutic composition comprising a compound that upregulates the expression or activity of E-cadherin or ErbB3 or the gene encoding E-cadherin or ErbB3 in the tumor cells of the patient. Another embodiment of the present invention relates to a method to treat a patient with a cancer, comprising administering to the patient a therapeutic composition comprising a compound that downregulates the expression of ZEB1 or SIP1 or the gene encoding ZEB1 or SIP1 in the tumor cells of the patient.

Provided herein is a diagnostic method including the steps of providing a sample of cancer cells of epithelial origin from a patient to be tested, detecting the expression of at least one gene chosen from a panel of genes whose expression has been correlated with sensitivity or resistance to an antibody that binds EGFR, and comparing the level of expression of a gene detected in the patient sample to a level of expression a gene that has been correlated with sensitivity or resistance to the antibody that binds EGFR. In some embodiments, the gene or genes are chosen from E-cadherin, RAB25, integrin beta 6 (ITGB6), vimentin, ZEB1 and SIP1. In on embodiment, the gene is E-cadherin. In another embodiment, the gene is RAB25. In yet another embodiment, the gene is integrin beta 6. In still another embodiment, the gene is vimentin. In yet another embodiment, the gene is ZEB1. In another embodiment the gene is SIP1.

Also provided herein, including any of the aforementioned embodiments, are diagnostic methods including the steps of providing a sample of cancer cells of epithelial origin from a patient to be tested, detecting the expression of at least one gene chosen from a panel of genes whose expression has been correlated with sensitivity or resistance to an antibody that binds EGFR, and comparing the level of expression of a gene detected in the patient sample to a level of expression a gene that has been correlated with sensitivity or resistance to the antibody that binds EGFR, and where the antibody is cetuximab, panitumumab, nimotuzumab, or matuzumab. In one embodiment, the antibody is cetuximab. In yet another embodiment, the antibody is Panitumamab. In another embodiment, the antibody is nimotuzumab. In yet another embodiment, the antibody is matuzumab.

Provided herein, including any of the aforementioned embodiments, are diagnostic methods including the steps of providing a sample of cancer cells of epithelial origin from a patient to be tested, detecting the expression of at least one gene chosen from a panel of genes whose expression has been correlated with sensitivity or resistance to an antibody that binds EGFR, and comparing the level of expression of a gene detected in the patient sample to a level of expression a gene that has been correlated with sensitivity or resistance to the antibody that binds EGFR, where the cancer cells are selected from breast cancer cells, skin cancer cells, bladder cancer cells, colon cancer cells, prostate cancer cells, uterine cancer cells, cervical cancer cells, ovarian cancer cells, esophageal cancer cells, stomach cancer cells, gastrointestinal cancer cells, pancreatic cancer cells, laryngeal cancer cells, and lung cancer cells. In one embodiment, the cancer cells are from pancreatic cancer cells. In another embodiment, the cancer cells are from head and neck cancer cells. In yet another embodiment, the cancer cells are from breast cancer cells. In another embodiment, the cancer cells are from colon cancer cells.

Provided herein is a diagnostic method including the steps of providing a sample of cancer cells of epithelial origin from a patient to be tested, detecting the expression of at least one gene chosen from a panel of genes whose expression has been correlated with sensitivity or resistance to an antibody that binds EGFR, and comparing the level of expression of a gene detected in the patient sample to a level of expression a gene that has been correlated with sensitivity or resistance to the antibody that binds EGFR, and further selecting the patient as being predicted to benefit from therapeutic administration of the antibody that binds EGFR. In one embodiment, the expression of at least one gene in the patient's cancer cells is statistically more similar to the expression levels of at least one gene that has been correlated with sensitivity to the antibody that binds EGFR than to resistance to the antibody that binds EGFR. In another embodiment, the expression of at least one gene in the patient's cancer cells is statistically more similar to the expression levels of at least one gene that has been correlated with resistance to the antibody that binds EGFR than to resistance to the antibody that binds EGFR. In additional embodiments, the panel of genes in is identified by providing a sample of cells that are sensitive or resistant to treatment with the antibody that binds EGFR, detecting the expression of at least one gene in the antibody-sensitive cells as compared to the level of expression of the gene or genes in the antibody-resistant cells, and identifying a gene or genes having a level of expression in the antibody-sensitive cells that is statistically significantly different than the level of expression of the gene or genes in the antibody-resistant cells. In still further embodiments, expression of the gene(s) is detected by measuring amounts of transcripts of the gene in the tumor cells, detecting hybridization of at least a portion of the gene or a transcript thereof to a nucleic acid molecule comprising a portion of the gene or a transcript thereof in a nucleic acid array, and/or detecting the production of a protein encoded by the gene.

Also provided herein is a method of detecting sensitivity of an epithelial-origin cancer to an antibody the binds EGFR by detecting in a sample of tumor cells from a patient to be tested, the expression of one or more genes selected from the group consisting of E-cadherin, RAB25, TCF8, integrin beta 6 (ITGB6), vimentin, ZEB1 and SIP1, comparing the level of expression of the one or more genes detected in the patient sample to a gene expression level of E-cadherin, RAB25, integrin beta 6 (ITGB6), vimentin, ZEB1 or SIP1 that has been correlated with sensitivity or resistance to an antibody that binds EGFR, and identifying the expression level of the one or more genes detected in the patient sample that are statistically more similar to the expression level of E-cadherin, RAB25, integrin beta 6 (ITGB6), vimentin, ZEB1 or SIP1 that has been correlated with sensitivity than to the expression levels that have been correlated with resistance. In one embodiment, the gene is E-cadherin. In another embodiment, the gene is RAB25. In yet another embodiment, the gene is integrin beta 6 (ITGB6). In still another embodiment, the gene is vimentin. In another embodiment, the gene is ZEB1. In another embodiment, the gene is SIP1. In still further embodiments, including any of the aforemention embodiments, the antibody is cetuximab, panitumumab, nimotuzumab, or matuzumab. In still further embodiments, the cancer is breast cancer, skin cancer, bladder cancer, colon cancer, gastro-intestinal cancer, prostate cancer, uterine cancer, cervical cancer, ovarian cancer, esophageal cancer, stomach cancer, laryngeal cancer, or lung cancer. In yet another embodiment, the cancer is breast cancer. In another embodiment, the cancer is colon cancer. In another embodiment, the cancer is pancreatic cancer. In another embodiment, the cancer is a head and neck cancer.

Also provided herein are kits including reagents for the detection of expression levels that have been correlated with sensitivity or resistance to an EGFR inhibitor of one or more genes such as E-cadherin, RAB25, integrin beta 6, vimentin, ZEB1 and SIP1 in a sample of cancer cells. In some embodiments, the kits also include a compilation having E-cadherin, RAB25, integrin beta 6, vimentin, ZEB1 or SIP1 expression levels that have been correlated with sensitivity or resistance to an EGFR inhibitor. In one embodiment, the gene is E-cadherin. In one embodiment, the gene is ZEB1. In one embodiment, the gene is SIP1. In one embodiment, the gene is RAB25. In yet another embodiment, the gene is integrin beta 6. In another embodiment, the gene is vimentin.

Provided herein are methods of treating cancer in a patient by detecting the expression levels of one or more genes selected from E-cadherin, RAB25, integrin beta 6, vimentin, ZEB1 and SIP1 and administering an EGFR inhibitor. In one embodiment, the gene is E-cadherin. In one embodiment, the gene is ZEB1. In one embodiment, the gene is SIP1. In one embodiment, the gene is RAB25. In yet another embodiment, the gene is integrin beta 6. In another embodiment, the gene is vimentin. In additional embodiments, the EGFR inhibitor is selected from gefitinib, erlotinib, imatinib, lapatinib, and semazinib. In one embodiment, the EGFR inhibitor is gefitinib. In another embodiment, the EGFR inhibitor is erlotinib. In one embodiment, the EGFR inhibitor is imatinib. In one embodiment, the EGFR inhibitor is lapatinib. In one embodiment, the EGFR inhibitor is semazinib. In still further embodiments, the EGFR inhibitor is selected from cetuximab, panitumumab, nimotuzumab, and metuzumab. In one embodiment, the EGFR inhibitor is cetuximab. In another embodiment, the EGFR inhibitor is panitumumab. In yet another embodiment, the EGFR inhibitor is nimotuzumab. In one embodiment, the EGFR inhibitor is metuzumab.

Also provided herein are methods of treating cancer via upregulating E-cadherin in a cancer cell by administering to the patient at least one ZEB1 inhibitor and administering to the patient and EGFR inhibitor.

Further provided herein are methods of treating cancer in a patient via upregulating E-cadherin in a cancer cell by administering to the patient at least one SIP1 inhibitor and administering to the patient and EGFR inhibitor.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE FIGURES OF THE INVENTION

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic diagram showing the activation of signaling cascades from EGFR.

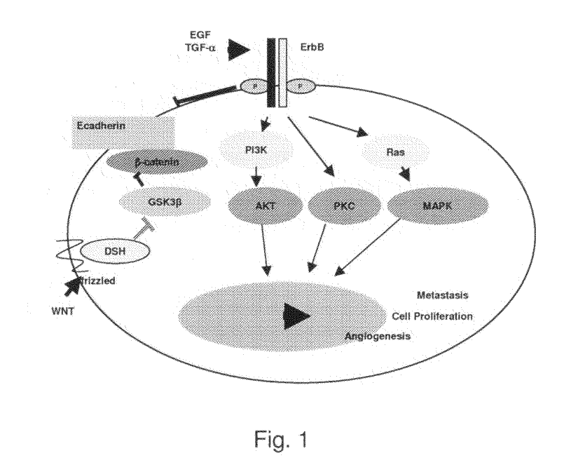

FIG. 2 is a schematic diagram showing E-cadherin regulation.

FIG. 3 is a digital image showing the expression of EGFR and phosphorylated EGFR in NSCLC cell lines.

FIG. 4 is a digital image showing that ZD1839 downregulates pEGFR in sensitive NSCLC cell lines.

FIG. 5 is a line graph showing the effects of gefitinib on A549 NSCLC xenografts.

FIG. 6 is a bar graph showing the expression of E-cadherin in NSCLC cell lines using GeneSpring analysis of microarrays.

FIG. 7 is a digital image showing Western blot analysis of E-cadherin expression in NSCLC cell lines.

FIG. 8 is a bar graph showing real time RT-PCR analysis of ZEB1 and SIP1 expression in NSCLC cell lines.

FIG. 9 is a schematic drawing showing the use of siRNA to silence the E-cadherin transcriptional repressors, SIP1 and ZEB1 to determine the effect on NSCLC cell line responses to ZD1839.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention generally relates to the identification, provision and use of a panel of biomarkers that predict sensitivity or resistance to gefitinib and other EGFR inhibitors, and products and processes related thereto. Specifically, the present inventors have used various cancer cell lines, including NSCLC cell lines, with varying sensitivity to the EGFR inhibitor, gefitinib, to define the novel panel of biomarkers as described herein. In order to identify a marker panel that could be used for selection of patients, including but not limited to NSCLC patients, who will respond to gefitinib treatment, the inventors undertook preclinical in vitro studies using various cell lines, including NSCLC cell lines. Based on the therapeutic response to gefitinib by using the IC₅₀ definition (i.e., the concentration of agent needed to kill 50% of the tumor cells in a cell culture), the present inventors have classified the cell lines as sensitive (IC₅₀<1 μM), resistant (IC₅₀>10 μM), or having intermediate sensitivity (1 μM<IC₅₀<10 μM) to gefitinib. The cell lines were characterized by gene microarray analysis (Affymetrix™ microarray Human Genome U133 set, 39,000 genes). By comparing the gene microarray results from sensitive and resistant cell lines, the inventors have identified a panel of genes that can discriminate between sensitive and resistant cell lines. These biomarkers (i.e., the genes identified) will be of great clinical significance in selecting patients/human tumors (including NSCLC patients) which will respond to this agent. The biomarkers identified by the present invention, and their expression levels in gefitinib sensitive and resistant cells, are listed in Table 1, and the nucleotide sequences representing such biomarkers are represented herein by SEQ ID NOs: 1-197. The nucleic acid sequences represented by SEQ ID NOs: 1-197 include transcripts or nucleotides derived therefrom (e.g., cDNA) expressed by the gene biomarkers in Table 1. It is to be understood that the present invention expressly covers additional genes that can be elucidated using substantially the same techniques used to identify the genes in Table 1 and that any of such additional genes can be used in the methods and products described herein for the genes and probe sets in Table 1. Any reference to database Accession numbers or other information regarding the genes and probe sets in Table 1 is hereby incorporated by reference in its entirety. For each biomarker listed in Table 1, the following information is provided: (1) the probe set ID number given by Affymetrix™ for the set of features on the array representing the indicated gene; (2) the parametric p-value, indicating the statistical significance of that individual gene expression difference; (3) the mean intensity of expression of each gene in a gefitinib-sensitive and a gefitinib-resistant cell line; (4) the HUGO-approved symbol for the gene, where one exists; (5) the sequence identifier representing a nucleotide sequence found in or transcribed by the gene; and (6) the name or title of the gene, where one is given. It is noted that sometimes two probe sets in Table 1 will refer to a single gene, and these duplications have been maintained because they are believed to reflect 5 different splice variants of that gene. In such a case, the associated sequence files will reflect the different splicotypes for that gene. The genes in Table 1 have been sorted by their parametric p-value to indicate the genes that are most highly regulated by gefitinib first.

In addition, the present invention will also be useful for the validation in other studies of the clinical significance of many of the specific biomarkers described herein, as well as the identification of preferred biomarker profiles, highly sensitive biomarkers, and targets for the design of novel therapeutic products and strategies. The biomarkers described herein are particularly useful in clinical practice to select the patients who will benefit most from EGFR inhibitor treatment, and in specific embodiments, from cetuximab treatment, panitumumab treatment, nimotuzumab treatment, matuzumab treatment, gefitinib treatment, erlotinib treatment, and/or lapatinib treatment.

The present inventors have already used the biomarkers described herein to identify specific targets for the further development of diagnostic and therapeutic approaches used in cancer, and these studies are described in detail in the Examples. For example, E-cadherin is a calcium-dependent epithelial cell adhesion molecule that plays an important role in tumor invasiveness and metastatic potential. Reduced E-cadherin expression is associated with tumor cell dedifferentiation, advanced stage and reduced survival in patients with NSCLC. Using Western blot analysis, E-cadherin was expressed in three cell lines highly sensitive to gefitinib and its expression was lacking in six gefitinib resistant cell lines tested. Real-time RT-PCR was used to evaluate the gene expression pattern in 11 NSCLC cell lines and compared to gene expression in normal bronchial epithelium. E-cadherin expression was elevated in cell lines sensitive to gefitinib and downregulated in the resistant cell lines as compared to the normal bronchial epithelium. The expression of E-cadherin is regulated by zinc finger inhibitory proteins by the recruitment of histone deacetylases (HDAC). Using real-time RT-PCR, the expression of the two zinc-finger transcription factors, δEF1/ZEB1 and SIP1/ZEB2, involved in E-cadherin repression was evaluated. Results showed that ZEB1 was expressed in gefitinib resistant cell lines and its expression was lacking in gefitinib sensitive cell lines. The present inventors have also found that δEF1/ZEB1 and SIP1/ZEB2 may regulate Her3, which is an EGFR heterodimer. These data indicate that the expression of ZEB1 may predict resistance to EGFR tyrosine kinase inhibitors and future studies directed at modulating the regulation of E-cadherin expression are expected to enhance the activity of EGFR inhibitors in various cancers, including NSCLC.

Epithelial-to-Mesenchymal Transition (EMT) refers to the process whereby a cell with a gene expression/phenotype characteristic of epithelial cell (i.e., expressing specific proteins, factors, and molecules) changes or alters the genes or their level of expression which results in a change in the phenotype of the cell as exhibited by the alteration or change in the genes expressed.

Epithelial and mesenchymal cells represent distinct lineages, each with a unique gene expression profile that imparts attributes specific to each cell type. Turning an epithelial cell into a mesenchymal cell requires alterations in morphology, cellular architecture, adhesion and migration capacity. Advanced tumor cells frequently exhibit a conspicuous down-regulation of epithelial markers and a loss of intercellular junctions, resulting in a loss of epithelial polarity and reduced intercellular adhesion. The loss of epithelial features is often accompanied by increased cell motility and expression of mesenchymal genes. EMT includes loss of contact inhibition, altered growth control, and enhanced invasiveness (Christiansen and Rajasekaran, Cancer Res., 66(17): 8319-8326 (2006); and Thiery et al., Curr. Opin. Cell. Biol., 15: 740-6 (2003)). Molecular and morphologic features indicative of EMT correlate with poor histologic differentiation, destruction of tissue integrity, and metastasis. EMT provides mechanisms for epithelial cells to overcome the physical constraints imposed on them by intercellular junctions and adopt a motile phenotype (Burdsal et al. Development, 118:829-44 (1993); and Nieto et al., Mech, Dev., 105:27-35 (2001)).

Commonly used molecular markers for EMT include increased expression of N-cadherin and vimentin, nuclear localization of β-catenin, and increased production of the transcription factors such as Snail1 (Snail), Snail2 (Slug), Twist, EF1/ZEB1, SIP1/ZEB2, and/or E47 that inhibit E-cadherin production. Phenotypic markers for EMT include, but are not limited to, an increased capacity for migration and three-dimensional invasion, as well as resistance to apoptosis. These markers have further been correlated with induction of EMT and an association with cancerous phenotypes. By way of example, loss of E-cadherin expression has been correlated with the transition of a pancreatic cancer from epithelial to mesenchymal. (Winter et al., Clin Cancer Res., 14(2): 412-418 (2008)).

The occurrence of EMT during tumor progression allows tumor cells to acquire the capacity to infiltrate surrounding tissue and to ultimately metastasize to distant sites. Changes in gene expression within tumor cells can indicate a progression from epithelial or epithelial-like gene expression pattern to a mesenchymal or mesenchymal-like gene expression pattern. By way of example, the identification of loss of E-cadherin is correlated with metastatic carcinoma as well as resistance to cancer therapies such as EGFR inhibitors. Analysis of many different types of cancer reveals that circulating tumor cells, or those found as micrometastases, evidence mesenchymal conversion based on changes of expression in a set of markers. These markers include, but are not limited to, EGFR, E-cadherin, ErbB3, RAB25, integrin beta 6, cadherin-2, fibroblast growth factor binding protein 1, distal-less homeo box 1, ZEB1 (transcription factor 8), SIP1, and vimentin.

By way of example, an epithelial-like gene expression profile includes expression, or increase expression of genes such as E-cadherin, ErbB3, or EGFR. An epithelial-like gene expression profile can include the expression of one or more of these genes, at least two, or at least three of these genes.

As with the previously described therapy-resistant cancers, the expression levels of E-cadherin, ErbB3, RAB25, integrin beta 6, cadherin-2, fibroblast growth factor binding protein 1, distal-less homeo box 1, ZEB1 (transcription factor 8), SIP1, TGF-β, FOXC2, GSK-3β, Smad-3, Pez, Snail1, Snail2, ILK, and vimentin represent genes that are common to EMT characteristics as well as with those epithelial-based tumor cells/cancers that develop resistance to their respective therapies.

In addition to the aforementioned genes and cancers, the expression levels of E-cadherin and vimentin can be correlated with sensitivity and/or resistance to tyrosine kinase EGFR inhibitors in head and neck cancer as well as non-small cell lung cancer. (Frederick et al., Mol. Cancer. Ther., 6(6): 1683-1691 (2006)). Similarly, HER-3 expression correlates with sensitivity or resistance to an EGFR inhibitor (erlotinib) in pancreatic tumor cell lines as well as colorectal tumor cell lines. (Buck et al., Mol. Cancer. Ther. 5(8): 2051-2059 (2006)). Furthermore, sensitivity and/or resistance of urothelial cancer to EGFR-antibody therapy is correlated with the expression levels of HER-4, E-cadherin, β-catenin, and PDGF-β. (Black et al., Clin. Cancer. Res., 14(5): 1478-1486 (2008)). Finally, multiple genes associated with the HER pathway, including E-cadherin, correlated with sensitivity and/or resistance to gefitinib in non-small cell lung cell cancer cell lines. (Coldren et al., Mol. Cancer. Res. 4(8): 521-528 (2006)).

In one non-limiting example, the present invention also relates to protein profiles which can discriminate between sensitive and resistant NSCLC tumors. Additional compounds may be screened for activity and/or efficacy in treating various other cancers. Similarly, biomarkers related to the sensitivity or resistance of a cancer to a given compound of can be screened. Furthermore, additional cancer types can be screened with the methods described herein.

Prior to the present invention, to the best of the present inventors' knowledge, no single marker, or marker panel, has been demonstrated to be useful for selection of lung cancer patients who will benefit from EGFR inhibitors, and particularly, gefitinib, treatment. Nor are there any such markers (related to EGFR inhibitors) identified for other types of cancer.

Accordingly, in one example using the gene expression profiles disclosed in Table 1 for gefitinib-sensitive and resistant cells, one can rapidly, effectively and efficiently screen patients/human tumors for a level of sensitivity or resistance to gefitinib and also to other EGFR inhibitors having biological activity substantially similar to gefitinib (i.e., drugs having similar activities, gefitinib agonists and other derivatives). The results will allow for the identification of tumors/patients that are likely to benefit from administration of the drug and therefore, the genes are used to enhance the ability of the clinician to develop prognosis and treatment protocols for the individual patient. In addition, genes identified in Table 1 can be further validated as targets and then used in assays to identify therapeutic reagents useful for regulating the expression or activity of the target in a manner that improves sensitivity of a cell to gefitinib or analogs thereof. The knowledge provided from the expression profile of genes described herein and the identification additional genes using similar methods can also be used to identify the molecular mechanisms of EGFR inhibition, such knowledge being useful for the further development of new therapies and even analogs of gefitinib or other EGFR inhibitors with improved efficacies in cancer treatment. Moreover, given the knowledge of these genes, one can produce novel combinations of polynucleotides and/or antibodies and/or peptides for use in the various assays, diagnostic and/or therapeutic approaches described herein.

The present invention is also illustrative of methods by which patients can be evaluated for predicted sensitivity or resistance to EGFR inhibitors other than gefitinib, and of methods of identifying additional genes and gene panels that are regulated differentially by cells that are sensitive to or resistant to gefitinib or other EGFR inhibitors. Such genes and panels of genes can then be used in the assays and methods described herein and as targets useful for the development of novel EGFR inhibitors and therapeutic formulations. In one embodiment, the gene or genes whose expression is detected is selected from among E-cadherin, Erb3, Her3, vimentin, cyclin D3, cyclin D1, EGFR, and any combination thereof. In yet another embodiment, the gene or genes whose expression is detected is selected from E-cadherin, ErbB3, RAB25, integrin beta 6, cadherin-2, fibroblast growth factor binding protein 1, distal-less homeo box 1, ZEB1 (transcription factor 8), and SIP1. In one embodiment, the gene is E-cadherin. In yet another embodiment, the gene is RAB25. In yet another embodiment, the gene is integrin beta 6. In yet another embodiment, the gene is ZEB1. In yet another embodiment, the gene is SIP1. In another embodiment, the gene is vimentin. It will be understood that any of the genes to be detected as described herein can be correlated with any of the EGFR inhibitors also described herein in more detail. It is understood that the gene or genes described herein are inclusive of allelic variant isoforms, synthetic nucleic acids, nucleic acid isolated from tissue and cells, and modified forms thereof. It is also understood that the genes described herein are also known to exist in various forms, including variants and mutants, and are contemplated herein. The gene or genes described herein further include nucleic acid sequences having at least 85% identity with the gene to be detected and are included within the invention and embodiments described herein. In some embodiments, the % identity is 90%. In some embodiments, the % identity is 95%. In yet other embodiments, the % identity is at least 99%.

In addition to gefitinib, various tyrosine-kinase inhibitors, including but not limited to EGFR inhibitors, are contemplated herein. Currently there are two main classes of EGFR inhibitors: anti-EGFR family tyrosine kinase inhibitors (small molecules) and anti-EGFR monoclonal antibodies. Both categories are contemplated within the meaning of EGFR inhibitor used herein. Examples of small molecules include EGFR-specific and reversible inhibitors such as, for example, gefitinib (IRESSA®, ZD1839), erlotinib (TARCEVA®, OSI-774, CP-358), or PKI-166; EGFR-specific and irreversible inhibitors, such as EKI-569; a PAN-HER (human EGF receptor family) reversible inhibitor, such as GW2016 (targets both EGFR and Her2/neu); and a PAN-HER irreversible inhibitor, such as CI-1033 (4-anilinoquinazoline).

Further examples of tyrosine kinase inhibitors and EGFR antagonists include, but are not limited to, small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Additional small molecule EGFR antagonists include, but are not limited to, PD 183805 (CI 1033, 2-propenamide, N-[4-[(3-chloro-4-fluorophenyl)amino]-7-[3-(4-morpholinyl)propoxy]-6-quin-azolinyl]-, dihydrochloride, Pfizer Inc.); ZM 105180 ((6-amino-4-(3-methylphenyl-amino)-quinazoline, Zeneca); BIBX-1382 (N-8-(3-chloro-4-fluoro-phenyl)-N-2-(1-methyl-piperidin-4-yl)-pyrimido[5,-4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-44-dimethylamino)-2-butenamide) (Wyeth); Imatinib; STI-571; LFM-A13; PD153035; Piceatannol; PP1, Lapatinib (Tykerb®, GW572016, GlaxoSmithKline); AEE788; SU4132; SU6656; Semazanib (semaxanib); SU6668, ZD6126 AG1478 (Sugen); and AG1571 (SU 5271; Sugen). Further examples of EGFR and HER family antagonists or inhibitors will be known in the art and are also contemplated herein.

Examples of monoclonal antibodies and antibody variants, fusions, derivatives, and fragments thereof include C225 (CETUXIMAB; ERBITUX®), ABX-EGF (human) (Abgenics, San Francisco, Calif.), EMD-72000 (humanized), h-R3 (humanized), and MDX-447 (bi-specific, EGFR-CK64); MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or panitumumab (see WO98/50433, Abgenix/Amgen); EMD 55900 (Stragliotto et al. Eur. J. Cancer 32A:636-640 (1996); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; and mAb 806 or humanized mAb 806 (Johns et al., J. Biol. Chem. 279(29):30375-30384 (2004)).

The anti-EGFR antibody may be conjugated with a cytotoxic agent, thus generating an immunoconjugate (see, e.g., EP659,439A2, Merck Patent GmbH). Additionally, fusion proteins, single chain antibodies, and fragments or variants thereof based upon the antibodies and epitope binding regions of the antibodies described above are also contemplated herein. The construction of such polypeptides, fusion proteins, and single chain antibodies is known in the art and can include, but is not limited to, conventional recombinant techniques.

The present invention provides methods by which patients can be evaluated for predicted sensitivity or resistance to antibody EGFR inhibitors, and of methods of identifying additional genes and gene panels that are regulated differentially by cells that are sensitive to or resistant to such antibody EGFR inhibitors. In some embodiments described herein, the genes detected are selected from E-cadherin, RAB25, integrin beta 6, vimentin, TC8, ZEB1 and SIP1 and the EGFR inhibitor is selected from cetuximab, panitumumab, nimotuzumab, and Metuzumab. In one embodiment, the EGFR inhibitor is cetuximab. In another embodiment, the inhibitor is panitumumab. In yet another embodiment, the EGFR inhibitor is nimotuzumab. In another embodiment, the EGFR inhibitor is Metuzumab. In a further embodiment, the gene is E-cadherin and the antibody is cetuximab. In another embodiment, the gene is E-cadherin and the antibody is panitumumab. In a further embodiment, the gene is E-cadherin and the antibody is nimotuzumab. In another embodiment, the gene is E-cadherin and the antibody is matuzumab.

In one embodiment, the gene is ZEB1 and the antibody is cetuximab. In one embodiment, the gene is ZEB1 and the antibody is panitumumab. In a further embodiment, the gene is ZEB1 and the antibody is nimotuzumab. In another embodiment, the gene is ZEB1 and the antibody is matuzumab.

In one embodiment, the gene is SIP1 and the antibody is cetuximab. In one embodiment, the gene is SIP1 and the antibody is panitumumab. In a further embodiment, the gene is SIP1 and the antibody is nimotuzumab. In another embodiment, the gene is SIP1 and the antibody is matuzumab.

It will be understood that sensitivity or resistance to any of the EGFR inhibitors described herein can be correlated with any of the genes also described herein.

The methods and embodiments of the invention described herein for the detection of genes correlated with EGFR sensitivity or resistance can further provide for methods of treatment based on such detection.

The transcription factors ZEB1 and SIP1 are also known to downregulate or inhibit the expression of E-cadherin. In one example, siRNA inhibitors of ZEB1 and/or SIP1 can upregulate expression of E-cadherin. The embodiments described herein can also be used in combination with methods of treating cancer in a patient by upregulating E-cadherin in a cancer cell via the administration of at least one ZEB1 or SIP1 inhibitor and the administration of an EGFR inhibitor. In one embodiment, a ZEB1 inhibitor is administered. In yet another embodiment, a SIP1 inhibitor is administered. In additional embodiments, a ZEB1 and a SIP1 inhibitor are administered. The ZEB1 and SIP1 inhibitors can be administered simultaneously or at different time points. In some embodiments, the administration of the ZEB1 or SIP1 inhibitor can be simultaneous with the EGFR inhibitor. In some embodiments the ZEB1 or SIP1 inhibitor can be administered prior to the EGFR inhibitor. In some embodiments, the ZEB1 or SIP1 inhibitor can be administered after the EGFR inhibitor. It will be understood that the embodiments described herein for the administration of ZEB1 and/or SIP1 inhibitors to upregulate E-cadherin expression are useful with any of the embodiments regarding the types of cancer to be treated also described herein.

In addition to the NSCLC described in several examples, the methods described herein can be used to identify biomarkers in numerous other cancer types. While NSCLC is used as an exemplary cancer, it will be understood in the art that other cancers are useful, and thus within the scope of the methods described herein. Such additional cancers include, but are not limited to, cancers that are epithelial malignancies (having epithelial origin), and particularly any cancers (tumors) that express EGFR. In one non-limiting example, provided herein is a method to identify a cancer that is resistant to EGFR inhibitors and in one aspect, the cancer is an epithelial malignancy that is resistant to EGFR inhibitors. In an EGFR inhibitor-resistant cancer, the cancer can include tumors (cancerous cells) with little or no gain in copy number (low/no gene amplification or polysomy), tumors that are low expressors of EGFR protein (in the lower 50% of an appropriate scoring protocol, as in PCT Publication No. WO 2005/117553), or especially a combination of low/no gain of EGFR gene and low/no expression of EGFR protein. EGFR-resistant cancers can also include tumors that have low/no gain in EGFR and are P-Akt positive, or tumors with EGFR gene amplification and/or polysomy, but that are P-Akt negative. EGFR-resistant cancers can also include tumors without mutations in EGFR that meet one or more of the other criteria for poor or non-responders as discussed above. Non-limiting examples of premalignant or precancerous cancers/tumors having epithelial origin include actinic keratoses, arsenic keratoses, xeroderma pigmentosum, Bowen's disease, leukoplakias, metaplasias, dysplasias and papillomas of mucous membranes, e.g. of the mouth, tongue, pharynx and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, precancerous changes of the bladder, e.g. metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), pancreactic cancer, colon cancer, gastrointestinal (GI) cancer, prostate cancer, uterine cancer, cervical cancer, ovarian cancer, esophageal cancer, stomach cancer, laryngeal cancer, head and neck cancer, and lung cancer.

In some embodiments, the cancer is selected from breast cancer, pancreatic cancer, colon cancer, and head and neck cancer. In one embodiment, the cancer is pancreatic cancer. In another embodiment, the cancer is colon cancer. In yet another embodiment, the cancer is head and neck cancer. In another embodiment, the cancer is breast cancer.

Provided herein is a method of selecting a cancer patient having a cancer of epithelial origin comprising providing a sample of the cancer from the patient, detecting the expression of one or more genes whose expression has been correlated with sensitivity or resistance to an EGFR inhibitor, comparing the level of expression of the gene or genes detected in the patient sample to a level of expression of the gene or genes that has been correlated with sensitivity or resistance to the EGFR inhibitor. In a further embodiment, a patient is selected as being predicted to benefit from administration of the EGFR inhibitor if the expression of the gene or genes is similar to the expression of the gene or genes that have been correlated with sensitivity to the EGFR inhibitor. Non-limiting examples of cancers having epithelial origin include breast cancer, skin cancer, bladder cancer, pancreatic cancer, colon cancer, prostate cancer, uterine cancer, cervical cancer, ovarian cancer, esophageal cancer, stomach cancer, gastrointestinal cancer (GI), pancreatic cancer, laryngeal cancer, head and neck cancer, and lung cancer. In one embodiment, the cancer is breast cancer. In another embodiment, the cancer is pancreatic cancer. In another embodiment, the cancer is colon cancer. In yet another embodiment, the cancer is head and neck cancer.

It will be understood that any of the cancers described herein can be assayed for gene expression of any of the genes described herein and further correlated with sensitivity or resistance to any of the EGFR inhibitors described herein.

Various definitions and aspects of the invention will be described below, but the invention is not limited to any specific embodiments that may be used for illustrative or exemplary purposes.

According to the present invention, in general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein can be referred to as amplification, overproduction, activation, enhancement, up-regulation or increased action of a protein.

According to the present invention, a “downstream gene” or “endpoint gene” is any gene, the expression of which is regulated (up or down) within a gefitinib sensitive or resistant cell. Selected sets of one, two, and preferably several or many of the genes (up to the number equivalent to all of the genes) of this invention can be used as end-points for rapid screening of patient cells for sensitivity or resistance to EGFR inhibitors such as gefitinib and for the other methods as described herein, including the identification of novel targets for the development of new cancer therapeutics.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either 5 enhanced, decreased, or substantially similar properties as compared to the naturally occurring protein or peptide. A homologue can include an agonist of a protein or an antagonist of a protein.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

An agonist can be any compound which is capable of mimicking, duplicating or approximating the biological activity of a naturally occurring or specified protein, for example, by associating with (e.g., binding to) or activating a protein (e.g., a receptor) to which the natural protein binds, so that activity that would be produced with the natural protein is stimulated, induced, increased, or enhanced. For example, an agonist can include, but is not limited to, a protein, compound, or an antibody that selectively binds to and activates or increases the activation of a receptor bound by the natural protein, other homologues of the natural protein, and any suitable product of drug design that is characterized by its ability to agonize (e.g., stimulate, induce, increase, enhance) the biological activity of a naturally occurring protein.

An antagonist refers to any compound or agent which is capable of acting in a manner that is antagonistic to (e.g., against, a reversal of, contrary to) the action of the natural agonist, for example by interacting with another protein or molecule in a manner that the biological activity of the naturally occurring protein or agonist is decreased (e.g., reduced, inhibited, blocked). Such a compound can include, but is not limited to, an antibody that selectively binds to and blocks access to a protein by its natural ligand, or reduces or inhibits the activity of a protein, a product of drug design that blocks the protein or reduces the biological activity of the protein, an anti-sense nucleic acid molecule that binds to a nucleic acid molecule encoding the protein and prevents expression of the protein, a ribozyme that binds to the RNA and prevents expression of 5 the protein, RNAi, an aptamer, and a soluble protein, which competes with a natural receptor or ligand.

Agonists and antagonists that are products of drug design can be produced using various methods known in the art. Various methods of drug design, useful to design mimetics or other compounds useful in the present invention are disclosed in Maulik et al., 1997, Molecular Biotechnology Therapeutic Applications and Strategies, Wiley-Liss, Inc., which is incorporated herein by reference in its entirety. An agonist or antagonist can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, natural or synthetic steroidal compounds, carbohydrates and/or natural or synthetic organic and non-steroidal molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

As used herein, the term “mimetic” is used to refer to any natural or synthetic compound, peptide, oligonucleotide, carbohydrate and/or natural or synthetic organic molecule that is able to mimic the biological action of a naturally occurring or known synthetic compound.

As used herein, the term “putative regulatory compound” or “putative regulatory ligand” refers to compounds having an unknown regulatory activity, at least with respect to the ability of such compounds to regulate the expression or biological activity of a gene or protein encoded thereby, or to regulate sensitivity or resistance to an EGFR inhibitor as encompassed by the present invention.

In accordance with the present invention, an isolated polynucleotide, which phrase can be used interchangeably with “an isolated nucleic acid molecule”, is a nucleic acid 5 molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. Polynucleotides useful in the plurality of polynucleotides of the present invention (described below) are typically a portion of a gene or transcript thereof of the present invention that is suitable for use, for example, as a hybridization probe or PCR primer for the identification of a full-length gene, a transcript thereof, or a polynucleotide derived from the gene or transcript (e.g., cDNA), in a given sample (e.g., a cell sample). An isolated nucleic acid molecule can include a gene or a portion of a gene (e.g., the regulatory region or promoter), for example, to produce a reporter construct according to the present invention. An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 51 and/or the 3′ end of the sequence) additional nucleic acids that do not normally flank the specified nucleic acid sequence in nature (i.e., heterologous sequences). Isolated nucleic acid molecules can include DNA, RNA (e.g., mRNA), or derivatives of either DNA or RNA (e.g., cDNA). Although the phrase “nucleic acid molecule” or “polynucleotide” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on the biological activity of the protein as described herein. Protein homologues (e.g., proteins encoded by nucleic acid homologues) have been discussed in detail above.

The minimum size of a nucleic acid molecule or polynucleotide of the present invention is a size sufficient to encode a protein having a desired biological activity, sufficient to form a probe or oligonucleotide primer that is capable of forming a stable hybrid with the complementary sequence of a nucleic acid molecule encoding the natural protein (e.g., under moderate, high or very high stringency conditions), or to otherwise be used as a target in an assay or in any therapeutic method discussed herein. If the polynucleotide is an oligonucleotide probe or primer, the size of the polynucleotide can be dependent on nucleic acid composition and percent homology or identity between the nucleic acid molecule and a complementary sequence as well as upon hybridization conditions per se (e.g., temperature, salt concentration, and formamide concentration). The minimum size of a polynucleotide that is used as an oligonucleotide probe or primer is at least about 5 nucleotides in length, and preferably ranges from about 5 to about 50 or about 500 nucleotides, including any length in between, in whole number increments (i.e., 5, 6, 7, 8, 9, 10, . . . 33, 34, . . . 256, 257, . . . 500), and more preferably from about 10 to about 40 nucleotides, and most preferably from about 15 to about 40 nucleotides in length. Additional polynucleotide probes can be about 500 nucleotides, about 750 nucleotide, about 1000 nucleotides, about 2000 nucleotides, about 5000 nucleotides, or about 10,000 nucleotides. In one aspect, the oligonucleotide primer or probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of a protein-encoding sequence or a nucleic acid sequence encoding a full-length protein.

An isolated protein, according to the present invention, is a protein (including a peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. An isolated protein useful as an antagonist or agonist according to the present invention can be isolated from its natural source, produced recombinantly or produced synthetically.

Smaller peptides useful as regulatory peptides are typically produced synthetically by methods well known to those of skill in the art.

According to the present invention, the phrase “selectively binds to” refers to the ability of an antibody, antigen binding fragment or binding partner (antigen binding peptide) to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

In some embodiments of the present invention, a compound is contacted with one or more nucleic acids or proteins. Such methods can include cell-based assays, or non-cell-based assay. In one embodiment, a target gene is expressed by a cell (i.e., a cell-based assay). In one embodiment, the conditions under which a cell expressing a target is contacted with a putative regulatory compound, such as by mixing, are conditions in which the expression or biological activity of the target (gene or protein encoded thereby) is not stimulated (activated) if essentially no regulatory compound is present. For example, such conditions include normal culture conditions in the absence of a known activating compound or other equivalent stimulus. The putative regulatory compound is then contacted with the cell. In this embodiment, the step of detecting is designed to indicate whether the putative regulatory compound alters the expression and/or biological activity of the gene or protein target as compared to in the absence of the putative regulatory compound (i.e., the background level).

In accordance with the present invention, a cell-based assay as described herein is conducted under conditions which are effective to screen for regulatory compounds or to profile gene expression as described in the methods of the present invention. Effective conditions include, but are not limited to, appropriate media, temperature, pH and oxygen conditions that permit the growth of the cell that expresses the receptor. An appropriate, or effective, medium is typically a solid or liquid medium comprising growth factors and assimilable carbon, nitrogen and phosphate sources, as well as appropriate salts, minerals, metals and other nutrients, such as vitamins. Culturing is carried out at a temperature, pH and oxygen content appropriate for the cell. Such culturing conditions are within the expertise of one of ordinary skill in the art.

Cells that are useful in the cell-based assays of the present invention include any cell that expresses a gene that is to be investigated as a target, or in the diagnostic assays described herein, any cell that is isolated from a patient, including normal or malignant (tumor) cells.

According to the present invention, the method includes the step of detecting the expression of at least one, and preferably more than one, and most preferably, several, of the genes that are regulated differently in EGFR inhibitor-sensitive versus EGFR inhibitor-resistant cells, and particularly, of the genes that have now been shown to be regulated differently in gefitinib-sensitive versus gefitinib-resistant cells, by the present inventors. As used herein, the term “expression”, when used in connection with detecting the expression of a gene, can refer to detecting transcription of the gene and/or to detecting translation of the gene. To detect expression of a gene refers to the act of actively determining whether a gene is expressed or not. This can include determining whether the gene expression is upregulated as compared to a control, downregulated as compared to a control, or unchanged as compared to a control. Therefore, the step of detecting expression does not require that expression of the gene actually is upregulated or downregulated, but rather, can also include detecting that the expression of the gene has not changed (i.e., detecting no expression of the gene or no change in expression of the gene).

The present method includes the step of detecting the expression of at least one gene set forth in Table 1. In a preferred embodiment, the step of detecting includes detecting the expression of at least 2 genes, and preferably at least 3 genes, and more preferably at least 4 genes, and more preferably at least 5 genes, and more preferably at least 6 genes, and more preferably at least 7 genes, and more preferably at least 8 genes, and more preferably at least 9 genes, and more preferably at least 10 genes, and more preferably at least 11 genes, and more preferably at least 12 genes, and more preferably at least 13 genes, and more preferably at least 14 genes, and more preferably at least 15 genes, and so on, in increments of one (i.e., 1, 2, 3, . . . 12, 13, . . . 56, 57, . . . 78, 79 . . . ), up to detecting expression of all of the genes disclosed herein in Table 1. For example, in one aspect of the invention, the expression of at least five genes is detected, and in another aspect, the expression of at least 10 genes is detected, and in another aspect, the expression of at least 25 genes is detected, and in another aspect, the expression of at least 50 genes is detected, and in another aspect, the expression of at least 100 genes is detected, and in another aspect, the expression of at least 150 genes is detected. Preferably, larger numbers of genes in Table 1 are detected, as this will increase the sensitivity of the detection method. Analysis of a number of genes greater than 1 can be accomplished simultaneously, sequentially, or cumulatively.

In another embodiment of the invention, detecting in the sample the expression of one or more genes chosen from a panel of genes whose expression has been correlated with sensitivity or resistance to an EGFR inhibitor. For example, such genes can be identified using the methods for identifying the genes whose expression is correlated with gefitinib-resistance or sensitivity as described herein. In one aspect, the panel of genes is identified by a method comprising: (a) providing a sample of cells that are sensitive or resistant to treatment with the EGFR inhibitor; (b) detecting the expression of at least one gene in the EGFR inhibitor-sensitive cells as compared to the level of expression of the gene or genes in the EGFR inhibitor-resistant cells; and (c) identifying a gene or genes having a level of expression in EGFR inhibitor-sensitive cells that is statistically significantly different than the level of expression of the gene or genes in EGFR inhibitor-resistant cells, as potentially being a molecule that interacts with the EGFR pathway to allow or enhance responsiveness to EGFR inhibitors. The present invention is not intended to be limited solely to the biomarkers listed in Table 1. Rather, the biomarkers of Table 1 illustrate various aspects of the invention that can now be achieved given the discoveries by the inventors. Therefore, although many of the embodiments below are discussed in terms gefitinib, it is to be understood that the methods of the invention can be extended to other EGFR inhibitors, and particularly to those that are similar in structure and/or function to gefitinib, including agonists of gefitinib.

In some embodiments, the first steps of the method to select a cancer patient that is predicted to benefit from therapeutic administration of an EGFR inhibitor, an agonist thereof, or a drug having substantially similar biological activity as EGFR inhibitor of the present invention, includes providing a patient sample (also called a test sample) and detecting in the sample the expression of a gene or genes. Suitable methods of obtaining a patient sample are known to a person of skill in the art. A patient sample can include any bodily fluid or tissue from a patient that may contain tumor cells or proteins of tumor cells. More specifically, according to the present invention, the term “test sample” or “patient sample” can be used generally to refer to a sample of any type which contains cells or products 5 that have been secreted from cells to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid.

A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch. A bodily fluid sample, like the tissue sample, contains the cells to be evaluated for marker expression or biological activity and/or may contain a soluble biomarker that is secreted by cells, and is a fluid obtained by any method suitable for the particular bodily fluid to be sampled. Bodily fluids suitable for sampling include, but are not limited to, blood, mucous, seminal fluid, saliva, breast milk, bile and urine.

In general, the sample type (i.e., cell, tissue or bodily fluid) is selected based on the accessibility and structure of the organ or tissue to be evaluated for tumor cell growth and/or on what type of cancer is to be evaluated. For example, if the organ/tissue to be evaluated is the breast, the sample can be a sample of epithelial cells from a biopsy (i.e., a cell sample) or a breast tissue sample from a biopsy (a tissue sample). The sample that is most useful in the present invention will be cells, tissues or bodily fluids isolated from a patient by a biopsy or surgery or routine laboratory fluid collection.

Once a sample is obtained from the patient, the sample is evaluated for the detection of the expression of the gene or genes that have been correlated with sensitivity or resistance to an EGFR inhibitor (e.g., gefitinib) of the present invention. For example, as discussed above, any one or more of the genes in Table 1 comprising or expressing a transcript comprising one of SEQ ID NOs: 1-197 are useful for detection in the present method.

In one aspect, it may be desirable to select those genes for detection that are particularly highly regulated in gefitinib-sensitive cells versus gefitinib-resistant cells in that they display the largest increases or decreases in expression levels. The detection of such genes can be advantageous because the endpoint may be more clear and require less quantitation. The relative expression levels of the genes identified in the present invention are listed in Table 1, and the genes are ranked in the Table. Therefore, one can easily select subsets of particularly highly regulated genes, or subsets of genes based on some other desired characteristic to provide a more robust, sensitive, or selective assay.

In one embodiment, one of skill in the art might choose to detect genes that exhibited a fold increase above background of at least 2. In another embodiment, one of skill in the art might choose to detect genes that exhibited a fold increase or decrease above background of at least 3, and in another embodiment at least 4, and in another embodiment at least 5, and in another embodiment at least 6, and in another embodiment at least 7, and in another embodiment at least 8, and in another embodiment at least 9, and in another embodiment at least 10 or higher fold changes. It is noted that fold increases or decreases are not typically compared from one gene to another, but with reference to the background level for that particular gene.

In one aspect of the method of the present invention, the step of detecting can include the detection of expression of one or more of the genes of this invention. Expression of transcripts and/or proteins is measured by any of a variety of known methods in the art. For RNA expression, methods include but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene.

In addition to general expression of a gene, the number of copies of a gene in a cancer cell/cells or tissue can be determined with nucleic acid probes to the genes. In one embodiment, Fluorescent in situ hybridization (FISH) can be used to detect the number of copies of a gene in a cancerous cell can be indicative of resistance or sensitivity to a compound. Established hybridization techniques such as FISH are contemplated herein. In one embodiment, the number of EGFR genes within a cancerous tissue or cell are detected using a FISH assay for the EGFR gene. Other non-limiting examples of genes that can be detected by FISH include E-cadherin and Her3. Additional genes for which knowledge of the extent of polysomy is desired will be known in the art and are contemplated herein.

Methods to measure protein expression levels generally include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry, as well as assays based on a property of the protein including but not limited to enzymatic activity or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O′Shannessy et al. Anal. Biochem. 212:457 (1993); Schuster et al., Nature 365:343 (1993)). Other suitable assays for measuring the binding of one protein to another include, for example, immunoassays such as enzyme linked immunoabsorbent assays (ELISA) and radioimmunoassays (RIA); or determination of binding by monitoring the change in the spectroscopic or optical properties of the proteins through fluorescence, UV absorption, circular dichroism, or nuclear magnetic resonance (NMR).

In one embodiment, immunohistochemistry (IHC) is used to determine the expression of a gene in a cancerous tissue or cell as an indicator of said cancer's sensitivity to EGFR inhibitors. Examples of genes whose expression is detected by IHC include EGFR, ErbB3, E-cadherein, and Her3. Other genes' expression as indicators of sensitivity and/or resistance to EGFR inhibitors can be determined as described herein.

Nucleic acid arrays are particularly useful for detecting the expression of the genes of the present invention. The production and application of high-density arrays in gene expression monitoring have been disclosed previously in, for example, WO 97/10365; WO 92/10588; U.S. Pat. No. 6,040,138; U.S. Pat. No. 5,445,934; or WO95/35505, all of which are incorporated herein by reference in their entireties. Also for examples of arrays, see Hacia et al. (1996) Nature Genetics 14:441-447; Lockhart et al. (1996) Nature Biotechnol. 14:1675-1680; and De Risi et al. (1996) Nature Genetics 14:457-460. In general, in an array, an oligonucleotide, a cDNA, or genomic DNA, that is a portion of a known gene occupies a known location on a substrate. A nucleic acid target sample is hybridized with an array of such oligonucleotides and then the amount of target nucleic acids hybridized to each probe in the array is quantified. One preferred quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization; however, it will be apparent to those of skill in the art that any similar systems or other effectively equivalent detection methods can also be used. In a particularly preferred embodiment, 5 one can use the knowledge of the genes described herein to design novel arrays of polynucleotides, cDNAs or genomic DNAs for screening methods described herein. Such novel pluralities of polynucleotides are contemplated to be a part of the present invention and are described in detail below.

Suitable nucleic acid samples for screening on an array contain transcripts of interest or nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. Preferably, the nucleic acids for screening are obtained from a homogenate of cells or tissues or other biological samples. Preferably, such sample is a total RNA preparation of a biological sample. More preferably in some embodiments, such a nucleic acid sample is the total mRNA isolated from a biological sample. Biological samples may be of any biological tissue or fluid or cells from any organism. Frequently the sample will be a “clinical sample” which is a sample derived from a patient, such as a lung tumor sample from a patient. Typical clinical samples include, but are not limited to, sputum, blood, blood cells (e.g., white cells), tissue or fine needle biopsy samples, urine, peritoneal fluid, and pleural fluid, or cells therefrom. Biological samples may also include sections of tissues, such as frozen sections or formalin fixed sections taken for histological purposes.

In one embodiment, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high-density 5 array may then include probes specific to the internal standard for quantification of the amplified nucleic acid. Other suitable amplification methods include, but are not limited to polymerase chain reaction (PCR) Innis, et al., PCR Protocols, A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87:1874 (1990)).

Nucleic acid hybridization simply involves contacting a probe and target nucleic acid under conditions where the probe and its complementary target can form stable hybrid duplexes through complementary base pairing. As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety. Nucleic acids that do not form hybrid duplexes are washed away from the hybridized nucleic acids and the hybridized nucleic acids can then be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

High stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). One of skill in the art can use the formulae in Meinkoth et al., 1984, Anal. Biochem. 138, 267-284 (incorporated herein by reference in its entirety) to calculate the appropriate hybridization and wash conditions to achieve these particular levels of nucleotide mismatch. Such conditions will vary, depending on whether DNA.-RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na+) at a temperature of between about 20° C. and about 35° C., more preferably, between about 28° C. and about 40° C., and even more preferably, between about 35° C. and about 45° C. In particular embodiments, stringent hybridization conditions for DNA:RNA hybrids include hybridization at an ionic strength of 6×SSC (0.9 M Na+) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 40%. Alternatively, Tm can be calculated empirically as set forth in Sambrook et al., supra, pages 9.31 to 9.62.

The hybridized nucleic acids are detected by detecting one or more labels attached to the sample nucleic acids. The labels may be incorporated by any of a number of means well known to those of skill in the art. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

In one aspect of the present method, in vitro cell based assays may be designed to screen for compounds that affect the regulation of genes at either the transcriptional or translational level. One, two or more promoters of the genes of this invention can be used to screen unknown compounds for activity on a given target. Promoters of the selected genes can be linked to any of several reporters (including but not limited to chloramphenicol acetyl transferase, or luciferase) that measure transcriptional read-out. The promoters can be tested as pure DNA, or as DNA bound to chromatin proteins.

In one aspect of the present method, the step of detecting can include detecting the expression of one or more genes of the invention in intact animals or tissues obtained from such animals. Mammalian (i.e. mouse, rat, monkey) or non-mammalian (i.e. chicken) species can be the test animals. Sample tissues from a patient can also be screened. The tissues to be surveyed can be either normal or malignant tissues. The presence and quantity of endogenous mRNA or protein expression of one or more of the genes of this invention can be measured in those tissues. The gene markers can be measured in tissues that are fresh, frozen, fixed or otherwise preserved. They can be measured in cytoplasmic or nuclear organ-, tissue- or cell-extracts; or in cell membranes including but not limited to plasma, cytoplasmic, mitochondrial, golgi or nuclear membranes; in the nuclear matrix; or in cellular organelles and their extracts including but not limited to ribosomes, nuclei, nucleoli, mitochondria, or golgi. Assays for endogenous expression of mRNAs or proteins encoded by the genes of this invention can be performed as described above. Alternatively, intact transgenic animals can be generated for screening for research or validation purposes.

Preferably, a gene identified as being upregulated or downregulated in a test cell according to the invention (including a sample tumor cell to be screened) is regulated in the same direction and to at least about 5%, and more preferably at least about 10%, and more preferably at least 20%, and more preferably at least 25%, and more preferably at least 30%, and more preferably at least 35%, and more preferably at least 40%, and more preferably at least 45%, and more preferably at least 50%, and preferably at least 55%, and more preferably at least 60%, and more preferably at least 65%, and more preferably at least 70%, and more preferably at least 75%, and more preferably at least 80%, and more preferably at least 85%, and more preferably at least 90%, and more preferably at least 95%, and more preferably of 100%, or any percentage change between 5% and higher in 1% increments (i.e., 5%, 6%, 7%, 8% . . . ), of the level of expression of the gene that is seen in established or confirmed EGFRi-sensitive or EGFRi-resistant cell lines. A gene identified as being upregulated or downregulated in a test cell according to the invention can also be regulated in the same direction and to a higher level than the level of expression of the gene that is seen in established or confirmed EGFRi-sensitive or EGFRi-resistant cells. In some embodiments, the cell lines are gefitinib-sensitive or gefitinib-resistant cells. In some embodiments, the cell lines are erlotinib-sensitive or erlotinib-resistant cells. In some embodiments, the cell lines are lapatinib-sensitive or lapatinib-resistant cells. In some embodiments, the cell lines are cetuximab-sensitive or cetuximab-resistant cells. In some embodiments, the cell lines are panitumumab-sensitive or panitumumab-resistant cells. In some embodiments, the cell lines are nimotuzumab-sensitive or nimotuzumab-resistant cells. In some embodiments, the cell lines are matuzumab-sensitive or matuzumab-resistant cells.

The values obtained from the test and/or control samples are statistically processed using any suitable method of statistical analysis to establish a suitable baseline level using methods standard in the art for establishing such values. Statistical significance according to the present invention should be at least p<0.05.

It will be appreciated by those of skill in the art that differences between the expression of genes in sensitive versus resistant cells may be small or large. Some small differences may be very reproducible and therefore nonetheless useful. For other purposes, large differences may be desirable for ease of detection of the activity. It will be therefore appreciated that the exact boundary between what is called a positive result and a negative result can shift, depending on the goal of the screening assay and the genes to be screened. For some assays it may be useful to set threshold levels of change. One of skill in the art can readily determine the criteria for screening of cells given the information provided herein.

The presence and quantity of each gene marker can be measured in primary tumors, metastatic tumors, locally recurring tumors, ductal carcinomas in situ, or other tumors. The markers can be measured in solid tumors that are fresh, frozen, fixed or otherwise preserved. They can be measured in cytoplasmic or nuclear tumor extracts; or in tumor membranes including but not limited to plasma, mitochondrial, golgi or nuclear membranes; in the nuclear matrix; or in tumor cell organelles and their extracts including 5 but not limited to ribosomes, nuclei, mitochondria, golgi.

The level of expression of the gene or genes detected in the test or patient sample of the invention is compared to a baseline or control level of expression of that gene. More specifically, according to the present invention, a “baseline level” is a control level of biomarker expression against which a test level of biomarker expression (i.e., in the test sample) can be compared. In the present invention, the control level of biomarker expression can be the expression level of the gene or genes in a control cell that is sensitive to the EGFR inhibitor, and/or the expression level of the gene or genes in a control cell that is resistant to the EGFR inhibitor. Other controls may also be included in the assay. In one embodiment, the control is established in an autologous control sample obtained from the patient. The autologous control sample can be a sample of isolated cells, a tissue sample or a bodily fluid sample, and is preferably a cell sample or tissue sample. According to the present invention, and as used in the art, the term “autologous” means that the sample is obtained from the same patient from which the sample to be evaluated is obtained. The control sample should be of or from the same cell type and preferably, the control sample is obtained from the same organ, tissue or bodily fluid as the sample to be evaluated, such that the control sample serves as the best possible baseline for the sample to be evaluated. In one embodiment, control expression levels of the gene or genes that have been correlated with sensitivity and/or resistance to the EGFR inhibitor has been predetermined, such as in Table 1. Such a form of stored information can include, for example, but is not limited to, a reference chart, listing or electronic file of gene expression levels and profiles for EGFR inhibitor sensitive and/or EGFR inhibitor resistant biomarker expression, or any other source of data regarding baseline biomarker expression that is useful in the method of the invention. Therefore, it can be determined, based on the control or baseline level of biomarker expression or biological activity, whether the expression level of a gene or genes in a patient sample is/are more statistically significantly similar to the baseline for EGFR resistance or EGFR sensitivity. A profile of individual gene markers, including a matrix of two or more markers, can be generated by one or more of the methods described above. According to the present invention, a profile of the genes in a tissue sample refers to a reporting of the expression level of a given gene from Table 1, and includes a classification of the gene with regard to how the gene is regulated in EGFRi-sensitive versus EGFRi-resistant cells. The data can be reported as raw data, and/or statistically analyzed by any of a variety of methods, and/or combined with any other prognostic marker(s). In some embodiments, the cells are gefitinib-sensitive or gefitinib-resistant cells. In some embodiments, the cells are gefitinib-sensitive or gefitinib-resistant cells. In some embodiments, the cells are erlotinib-sensitive or erlotinib-resistant cells. In some embodiments, the cells are lapatinib-sensitive or lapatinib-resistant cells. In some embodiments, the cells are cetuximab-sensitive or cetuximab-resistant cells. In some embodiments, the cells are panitumumab-sensitive or panitumumab-resistant cells. In some embodiments, the cells are nimotuzumab-sensitive or nimotuzumab-resistant cells. In some embodiments, the cells are matuzumab-sensitive or matuzumab-resistant cells.

Another embodiment of the present invention relates to a plurality of polynucleotides for the detection of the expression of genes as described herein. The plurality of polynucleotides consists of polynucleotides that are complementary to RNA transcripts, or nucleotides derived therefrom, of genes listed in Table 1 or otherwise identified as being useful according to the present invention (e.g., other genes correlated with sensitivity or resistance to gefitinib or another EGFR inhibitor), and is therefore distinguished from previously known nucleic acid arrays and primer sets. The plurality of polynucleotides within the above-limitation includes at least two or more polynucleotides that are complementary to RNA transcripts, or nucleotides derived therefrom, of one or more genes identified by the present inventors and listed in Table 1. Preferably, the plurality of polynucleotides is capable of detecting expression of at least two, and more preferably at least five, and more preferably at least 10, and more preferably at least 25, and more preferably at least 50, and more preferably at least 100, and more preferably at least 150, and more preferably all of the genes (or any number in between two and all of the genes, in whole increments) in a panel of genes correlated with EGFR inhibitor sensitivity and/or resistance, such as all of the genes listed in Table 1.

In one embodiment, it is contemplated that additional genes that are not regulated differently in gefitinib-sensitive versus gefitinib-resistant cells, or sensitive or resistant to any other EGFR inhibitor as described herein, can be added to the plurality of polynucleotides. Such genes would not be random genes, or large groups of unselected human genes, as are commercially available now, but rather, would be specifically selected to complement the sets of genes identified by the present invention. For example, one of skill in the art may wish to add to the above-described plurality of genes one or more genes that are of relevance because they are expressed by a particular tissue of interest (e.g., lung tissue), are associated with a particular disease or condition of interest (e.g., NSCLC), or are associated with a particular cell, tissue or body function (e.g., angiogenesis). The development of additional pluralities of polynucleotides (and antibodies, as disclosed below), which include both the above-described plurality and such additional selected polynucleotides, are explicitly contemplated by the present invention.

According to the present invention, a plurality of polynucleotides refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of polynucleotides, including at least 100, 500, 1000, 104, 105, or at least 106 or more polynucleotides.

In one embodiment, the polynucleotide probes are conjugated to detectable markers. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Preferably, the polynucleotide probes are immobilized on a substrate.

In one embodiment, the polynucleotide probes are hybridizable array elements in a microarray or high density array. Nucleic acid arrays are well known in the art and are described for use in comparing expression levels of particular genes of interest, for example, in U.S. Pat. No. 6,177,248, which is incorporated herein by reference in its entirety. Nucleic acid arrays are suitable for quantifying a small variations in expression levels of a gene in the presence of a large population of heterogeneous nucleic acids. Knowing the identity of the genes of the present invention, nucleic acid arrays can be fabricated either by de novo synthesis on a substrate or by spotting or transporting nucleic acid sequences onto specific locations of substrate. Nucleic acids are purified and/or isolated from biological materials, such as a bacterial plasmid containing a cloned segment of sequence of interest. It is noted that all of the genes identified by the present invention have been previously sequenced, at least in part, such that oligonucleotides suitable for the identification of such nucleic acids can be produced. The database accession number for each of the genes identified by the present inventors is provided in Table 1. Suitable nucleic acids are also produced by amplification of template, such as by polymerase chain reaction or in vitro transcription.

Synthesized oligonucleotide arrays are particularly preferred for this aspect of the invention. Oligonucleotide arrays have numerous advantages, as opposed to other methods, such as efficiency of production, reduced intra- and inter array variability, increased information content and high signal-to-noise ratio.

One of skill in the art will appreciate that an enormous number of array designs are suitable for the practice of this invention. An array will typically include a number of probes that specifically hybridize to the sequences of interest. In addition, in a preferred embodiment, the array will include one or more control probes. The high-density array chip includes “test probes.” Test probes could be oligonucleotides that range from about 5 to about 45 or 5 to about 500 nucleotides (including any whole number increment in between), more preferably from about 10 to about 40 nucleotides and most preferably from about 15 to about 40 nucleotides in length. In other particularly preferred embodiments the probes are 20 or 25 nucleotides in length. In another preferred embodiments, test probes are double or single strand DNA sequences. DNA sequences are isolated or cloned from natural sources or amplified from natural sources using natural nucleic acids as templates, or produced synthetically. These probes have sequences complementary to particular subsequences of the genes whose expression they are designed to detect. Thus, the test probes are capable of specifically hybridizing to the target nucleic acid they are to detect.

Another embodiment of the present invention relates to a plurality of antibodies, or antigen binding fragments thereof, for the detection of the expression of genes according to the present invention. The plurality of antibodies, or antigen binding fragments thereof, consists of antibodies, or antigen binding fragments thereof, that selectively bind to proteins encoded by genes described herein. According to the present invention, a plurality of antibodies, or antigen binding fragments thereof, refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of antibodies, or antigen binding fragments thereof, including at least 100, 500, or at least 1000 antibodies, or antigen binding fragments thereof.

The invention also extends to non-antibody polypeptides, sometimes referred to as binding partners or antigen binding peptides, that have been designed to bind specifically to, and either activate or inhibit as appropriate, a target protein. Examples of the design of such polypeptides, which possess a prescribed ligand specificity are given in Beste et al. (Proc. Natl. Acad. Sci. 96:1898-1903, 1999), incorporated herein by reference in its entirety.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (VL+CL domains) paired with the VH region and a portion of the Ch region (CHI domain). An Fab′ fragment corresponds to an Fab fragment with part of the hinge region attached to the CHI domain. An F(ab′)₂ fragment corresponds to two Fab′ fragments that are normally covalently linked to each other through a di-sulfide bond, typically in the hinge regions.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

Finally, any of the genes of this invention, or their RNA or protein products, can serve as targets for therapeutic strategies. For example, neutralizing antibodies could be directed against one of the protein products of a selected gene, expressed on the surface of a tumor cell. Alternatively, regulatory compounds that regulate (e.g., upregulate or downregulate) the expression and/or biological activity of a target gene (whether the product is intracellular, membrane or secreted), can be identified and/or designed using the genes described herein. For example, in one aspect, a method of using the genes described herein as a target includes the steps of: (a) contacting a test compound with a cell that expresses at least one gene, wherein said gene is selected from any one of the genes comprising, or expressing a transcript comprising, a nucleic acid sequence selected from the group consisting of SEQ ID NOs: 1-197; and (b) identifying compounds, wherein the compounds can include: (i) compounds that increase the expression or activity of the gene or genes in (a), or the proteins encoded thereby, that are correlated with sensitivity to at least one EGFRi; and (ii) compounds that decrease the expression or activity of genes in (a), or the proteins encoded thereby, that are correlated with resistance to the at least one EGFRi. The compounds are thereby identified as having the potential to enhance the efficacy of EGFR inhibitors. In some embodiments, the EGFRi is gefitinib. In some embodiments, the EGFRi is erlotinib. In some embodiments, the EGFRi is lapatinib. In some embodiments, the EGFRi is cetuximab. In some embodiments, the EGFRi is panitumumab. In some embodiments, the EGFRi is nimotuzumab. In some embodiments, the EGFRi is matuzumab.

The period of contact with the compound being tested can be varied depending on the result being measured, and can be determined by one of skill in the art. As used herein, the term “contact period” refers to the time period during which cells are in contact with the compound being tested. The term “incubation period” refers to the entire time during which cells are allowed to grow prior to evaluation, and can be inclusive of the contact period. Thus, the incubation period includes all of the contact period and may include a further time period during which the compound being tested is not present but during which expression of genes is allowed to continue prior to scoring. Methods to evaluate gene expression in a cell according to the present invention have been described previously herein.

If a suitable therapeutic compound is identified using the methods and genes of the present invention, a composition can be formulated. A composition, and particularly a therapeutic composition, of the present invention generally includes the therapeutic compound and a carrier, and preferably, a pharmaceutically acceptable carrier. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is preferably a rumor cell. In some embodiments, a suitable site for delivery is a site of inflammation, near the site of a tumor, or a site of any other disease or condition in which regulation of the genes identified herein can be beneficial. Preferred pharmaceutically acceptable carriers are capable of maintaining a compound, a protein, a peptide, nucleic acid molecule or mimetic (drag) according to the present invention in a form that, upon arrival of the compound, protein, peptide, nucleic acid molecule or mimetic at the cell target in a culture or in patient, the compound, protein, peptide, nucleic acid molecule or mimetic is capable of interacting with its target.

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into a patient or culture. As used herein, a controlled release formulation comprises a compound of the present invention (e.g., a protein (including homologues), a drug, an antibody, a nucleic acid molecule, or a mimetic) in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible). When the compound is a recombinant nucleic acid molecule, suitable delivery vehicles include, but are not limited to liposomes, viral vectors or other delivery vehicles, including ribozymes. Natural lipid-containing delivery vehicles include cells and cellular membranes. Artificial lipid-containing delivery vehicles include liposomes and micelles. A delivery vehicle of the present invention can be modified to target to a particular site in a patient, thereby targeting and making use of a compound of the present invention at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

A pharmaceutically acceptable carrier which is capable of targeting is herein referred to as a “delivery vehicle.” Delivery vehicles of the present invention are capable of delivering a composition of the present invention to a target site in a patient. A “target site” refers to a site in a patient to which one desires to deliver a composition. For example, a target site can be any cell which is targeted by direct injection or delivery using liposomes, viral vectors or other delivery vehicles, including ribozymes and antibodies. Examples of delivery vehicles include, but are not limited to, artificial and natural lipid-containing delivery vehicles, viral vectors, and ribozymes. Natural lipid-containing delivery vehicles include cells and cellular membranes. Artificial lipid-containing delivery vehicles include liposomes and micelles. A delivery vehicle of the present invention can be modified to target to a particular site in a subject, thereby targeting and making use of a compound of the present invention at that site. Suitable modifications include manipulating the chemical formula of the lipid portion of the delivery vehicle and/or introducing into the vehicle a compound capable of specifically 5 targeting a delivery vehicle to a preferred site, for example, a preferred cell type. Specifically, targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

Another preferred delivery vehicle comprises a viral vector. A viral vector includes an isolated nucleic acid molecule useful in the present invention, in which the nucleic acid molecules are packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

A composition can be delivered to a cell culture or patient by any suitable method. Selection of such a method will vary with the type of compound being administered or delivered (i.e., compound, protein, peptide, nucleic acid molecule, or mimetic), the mode of delivery (i.e., in vitro, in vivo, ex vivo) and the goal to be achieved by administration/delivery of the compound or composition. According to the present invention, an effective administration protocol (i.e., administering a composition in an effective manner) comprises suitable dose parameters and modes of administration that result in delivery of a composition to a desired site (i.e., to a desired cell) and/or in the desired regulatory event.

Administration routes include in vivo, in vitro and ex vivo routes. In vivo routes include, but are not limited to, oral, nasal, intratracheal injection, inhaled, transdermal, rectal, and parenteral routes. Preferred parenteral routes can include, but are not limited to, subcutaneous, intradermal, intravenous, intramuscular and intraperitoneal routes.

Intravenous, intraperitoneal, intradermal, subcutaneous and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art. Direct injection techniques are particularly useful for suppressing graft rejection by, for example, injecting the composition into the transplanted tissue, or for site-specific administration of a compound, such as at the site of a tumor. Ex vivo refers to performing part of the regulatory step outside of the patient, such as by transfecting a population of cells removed from a patient with a recombinant molecule comprising a nucleic acid sequence encoding a protein according to the present invention under conditions such that the recombinant molecule is subsequently expressed by the transfected cell, and returning the transfected cells to the patient. In vitro and ex vivo routes of administration of a composition to a culture of host cells can be accomplished by a method including, but not limited to, transfection, transformation, electroporation, microinjection, lipofection, adsorption, protoplast fusion, use of protein carrying agents, use of ion carrying agents, use of detergents for cell permeabilization, and simply mixing (e.g., combining) a compound in culture with a target cell.

In the method of the present invention, a therapeutic compound, as well as compositions comprising such compounds, can be administered to any organism, and particularly, to any member of the Vertebrate class, Mammalia, including, without limitation, primates, rodents, livestock and domestic pets. Livestock include mammals to be consumed or that produce useful products (e.g., sheep for wool production). Preferred mammals to protect include humans. Typically, it is desirable to obtain a therapeutic benefit in a patient. A therapeutic benefit is not necessarily a cure for a particular disease or condition, but rather, preferably encompasses a result which can include alleviation of the disease or condition, elimination of the disease or condition, reduction of a symptom associated with the disease or condition, prevention or alleviation of a secondary disease or condition resulting from the occurrence of a primary disease or condition, and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or 5 causes. As such, to protect a patient from a disease includes both preventing disease occurrence (prophylactic treatment) and treating a patient that has a disease (therapeutic treatment) to reduce the symptoms of the disease. A beneficial effect can easily be assessed by one of ordinary skill in the art and/or by a trained clinician who is treating the patient. The term, “disease” refers to any deviation from the normal health of a mammal 10 and includes a state when disease symptoms are present, as well as conditions in which a deviation (e.g., infection, gene mutation, genetic defect, etc.) has occurred, but symptoms are not yet manifested.

### Kits

The present application concerns kits for use with the compounds described herein. In some embodiments, the invention provides a kit including an EGFR inhibitor in a dosage form, especially a dosage form for oral administration. In some embodiments, the kit further includes a ZEB1 and/or SIP1 inhibitor in a dosage form, especially a dosage form for oral administration. In specific embodiments, the EGFR inhibitor and ZEB1 and/or SIP1 inhibitor are in separate dosage forms. In some embodiments of the invention, the kit includes one or more doses of an EGFR inhibitor in tablets for oral administration. In other embodiments, however, the dose or doses an EGFR inhibitor may be present in a variety of dosage forms, such as capsules, caplets, gel caps, powders for suspension, etc. In some embodiments, the kit includes a liquid dosage form for the EGFR inhibitor. In some embodiments the EGFR inhibitor is in a dosage form suitable for intravenous delivery or dosage. In some embodiments of the invention, the kit includes one or more doses of an EGFR inhibitor in tablets for oral administration. In other embodiments, however, the dose or doses of an EGFR inhibitor may be present in a variety of dosage forms, such as capsules, caplets, gel caps, powders for suspension, etc.

In some embodiments, a kit according to the invention includes at least three dosage forms, one comprising an EGFR inhibitor, one comprising a ZEB1 and/or SIP1 inhibitor and the other comprising at least a third active pharmaceutical ingredient, other than the EGFR inhibitor and the ZEB1 and/or SIP1 inhibitor pharmaceutical ingredient. In some embodiments, the third active pharmaceutical ingredient is a second EGFR inhibitor. In other embodiments, the third active pharmaceutical ingredient is a second ZEB1 and/or SIP1 inhibitor. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. It is considered that the most convenient periods of time for which such kits are designed would be from 1 to 13 weeks, especially 1 week, 2 weeks, 1 month, 3 months, etc. In some specific embodiments, the each dose is physically separated into a compartment, in which each dose is segregated from the others.

In some embodiments, the kit according to the invention includes at least two dosage forms one comprising an EGFR inhibitor and one comprising an ZEB1 and/or SIP1 inhibitor. In some embodiments, the kit according to the invention includes at least two dosage forms where one comprises an EGFR inhibitor and the other comprises at least a second dosage form of an active pharmaceutical ingredient, other than the EGFR inhibitor. In some embodiments, the kit according to the invention includes at least two dosage forms comprising two different EGFR inhibitors. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. In some specific embodiments, the each dose is physically separated into a compartment, in which each dose is segregated from the others.

In some embodiments, the kit according to the invention includes at least one dosage form comprising at least one EGFR inhibitor. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. In some specific embodiments, the each dose is physically separated into a compartment, in which each dose is segregated from the others.

In particular embodiments, the kit may advantageously be a blister pack. Blister packs are known in the art, and generally include a clear side having compartments (blisters or bubbles), which separately hold the various doses, and a backing, such as a paper, foil, paper-foil or other backing, which is easily removed so that each dose may be separately extracted from the blister pack without disturbing the other doses. In some embodiments, the kit may be a blister pack in which each dose of the EGFR inhibitor, the ZEB1 and/or SIP1 inhibitor and, optionally, a third active pharmaceutical ingredient are segregated from the other doses in separate blisters or bubbles. In some such embodiments, the blister pack may have perforations, which allow each daily dose to be separated from the others by tearing it away from the rest of the blister pack. The separate dosage forms may be contained within separate blisters. Segregation of the active pharmaceutical ingredients into separate blisters can be advantageous in that it prevents separate dosage forms (e.g., tablet and capsule) from contacting and damaging one another during shipping and handling. Additionally, the separate dosage forms can be accessed and/or labeled for administration to the patient at different times.

In some embodiments, the kit may be a blister pack in which each separate dose the EGFR inhibitor, the ZEB1 and/or SIP1 inhibitor and, optionally, a third active pharmaceutical ingredient is segregated from the other doses in separate blisters or bubbles. In another embodiment, the kit includes two different EGFR inhibitors and, optionally, a third active pharmaceutical ingredient. In some such embodiments, the blister pack may have perforations, which allow each daily dose to be separated from the others by tearing it away from the rest of the blister pack. The separate dosage forms may be contained within separate blisters.

In some embodiments, one or more of the active pharmaceutical ingredients may be in the form of a liquid or a reconstitutable powder, which may be separately sealed (e.g., in a vial or ampoule) and then packaged along with a blister pack containing separate dosages of the EGFR inhibitor and the ZEB1 and/or SIP1 inhibitor. In some embodiments, the EGFR inhibitor is in the form of a liquid or reconstitutable powder that is separately sealed (e.g., in a vial or ampoule). In some embodiments, the EGFR inhibitor is in the form of a liquid or reconstitutable powder that is separately sealed (e.g., in a vial or ampoule) and then packaged along with a blister pack containing separate dosages of the ZEB1 and/or SIP1 inhibitor. These embodiments would be especially useful in a clinical setting where prescribed doses of the EGFR inhibitor, ZEB1 and/or SIP1 inhibitor and, optionally, a third active pharmaceutically active agent would be used on a dosing schedule in which the ZEB1 and/or SIP1 inhibitor is administered on certain days, the EGFR inhibitor is administered on the same or different days and the third active pharmaceutical ingredient is administered on the same or different days from either or both of the ZEB1 and/or SIP1 and/or EGFR inhibitors within a weekly, bi-weekly, 2× weekly or other dosing schedule. Such a combination of blister pack containing an EGFR inhibitor, a ZEB1 and/or SIP1 inhibitor and an optional third active pharmaceutical agent could also include instructions for administering each of the EGFR inhibitor, a ZEB1 and/or SIP1 inhibitor and the optional third active pharmaceutical agent on a dosing schedule adapted to provide the synergistic treating effect of the EGFR inhibitor and/or the third active pharmaceutical agent.

In other embodiments, the kit may be a container having separate compartments with separate lids adapted to be opened on a particular schedule. For example, a kit may comprise a box (or similar container) having seven compartments, each for a separate day of the week, and each compartment marked to indicate which day of the week it corresponds to. In some specific embodiments, each compartment is further subdivided to permit segregation of one active pharmaceutical ingredient from another. As stated above, such segregation is advantageous in that it prevents damage to the dosage forms and permits dosing at different times and labeling to that effect. Such a container could also include instructions for administering an EGFR inhibitor, a ZEB1 and/or SIP1 inhibitor and the optional third active pharmaceutical ingredient on a dosing schedule adapted to provide the synergistic treating effect of the EGFR inhibitor and/or the third active pharmaceutical ingredient.

In some embodiments, wherein the packaging material further comprises a container for housing the pharmaceutical composition, the kit comprises an EGFR inhibitor composition that is in a different physical location within the kit from a ZEB1 and/or SIP1 inhibitor composition. In further embodiments, the kit comprises a third pharmaceutical agent that is in a separate physical location from either the EGFR inhibitor composition or the ZEB1 and/or SIP1 inhibitor composition. In some embodiments, the different physical locations of ZEB1 and/or SIP1 inhibitor composition and the EGFR inhibitor composition comprise separately sealed individual compartments. In certain embodiments, the kit comprises an EGFR inhibitor composition that is in a first separately sealed individual compartment and a ZEB1 and/or SIP1 inhibitor composition that is in a second separately sealed individual compartment. In embodiments wherein the EGFR inhibitor composition and ZEB1 and/or SIP1 inhibitor composition compartments are separate, the different locations are used, e.g., to distinguish between the ZEB1 and/or SIP1 inhibitor composition and EGFR inhibitor compositions. In further embodiments, a third pharmaceutical composition is in a third physical location within the kit.

The container means of the kits will generally include at least one vial, test tube, flask, bottle, syringe and/or other container means, into which the at least one polypeptide can be placed, and/or preferably, suitably aliquoted. The kits can include a means for containing at least one detectable moiety, reporter molecule, and/or any other reagent containers in close confinement for commercial sale. Such containers may include injection and/or blow-molded plastic containers in which the desired vials are stored. Kits can also include printed material for use of the materials in the kit.

Packages and kits can additionally include a buffering agent, a preservative and/or a stabilizing agent in a pharmaceutical formulation. Each component of the kit can be enclosed within an individual container and all of the various containers can be within a single package. Invention kits can be designed for cold storage or room temperature storage.

Additionally, the preparations can contain stabilizers (such as bovine serum albumin (BSA)) to increase the shelf-life of the kits. Where the compositions are lyophilized, the kit can contain further preparations of solutions to reconstitute the lyophilized preparations. Acceptable reconstitution solutions are well known in the art and include, for example, pharmaceutically acceptable phosphate buffered saline (PBS).

Additionally, the packages or kits provided herein can further include any of the other moieties provided herein such as, for example, one or more reporter molecules and/or one or more detectable moieties/agents.

Packages and kits can further include one or more components for an assay, such as, for example, an ELISA assay, cytotoxicity assay, etc. Samples to be tested in this application include, for example, blood, plasma, and tissue sections and secretions, urine, lymph, and products thereof. Packages and kits can further include one or more components for collection of a sample (e.g., a syringe, a cup, a swab, etc.).

Packages and kits can further include a label specifying, for example, a product description, mode of administration and/or indication of treatment. Packages provided herein can include any of the compositions as described herein for treatment of any of the indications described herein.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions. Kits, therefore, can additionally include labels or instructions for using the kit components in any method of the invention. A kit can include a compound in a pack, or dispenser together with instructions for administering the compound in a method described herein.

The kits may also include instructions teaching the use of the kit according to the various methods and approaches described herein. Such kits optionally include information, such as scientific literature references, package insert materials, clinical trial results, and/or summaries of these and the like, which indicate or establish the activities and/or advantages of the composition, and/or which describe dosing, administration, side effects, drug interactions, disease state for which the composition is to be administered, or other information useful to the health care provider. Such information may be based on the results of various studies, for example, studies using experimental animals involving in vivo models and studies based on human clinical trials. In various embodiments, the kits described herein can be provided, marketed and/or promoted to health providers, including physicians, nurses, pharmacists, formulary officials, and the like. Kits may, in some embodiments, be marketed directly to the consumer. In certain embodiments, the packaging material further comprises a container for housing the composition and optionally a label affixed to the container. The kit optionally comprises additional components, such as but not limited to syringes for administration of the composition.

Instructions can include instructions for practicing any of the methods described herein including treatment methods. Instructions can additionally include indications of a satisfactory clinical endpoint or any adverse symptoms that may occur, or additional information required by regulatory agencies such as the Food and Drug Administration for use on a human subject.

The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM, IC tip and hybrids of these such as magnetic/optical storage media.

The invention thus provides kits which utilize the diagnostic methods and assays described herein. In some embodiments, a kit according to the invention comprises reagents for the detection of a gene or genes whose expression levels have been correlated with sensitivity or resistance to an EGFR inhibitor in a sample of cancer cells from a patient. In some embodiments, the gene or genes are selected from E-cadherin, ErbB3 (Hera), RAB25, integrin beta 6, cadherein-2 (N-cadherin), fibroblast growth factor binding protein 1, distal-less homeo box 1, vimentin, ZEB1, and SIP1. In some embodiments, the kit comprises E-cadherin. In some embodiments, the kit comprises ErbB3. In some embodiments, the kit comprises RAB25. In some embodiments, the kit comprises integrin beta 6. In some embodiments, the kit comprises cadherin-2. In some embodiments, the kit comprises fibroblast growth factor binding protein 1. In some embodiments, the kit comprises distal-less homeo box 1. In some embodiments, the kit comprises vimentin. In some embodiments, the kit comprises ZEB1. In some embodiments, the kit comprises SIP1. In some embodiments, the kit comprises at least two of E-cadherin, ErbB3 (Her3), RAB25, integrin beta 6, cadherein-2 (N-cadherin), fibroblast growth factor binding protein 1, distal-less homeo box 1, vimentin, ZEB1, and SIP1. In some embodiments, the kit comprises at least two genes that have been correlated with sensitivity to an EGFRi. In some embodiments, the kit comprises at least two genes that have been correlated with resistance to an EGFRi. In some embodiments, the kit comprises at least one gene that has been correlated with sensitivity to an EGFRi and on gene that has been correlated with resistance to an EGFRi.

In still further embodiments, a kit according to the invention comprises reagents for the detection of E-cadherin, ErbB3 (Her3), RAB25, integrin beta 6, cadherein-2 (N-cadherin), fibroblast growth factor binding protein 1, distal-less homeo box 1, vimentin, ZEB1, and SIP1 expression levels in a sample of tumor cells from a patient to be treated; and a dose or doses an EGFR inhibitor in a variety of dosage forms, such as capsules, caplets, gel caps, powders for suspension, etc. It is further contemplated within the invention that kit comprising reagents for the detection of E-cadherin, ErbB3 (Her3), RAB25, integrin beta 6, cadherein-2 (N-cadherin), fibroblast growth factor binding protein 1, distal-less homeo box 1, vimentin, ZEB1, and SIP1 expression levels in a sample of tumor cells from a patient to be treated will further comprise any of the aforementioned embodiments of the kits for co-administration of the EGFR inhibitor. In some embodiments, the kit comprises E-cadherin. In some embodiments, the kit comprises ErbB3. In some embodiments, the kit comprises RAB25. In some embodiments, the kit comprises integrin beta 6. In some embodiments, the kit comprises cadherin-2. In some embodiments, the kit comprises fibroblast growth factor binding protein 1. In some embodiments, the kit comprises distal-less homeo box 1. In some embodiments, the kit comprises vimentin. In some embodiments, the kit comprises ZEB1. In some embodiments, the kit comprises SIP1. In some embodiments, the kit comprises at least two of E-cadherin, ErbB3 (Her3), RAB25, integrin beta 6, cadherein-2 (N-cadherin), fibroblast growth factor binding protein 1, distal-less homeo box 1, vimentin, ZEB1, and SIP1. In some embodiments, the kit comprises at least two genes that have been correlated with sensitivity to an EGFRi. In some embodiments, the kit comprises at least two genes that have been correlated with resistance to an EGFRi. In some embodiments, the kit comprises at least one gene that has been correlated with sensitivity to an EGFRi and on gene that has been correlated with resistance to an EGFRi.

Kits can, in some aspects, contain reagents and materials to conduct any of the assays described herein.

It is understood that the kits provided by the invention and described herein can include any of the embodiments for the detection of genes as described herein. It is also understood that the kits can include any of the embodiments for the correlation of resistance or sensitivity to any of the EGFR inhibitors as described herein. Finally, it is understood that the kits can also include any of the embodiments for the detection of genes and correlation of sensitivity or resistance to EGFR inhibitors in any of the cancers as described herein.

In some embodiments, the gene detected is E-cadherin. In some embodiments, the gene detected is RAB25. In some embodiments, the gene detected is integrin beta 6. In some embodiments, the gene detected is vimentin. In some embodiments, the gene detected is ZEB1. In some embodiments, the gene detected is SIP1. In still further embodiments, the gene or genes detected are E-cadherein, ZEB1 and SIP1. In further embodiments, the gene or genes detected are E-cadherin, RAB25, and integrin beta 6. In additional embodiments, the genes detected are ZEB1 and vimentin. Additional combinations of the genes described herein are useful in additional embodiments of the kits of the invention and are contemplated herein.

Various aspects of the invention are described in the following examples; however, the following examples are provided for the purpose of illustration and are not intended to limit the scope of the present invention.

### EXAMPLES

### Example 1

The following example describes the identification of a biomarker panel that discriminates EGFR inhibitor-sensitive cell lines from EGFR inhibitor-resistant cell lines.

Methods: EGFR inhibitor sensitivity is determined in 18 NSCLC cell lines using MTT assays. Cell lines are classified as EGFR inhibitor sensitive (IC₅₀<1 μM), resistant (IC₅₀>10 μM) or intermediate sensitivity (10 μM<IC₅₀>1). Oligonucleotide gene arrays (Affymetrix® Human Genome U133 set, 39,000 genes) are done on 10 cell lines. Three distinct filtration and normalization algorithms to process the expression data are used, and a list of genes is generated that is both statistically significant (unadjusted p=0.001 cutoff) and corrected for false positive occurrence. This approach is used in combination with 5 distinct machine learning algorithms used to build a test set for predictor genes that are successful for 100% of the test cases. The best discriminators (>3 fold difference in expression between sensitive and resistant cell lines) are selected for Real-time RT-PCR.

Results: A list of genes is generated initially from the Affymetrix array analysis. By using the mathematical algorithm, 10-30 different candidate genes are selected for RT-PCR.

Conclusion: Based on NSCLC cell line studies it is possible to identify genes which strongly discriminate EGFR inhibitor sensitive cell lines (Table 1-Sensitive) from the EGFR inhibitor resistant cell lines (Table 1-Resistant). The genes are ranked in Table 1. This entire biomarker panel is of significant value for selecting NSCLC patients for EGFR inhibitor treatment.

TABLE 1

| | | | | |
|---|---|---|---|---|
| Probe | parametric | Gene | Sequence | |
| set | p-value | symbol | Identifier | Description |

Sensitive

| | | | | |
|---|---|---|---|---|
| 202286 s | 0.00000005 | TACSTD2 | SEQ ID | tumor-associated calcium signal transducer 2 |
| at | | | NO: 12 | |
| 202489_s_at | 0.00000005 | FXYD3 | SEQ ID | FXYD domain containing ion transport regulator 3 |
| | | | NO: 16 | |
| 213285 | 0.00000005 | TMEM30B | SEQ ID | transmembrane protein 30B |
| at | | | NO: 73 | |
| 218186 | 0.00000005 | RAB25 | SEQ ID | RAB25, member RAS oncogene family |
| at | | | NO: 83 | |
| 235515 | 0.00000005 | FLJ36445 | SEQ ID | hypothetical protein FLJ36445 |
| at | | | NO: 168 | |
| 235988 | 0.00000005 | GPR110 | SEQ ID | G protein-coupled receptor 110 |
| at | | | NO: 170 | |
| 238689 | 0.00000005 | GPR110 | SEQ ID | G protein-coupled receptor 110 |
| at | | | NO: 177 | |
| 232165 | 0.00000010 | EPPK1 | SEQ ID | epiplakin 1 |
| at | | | NO: 164 | |
| 240633 | 0.00000010 | FLJ33718 | SEQ ID | hypothetical protein FLJ33718 |
| at | | | NO: 182 | |
| 229599_at | 0.00000020 | | SEQ ID | Clone IMAGE: 5166045, Mrna |
| | | | NO: 154 | |
| 203397_s_at | 0.00000030 | GALNT3 | SEQ ID | UDP-N-acetyl-alpha-D-galactosamine:polypeptide |
| | | | NO: 28 | N-acetylgalactosaminyltransferase 3 (GalNAc-T3) |
| 232164 s | 0.00000030 | EPPK1 | SEQ ID | epiplakin 1 |
| at | | | NO: 163 | |
| 227134 | 0.00000160 | SYTL1 | SEQ ID | synaptotagmin-like 1 |
| at | | | NO: 143 | |
| 236489 | 0.00000170 | | SEQ ID | |
| at | | | NO: 171 | |
| 235651 | 0.00000480 | | SEQ ID | |
| at | | | NO: 169 | |
| 238439 | 0.00000700 | ANKRD22 | SEQ ID | ankyrin repeat domain 22 |
| at | | | NO: 173 | |
| 219388 | 0.00000730 | TFCP2L3 | SEQ ID | transcription factor CP2-like 3 |
| at | | | NO: 91 | |
| 227985 | 0.00000820 | | SEQ ID | |
| at | | | NO: 146 | |
| 227450 | 0.00000890 | FLJ32115 | SEQ ID | hypothetical protein FLJ32115 |
| at | | | NO: 144 | |
| 203256 | 0.00000980 | CDH3 | SEQ ID | cadherin 3, type 1, P-cadherin (placental) |
| at | | | NO: 23 | |
| 220318 | 0.00000980 | EPN3 | SEQ ID | epsin 3 |
| at | | | NO: 100 | |
| 202525 | 0.00001030 | PRSS8 | SEQ ID | protease, serine, 8 (prostasin) |
| at | | | NO: 17 | |
| 227803_at | 0.00001080 | ENPP5 | SEQ ID | ectonudeotide pyrophosphatase/phosphodiesterase 5 |
| | | | NO: 145 | (putative function) |
| 206884 s | 0.00001200 | SCEL | SEQ ID | Sciellin |
| at | | | NO: 49 | |
| 223895 s | 0.00001290 | EPN3 | SEQ ID | epsin 3 |
| at | | | NO: 119 | |
| 238493 | 0.00001650 | ZNF506 | SEQ ID | zinc finger protein 506 |
| at | | | NO. 174 | |
| 201428 | 0.00002330 | CLDN4 | SEQ ID | claudin 4 |
| at | | | NO: 5 | |
| 216641 s | 0.00003760 | LAD1 | SEQ ID | ladinin 1 |
| at | | | NO: 78 | |
| 231929_at | 0.00003910 | | SEQ ID | MRNA; cDNA DKFZp586O0724 (from clone |
| | | | NO: 159 | DKFZp586O0724) |
| 238778_at | 0.00004080 | MPP7 | SEQ ID | membrane protein, palmitoylated 7 (MAGUK p55 |
| | | | NO: 178 | subfamily member 7) |
| 203287 | 0.00004920 | LAD1 | SEQ ID | ladinin 1 |
| at | | | NO: 24 | |
| 209114 | 0.00005560 | TSPAN-1 | SEQ ID | tetraspan 1 |
| at | | | NO: 57 | |
| 230076 | 0.00005660 | | SEQ ID | |
| at | | | NO: 155 | |
| 218677 | 0.00005710 | S100A14 | SEQ ID | S100 calcium binding protein A14 |
| at | | | NO: 85 | |
| 236616 | 0.00005810 | | SEQ ID | CDNA FLJ41623 fis, clone CTONG3009227 |
| at | | | NO: 172 | |
| 205014 | 0.00006280 | FGFBP1 | SEQ ID | fibroblast growth factor binding protein 1 |
| at | | | NO: 40 | |
| 90265 at | 0.00007110 | CENTA1 | SEQ ID | centaurin, alpha 1 |
| | | | NO: 193 | |
| 226403 | 0.00007930 | TMC4 | SEQ ID | transmembrane channel-like 4 |
| at | | | NO: 136 | |
| 232056 | 0.00008450 | SCEL | SEQ ID | Scieliin |
| at | | | NO: 160 | |
| 207655 s | 0.00008700 | BLNK | SEQ ID | B-cell linker |
| at | | | NO: 51 | |
| 204160_s_at | 0.00009570 | ENPP4 | SEQ ID | Ectonucleotide pyrophosphatase/phosphodiesterase 4 |
| | | | NO: 36 | (putative function) |
| 202454_s_at | 0.00009860 | ERBB3 | SEQ ID | v-erb-b2 erythroblastic leukemia viral oncogene |
| | | | NO: 15 | homolog 3 (avian) |
| 232151_at | 0.00010020 | | SEQ ID | MRNA full length insert cDNA clone |
| | | | NO: 162 | EUROIMAGE 2344436 |
| 205073_at | 0.00010350 | CYP2J2 | SEQ ID | cytochrome P450, family 2, subfamily J, polypeptide 2 |
| | | | NO: 41 | |
| 225658 | 0.00011660 | LOC339745 | SEQ ID | hypothetical protein LOC339745 |
| at | | | NO: 127 | |
| 219150 s | 0.00012240 | CENTA1 | SEQ ID | centaurin, alpha 1 |
| at | | | NO: 90 | |
| 222857_s_at | 0.00012430 | KCNMB4 | SEQ ID | potassium large conductance calcium-activated |
| | | | NO: 113 | channel, subfamily M, beta member 4 |
| 55662 at | 0.00013490 | C10orf76 | SEQ ID | chromosome 10 open reading frame 76 |
| | | | NO: 191 | |
| 204161_s_at | 0.00013900 | ENPP4 | SEQ ID | Ectonucleotide pyrophosphatase/phosphodiesterase 4 |
| | | | NO: 37 | (putative function) |
| 205455_at | 0.00014640 | MST1R | SEQ ID | macrophage stimulating 1 receptor (c-met-related |
| | | | NO: 42 | tyrosine kinase) |
| 235247 | 0.00019200 | | SEQ ID | |
| at | | | NO: 167 | |
| 205617 | 0.00019960 | PRRG2 | SEQ ID | proline rich Gla (G-carboxyglutamic acid) 2 |
| at | | | NO: 44 | |
| 225822 | 0.00020110 | MGC17299 | SEQ ID | hypothetical protein MGC17299 |
| at | | | NO: 129 | |
| 218779 x | 0.00021870 | EPS8L1 | SEQ ID | EPS8-like 1 |
| at | | | NO: 86 | |
| 218792 s | 0.00023140 | BSPRY | SEQ ID | B-box and SPRY domain containing |
| at | | | NO: 87 | |
| 203236_s_at | 0.00025890 | LGALS9 | SEQ ID | 11 lectin, galactoside-binding, soluble, 9 (galectin 9) |
| | | | NO: 22 | |
| 229223 | 0.00026990 | | SEQ ID | |
| at | | | NO: 152 | |
| 226187_at | 0.00027300 | CDS1 | SEQ ID | CDP-diacylglycerol synthase (phosphatidate |
| | | | NO: 132 | cytidylyltransferase) 1 |
| 239671 | 0.00028050 | | SEQ ID | CDNA FLJ31085 fis, clone IMR321000037 |
| at | | | NO: 181 | |
| 222746 s | 0.00028540 | BSPRY | SEQ ID | B-box and SPRY domain containing |
| at | | | NO: 111 | |
| 219858 s | 0.00029160 | FLJ20160 | SEQ ID | FLJ20160 protein |
| at | | | NO: 96 | |
| 210749 x | 0.00029280 | DDR1 | SEQ ID | discoidin domain receptor family, member 1 |
| at | | | NO: 59 | |
| 211778 s | 0.00029620 | ZNF339 | SEQ ID | zinc finger protein 339 /// zinc finger protein 339 |
| at | | | NO: 61 | |
| 230323 s | 0.00033140 | LOC120224 | SEQ ID | hypothetical protein BC016153 |
| at | | | NO: 157 | |
| 221665 s | 0.00033480 | EPS8L1 | SEQ ID | EPS8-like 1 |
| at | | | NO: 105 | |
| 1007 s at | 0.00033840 | DDR1 | SEQ ID | discoidin domain receptor family, member 1 |
| | | | NO: 1 | |
| 218960 | 0.00034100 | TMPRSS4 | SEQ ID | transmembrane protease, serine 4 |
| at | | | NO: 89 | |
| 226213 | 0.00036180 | ERBB3 | SEQ ID | v-erb-b2 erythroblastic leukemia viral oncogene |
| at | | | NO: 133 | homolog 3 (avian) |
| 202597 | 0.00037880 | IRF6 | SEQ ID | interferon regulatory factor 6 |
| at | | | NO: 18 | |
| 228865 | 0.00037970 | SARG | SEQ ID | specifically androgen-regulated protein |
| at | | | NO: 149 | |
| 205709_s_at | 0.00038120 | CDS1 | SEQ ID | CDP-diacylglycerol synthase (phosphatidate |
| | | | NO: 45 | cytidylyltransferase) 1 |
| 224946 s | 0.00039420 | MGC12981 | SEQ ID | hypothetical protein MGC12981 |
| at | | | NO: 123 | |
| 204856_at | 0.00039710 | B3GNT3 | SEQ ID | UDP-GlcNAc:betaGal beta-1,3-N- |
| | | | NO: 39 | acetylglucosaminyltransferase 3 |
| 203317 | 0.00039900 | PSD4 | SEQ ID | pleckstrin and Sec7 domain containing 4 |
| at | | | NO: 25 | |
| 221958 s | 0.00040170 | FLJ23091 | SEQ ID | putative NFkB activating protein 373 |
| at | | | NO: 106 | |
| 201130 s | 0.00040570 | CDH1 | SEQ ID | cadherin 1, type 1, E-cadherin (epithelial) |
| at | | | NO: 3 | |
| 205847 | 0.00042390 | PRSS22 | SEQ ID | protease, serine, 22 |
| at | | | NO: 47 | |
| 226535 | 0.00044520 | ITGB6 | SEQ ID | integrin, beta 6 |
| at | | | NO: 137 | |
| 65517 at | 0.00045130 | AP1M2 | SEQ ID | adaptor-related protein complex 1, mu 2 subunit |
| | | | NO: 192 | |
| 91826 at | 0.00045430 | EPS8L1 | SEQ ID | EPS8-like 1 |
| | | | NO: 194 | |
| 238673 | 0.00045640 | | SEQ ID | |
| at | | | NO: 176 | |
| 221610 s | 0.00046860 | STAP2 | SEQ ID | signal-transducing adaptor protein-2 |
| at | | | NO: 104 | |
| 203779 s | 0.00047400 | EVA1 | SEQ ID | epithelial V-like antigen 1 |
| at | | | NO: 33 | |
| 222830 | 0.00047770 | TFCP2L2 | SEQ ID | transcription factor CP2-like 2 |
| at | | | NO. H2 | |
| 203780 | 0.00047790 | EVA1 | SEQ ID | epithelial V-like antigen 1 |
| at | | | NO: 34 | |
| 223233 s | 0.00048700 | CGN | SEQ ID | cingulin |
| at | | | NO: 117 | |
| 219412 | 0.00049410 | RAB38 | SEQ ID | RAB38, member RAS oncogene family |
| at | | | NO: 92 | |
| 219936 s | 0.00049770 | GPR87 | SEQ ID | G protein-coupled receptor 87 |
| at | | | NO: 97 | |
| 226226 | 0.00049820 | LOC120224 | SEQ ID | hypothetical protein BC016153 |
| at | | | NO: 134 | |
| 225911 | 0.00050990 | LOC255743 | SEQ ID | hypothetical protein LOC255743 |
| at | | | NO: 130 | |
| 226584 s | 0.00053900 | C20orf55 | SEQ ID | chromosome 20 open reading frame 55 |
| at | | | NO: 138 | |
| 208779 x | 0.00054830 | DDR1 | SEQ ID | discoidin domain receptor family, member 1 |
| at | | | NO: 55 | |
| 208084 | 0.00055660 | ITGB6 | SEQ ID | integrin, beta 6 |
| at | | | NO: 52 | |
| 226678 | 0.00058120 | UNC13D | SEQ ID | unc-13 homolog D (C. elegans) |
| at | | | NO: 139 | |
| 216949_s_at | 0.00058240 | PKD1 | SEQ ID | polycystic kidney disease 1 (autosomal dominant) |
| | | | NO: 80 | |
| 212338 | 0.00058710 | MYO1D | SEQ ID | myosin ID |
| at | | | NO: 67 | |
| 241455 | 0.00059440 | | SEQ ID | |
| at | | | NO: 183 | |
| 206043 s | 0.00063910 | KIAA0703 | SEQ ID | KIAA0703 gene product |
| at | | | NO: 48 | |
| 226706 | 0.00063930 | FLJ23867 | SEQ ID | hypothetical protein FLJ23867 |
| at | | | NO: 140 | |
| 210255 | 0.00064190 | RAD51L1 | SEQ ID | RAD51-like 1 (S. cerevisiae) |
| at | | | NO: 58 | |
| 203407 | 0.00068500 | PPL | SEQ ID | periplakin |
| at | | | NO: 29 | |
| 222859_s_at | 0.00072460 | DAPP1 | SEQ ID | dual adaptor of phosphotyrosine and 3- |
| | | | NO: 114 | phosphoinositides |
| 219856 | 0.00075780 | SARG | SEQ ID | specifically androgen-regulated protein |
| at | | | NO: 95 | |
| 38766 at | 0.00075940 | SRCAP | SEQ ID | Snf2-related CBP activator protein |
| | | | NO: 189 | |
| 239196 | 0.00076210 | ANKRD22 | SEQ ID | ankyrin repeat domain 22 |
| at | | | NO: 180 | |
| 32069 at | 0.00077000 | N4BP1 | SEQ ID | Nedd4 binding protein 1 |
| | | | NO: 187 | |
| 205780 | 0.00083050 | | SEQ ID | |
| at | | | NO: 46 | |
| 238513_at | 0.00083510 | TMG4 | SEQ ID | transmembrane gamma-carboxyglutamic acid |
| at | | | NO: 175 | protein 4 |
| 229030 | 0.00084650 | | SEQ ID | |
| at | | | NO: 151 | |
| 226400 | 0.00088590 | | SEQ ID | |
| at | | | NO: 135 | |
| 228441 s | 0.00093570 | | SEQ ID | |
| at | | | NO: 147 | |
| 243302 | 0.00096750 | | SEQ ID | |
| at | | | NO: 186 | |

Resistant

| | | | | |
|---|---|---|---|---|
| 219525 | 0.00000020 | FLJ10847 | SEQ ID | hypothetical protein FLJ10847 |
| at | | | NO: 93 | |
| 212813 | 0.00000060 | JAM3 | SEQ ID | junctional adhesion molecule 3 |
| at | | | NO: 71 | |
| 224913 s | 0.00001960 | TIMM50 | SEQ ID | translocase of inner mitochondrial membrane 50 |
| at | | | NO: 122 | homolog (yeast) |
| 212764_at | 0.00003930 | TCF8 | SEQ ID | transcription factor 8 (represses interleukin 2 |
| | | | NO: 70 | expression) |
| 202641 | 0.00004360 | ARL3 | SEQ ID | ADP-ribosylation factor-like 3 |
| at | | | NO: 19 | |
| 212233 | 0.00004550 | MAP1B | SEQ ID | microtubule-associated protein 1B |
| at | | | NO: 66 | |
| 224232 s | 0.00004560 | PX19 | SEQ ID | px19-like protein |
| at | | | NO: 120 | |
| 226905 | 0.00004590 | MGC45871 | SEQ ID | hypothetical protein MGC45871 |
| at | | | NO: 142 | |
| 218553_s_at | 0.00004620 | KCTD15 | SEQ ID | potassium channel tetramerisation domain |
| | | | NO: 84 | containing 15 |
| 215218 s | 0.00004830 | C19orf14 | SEQ ID | chromosome 19 open reading frame 14 |
| at | | | NO: 77 | |
| 200720_s_at | 0.00006360 | ACTR1A | SEQ ID | ARP1 actin-related protein 1 homolog A, centractin |
| | | | NO: 2 | alpha (yeast) |
| 224326 s | 0.00006750 | RNF134 | SEQ ID | ring finger protein 134 /// ring finger protein 134 |
| at | | | NO: 121 | |
| 242138 | 0.00006800 | DLX1 | SEQ ID | distal-less homeo box 1 |
| at | | | NO: 184 | |
| 222360 | 0.00007190 | CGI-30 | SEQ ID | CGI-30 protein |
| at | | | NO: 108 | |
| 208393 s | 0.00007530 | RAD50 | SEQ ID | RAD50 homolog (S. cerevisiae) |
| at | | | NO: 53 | |
| 228683 s | 0.00009450 | KCTD15 | SEQ ID | potassium channel tetramerisation domain |
| at | | | NO: 148 | containing 15 |
| 228882 | 0.00012370 | TUB | SEQ ID | tubby homolog (mouse) |
| at | | | NO: 150 | |
| 55662 at | 0.00013490 | C10orf76 | SEQ ID | chromosome 10 open reading frame 76 |
| | | | NO: 191 | |
| 221432_s_at | 0.00014780 | SLC25A28 | SEQ ID | solute carrier family 25, member 28 /// solute carrier |
| | | | NO: 102 | family 25, member 28 |
| 203082 | 0.00015630 | BMS1L | SEQ ID | BMS1-like, ribosome assembly protein (yeast) |
| at | | | NO: 20 | |
| 223192 | 0.00015890 | SLC25A28 | SEQ ID | solute carrier family 25, member 28 |
| at | | | NO: 116 | |
| 226084 | 0.00017240 | MAP1B | SEQ ID | microtubule-associated protein 1B |
| at | | | NO: 131 | |
| 229587 | 0.00017530 | UBA2 | SEQ ID | SUMO-1 activating enzyme subunit 2 |
| at | | | NO. 153 | |
| 211071_s_at | 0.00018080 | AF1Q | SEQ ID | ALL1-fused gene from chromosome 1q /// ALL1- |
| | | | NO: 60 | fused gene from chromosome 1q |
| 214448_x_at | 0.00018290 | NFKBIB | SEQ ID | nuclear factor of kappa light polypeptide gene |
| | | | NO: 74 | enhancer in B-cells inhibitor, beta |
| 225413 | 0.00018660 | USMG5 | SEQ ID | upregulated during skeletal muscle growth 5 |
| at | | | NO: 125 | |
| 235036 | 0.00018930 | MGC46719 | SEQ ID | hypothetical protein MGC46719 |
| at | | | NO: 165 | |
| 203441 s | 0.00019180 | CDH2 | SEQ ID | cadherin 2, type 1, N-cadherin (neuronal) |
| at | | | NO: 31 | |
| 225096 | 0.00019610 | HSA272196 | SEQ ID | hypothetical protein, clone 2746033 |
| at | | | NO: 124 | |
| 239077 | 0.00020310 | GALNACT-2 | SEQ ID | chondroitin sulfate GalNAcT-2 |
| at | | | NO: 179 | |
| 50314 i | 0.00022630 | C20orf27 | SEQ ID | chromosome 20 open reading frame 27 |
| at | | | NO: 190 | |
| 222664_at | 0.00024210 | KCTD15 | SEQ ID | potassium channel tetramerisation domain |
| | | | NO: 109 | containing 15 |
| 201869 s | 0.00024250 | TBL1X | SEQ ID | transducin (beta)-like 1X-linked |
| at | | | NO: 9 | |
| 219855_at | 0.00024820 | NUDT11 | SEQ ID | nudix (nucleoside diphosphate linked moiety X)- |
| | | | NO: 94 | type motif |
| 202167 s | 0.00026530 | MMS19L | SEQ ID | MMS19-like (MET18 homolog, S. cerevisiae) |
| at | | | NO: 10 | |
| 201157 s | 0.00027160 | NMT1 | SEQ ID | N-myristoyltransferase 1 |
| at | | | NO: 4 | |
| 226876 | 0.00030570 | MGC45871 | SEQ ID | hypothetical protein MGC45871 |
| at | | | NO: 141 | |
| 218891 | 0.00034090 | C10orf76 | SEQ ID | chromosome 10 open reading frame 76 |
| at | | | NO: 88 | |
| 222668_at | 0.00034910 | KCTD15 | SEQ ID | potassium channel tetramerisation domain |
| | | | NO: 110 | containing 15 |
| 217496 s | 0.00036040 | IDE | SEQ ID | insulin-degrading enzyme |
| at | | | NO: 81 | |
| 235202 x | 0.00036460 | [KIP | SEQ ID | IKK interacting protein |
| at | | | NO: 166 | |
| 212736 | 0.00036600 | BC008967 | SEQ ID | hypothetical gene BC008967 |
| at | | | NO: 69 | |
| 203327 | 0.00036980 | IDE | SEQ ID | insulin-degrading enzyme |
| at | | | NO: 26 | |
| 205458_at | 0.00042200 | MC1R | SEQ ID | melanocortin 1 receptor (alpha melanocyte |
| | | | NO: 43 | stimulating hormone receptor) |
| 202340_x_at | 0.00043030 | NR4A1 | SEQ ID | nuclear receptor subfamily 4, group A, member 1 |
| | | | NO: 14 | |
| 215146 s | 0.00043080 | KIAA1043 | SEQ ID | KIAA1043 protein |
| at | | | NO: 76 | |
| 223032 x | 0.00043320 | PX19 | SEQ ID | px19-like protein |
| at | | | NO: 115 | |
| 230312 | 0.00047560 | | SEQ ID | |
| at | | | NO: 156 | |
| 211855_s_at | 0.00047620 | SLC25A14 | SEQ ID | solute carrier family 25 (mitochondrial carrier, |
| | | | NO: 62 | brain), member 14 |
| 222280 | 0.00050070 | | SEQ ID | CDNA clone IMAGE: 6602785, partial cds |
| at | | | NO: 107 | |
| 223295 s | 0.00053580 | LUC7L | SEQ ID | LUC7-like (S. cerevisiae) |
| at | | | NO: 118 | |
| 212120 | 0.00053760 | RHOQ | SEQ ID | ras homolog gene family, member Q |
| at | | | NO: 65 | |
| 202328_s_at | 0.00054270 | PKD1 | SEQ ID | polycystic kidney disease 1 (autosomal dominant) |
| | | | NO: 13 | |
| 203783 x | 0.00055660 | POLRMT | SEQ ID | polymerase (RNA) mitochondria! (DNA directed) |
| at | | | NO: 35 | |
| 213262 | 0.00056350 | SACS | SEQ ID | spastic ataxia of Charlevoix-Saguenay (sacsin) |
| at | | | NO: 72 | |
| 225793 | 0.00058010 | MGC46719 | SEQ ID | hypothetical protein MGC46719 |
| at | | | NO: 128 | |
| 216949_s_at | 0.00058240 | PKD1 | SEQ ID | polycystic kidney disease 1 (autosomal dominant) |
| | | | NO: 80 | |
| 214577 | 0.00062040 | MAP1B | SEQ ID | microtubule-associated protein 1B |
| at | | | NO: 75 | |
| 220178 | 0.00062110 | C19orf28 | SEQ ID | chromosome 19 open reading frame 28 |
| at | | | NO: 99 | |
| 201868 s | 0.00062220 | TBL1X | SEQ ID | transducin (beta)-like 1X-linked |
| at | | | NO: 8 | |
| 201679 | 0.00063150 | ARS2 | SEQ ID | arsenate resistance protein ARS2 |
| at | | | NO: 6 | |
| 208968 s | 0.00066500 | CIAPIN1 | SEQ ID | cytokine induced apoptosis inhibitor 1 |
| at | | | NO: 56 | |
| 207627 s | 0.00068160 | TFCP2 | SEQ ID | transcription factor CP2 |
| at | | | NO: 50 | |
| 217791 s | 0.00069580 | ALDH18A1 | SEQ ID | aldehyde dehydrogenase 18 family, member A1 |
| at | | | NO: 82 | |
| 225582 | 0.00069740 | KIAA1754 | SEQ ID | KIAA1754 |
| at | | | NO: 126 | |
| 231721 | 0.00070410 | JAM3 | SEQ ID | junctional adhesion molecule 3 |
| at | | | NO: 158 | |
| 208595 s | 0.00074160 | MBD1 | SEQ ID | methyl-CpG binding domain protein 1 |
| at | | | NO: 54 | |
| 212015 | 0.00075720 | PTBP1 | SEQ ID | polypyrimidine tract binding protein 1 |
| X at | | | NO: 63 | |
| P204744 | 0.00076150 | IARS | SEQ ID | isoleucine-tRNA synthetase |
| s at | | | NO: 38 | |
| 203718 | 0.00076760 | NTE | SEQ ID | neuropathy target esterase |
| at | | | NO: 32 | |
| 232149_s_at | 0.00076810 | NSMAF | SEQ ID | neutral sphingomyelinase (N-SMase) activation |
| | | | NO: 161 | associated factor |
| 202264_s_at | 0.00076920 | TOMM40 | SEQ ID | translocase of outer mitochondrial membrane 40 |
| | | | NO: 11 | homolog (yeast) |
| 32069 at | 0.00077000 | N4BP1 | SEQ ID | Nedd4 binding protein 1 |
| | | | NO: 187 | |
| 216862 s | 0.00078160 | MTCP1 | SEQ ID | mature T-cell proliferation 1 |
| at | | | NO: 79 | |
| 220370 s | 0.00079540 | USP36 | SEQ ID | ubiquitin specific protease 36 |
| at | | | NO: 101 | |
| 242191 | 0.00080180 | | SEQ ID | LOC400781 |
| at | | | NO: 185 | |
| 203109_at | 0.00081840 | UBE2M | SEQ ID | ubiquitin-conjugating enzyme E2M (UBC12 |
| | | | NO: 21 | homolog, yeast) |
| 203440 | 0.00\083250 | CDH2 | SEQ ID | cadherin 2, type 1, N-cadherin (neuronal) |
| at | | | NO: 30 | |
| 221550_at | 0.00083680 | COX15 | SEQ ID | COX15 homolog, cytochrome c oxidase assembly |
| | | | NO: 103 | protein (yeast) |
| 37966 at | 0.00090730 | PARVB | SEQ ID | parvin, beta |
| | | | NO: 188 | |
| 212424 | 0.00092430 | PDCD11 | SEQ ID | programmed cell death 11 |
| at | | | NO: 68 | |
| 228441 s | 0.00093570 | | SEQ ID | |
| at | | | NO: 147 | |
| 203328 x | 0.00095810 | IDE | SEQ ID | insulin-degrading enzyme |
| at | | | NO: 27 | |
| 201680 x | 0.00095980 | ARS2 | SEQ ID | arsenate resistance protein ARS2 |
| at | | | NO: 7 | |
| 219969 | 0.00097320 | CXorf15 | SEQ ID | chromosome X open reading frame 15 |
| at | | | NO: 98 | |

### Example 1A

The following example describes the identification of a biomarker panel that discriminates gefitinib-sensitive cell lines from gefitinib-resistant cell lines.

Methods: Gefitinib sensitivity was determined in 18 NSCLC cell lines using MTT assays. Cell lines were classified as gefitinib sensitive (IC₅₀<104), resistant (IC₅₀>10 μM) or intermediate sensitivity (10 μM<IC₅₀>1). Oligonucleotide gene arrays (Affymetrix® Human Genome U133 set, 39,000 genes) were done on 10 cell lines. Three distinct filtration and normalization algorithms to process the expression data were used, and a list of genes were generated that were both statistically significant (unadjusted p=0.001 cutoff) and corrected for false positive occurrence. This approach was used in combination with 5 distinct machine learning algorithms used to build a test set for predictor genes that were successful for 100% of the test cases. The best discriminators (>3 fold difference in expression between sensitive and resistant cell lines) were selected for Real-time RT-PCR.

Results: A list of genes was generated initially from the Affymetrix array analysis. By using the mathematical algorithm, 14 different candidate genes were selected for RT-PCR. Twelve of the 14 genes were verified to discriminate between sensitive and resistant cell lines by Real-time RT-PCR.

Conclusion: Based on NSCLC cell line studies it was possible to identify genes which strongly discriminated gefitinib (Iressa) sensitive cell lines from the resistant ones. The genes are ranked in Table 1A. This entire biomarker panel is of significant value for selecting NSCLC patients for gefitinib treatment.

TABLE 1A

| | | mean | mean | | | |
|---|---|---|---|---|---|---|
| Probe | parametric | intensity | intensity | Gene | Sequence | |
| set | p-value | (resistant) | (sensitive) | symbol | Identifier | Description |
| 202286 s | 0.00000005 | 3.8 | 9893.5 | TACSTD2 | SEQ ID | tumor-associated calcium |
| at | | | | | NO: 12 | signal transducer 2 |
| 202489_s_at | 0.00000005 | 25.8 | 2372.6 | FXYD3 | SEQ ID | FXYD domain containing |
| | | | | | NO: 16 | ion transport regulator 3 |
| 213285 | 0.00000005 | 8.0 | 1739.3 | TMEM30B | SEQ ID | transmembrane protein 30B |
| at | | | | | NO: 73 | |
| 218186 | 0.00000005 | 3.6 | 2295.0 | RAB25 | SEQ ID | RAB25, member RAS |
| at | | | | | NO: 83 | oncogene family |
| 235515 | 0.00000005 | 6.4 | 287.6 | FLJ36445 | SEQ ID | hypothetical protein |
| at | | | | | NO: 168 | FLJ36445 |
| 235988 | 0.00000005 | 11.3 | 345.7 | GPR110 | SEQ ID | G protein-coupled receptor |
| at | | | | | NO: 170 | 110 |
| 238689 | 0.00000005 | 5.4 | 2210.5 | GPR110 | SEQ ID | G protein-coupled receptor |
| at | | | | | NO: 177 | 110 |
| 232165 | 0.00000010 | 4.6 | 244.0 | EPPK1 | SEQ ID | epiplakin 1 |
| at | | | | | NO: 164 | |
| 240633 | 0.00000010 | 6.2 | 61.2 | FLJ33718 | SEQ ID | hypothetical protein |
| at | | | | | NO: 182 | FLJ33718 |
| 219525 | 0.00000020 | 179.3 | 6.1 | FLJ10847 | SEQ ID | hypothetical protein |
| at | | | | | NO: 93 | FLJ10847 |
| 229599_at | 0.00000020 | 5.9 | 112.8 | | SEQ ID | Clone IMAGE: 5166045, |
| | | | | | NO: 154 | Mrna |
| 203397_s_at | 0.00000030 | 10.1 | 1128.6 | GALNT3 | SEQ ID | UDP-N-acetyl-alpha-D- |
| | | | | | NO: 28 | galactosamine:polypeptide |
| | | | | | | N- |
| | | | | | | acetylgalactosaminyltransferase |
| | | | | | | 3 (GalNAc-T3) |
| 232164 s | 0.00000030 | 5.8 | 411.1 | EPPK1 | SEQ ID | epiplakin 1 |
| at | | | | | NO: 163 | |
| 212813 | 0.00000060 | 163.8 | 7.9 | JAM3 | SEQ ID | junctional adhesion |
| at | | | | | NO: 71 | molecule 3 |
| 227134 | 0.00000160 | 14.2 | 705.7 | SYTL1 | SEQ ID | synaptotagmin-like 1 |
| at | | | | | NO: 143 | |
| 236489 | 0.00000170 | 8.2 | 498.5 | | SEQ ID | |
| at | | | | | NO: 171 | |
| 235651 | 0.00000480 | 3.9 | 98.2 | | SEQ ID | |
| at | | | | | NO: 169 | |
| 238439 | 0.00000700 | 7.7 | 537.6 | ANKRD22 | SEQ ID | ankyrin repeat domain 22 |
| at | | | | | NO: 173 | |
| 219388 | 0.00000730 | 19.3 | 342.1 | TFCP2L3 | SEQ ID | transcription factor CP2- |
| at | | | | | NO: 91 | like 3 |
| 227985 | 0.00000820 | 5.0 | 179.9 | | SEQ ID | |
| at | | | | | NO: 146 | |
| 227450 | 0.00000890 | 5.1 | 509.7 | FLJ32115 | SEQ ID | hypothetical protein |
| at | | | | | NO: 144 | FLJ32115 |
| 203256 | 0.00000980 | 13.4 | 2223.0 | CDH3 | SEQ ID | cadherin 3, type 1, P- |
| at | | | | | NO: 23 | cadherin (placental) |
| 220318 | 0.00000980 | 4.4 | 44.7 | EPN3 | SEQ ID | epsin 3 |
| at | | | | | NO: 100 | |
| 202525 | 0.00001030 | 7.8 | 1194.6 | PRSS8 | SEQ ID | protease, serine, 8 |
| at | | | | | NO: 17 | (prostasin) |
| 227803_at | 0.00001080 | 7.8 | 206.1 | ENPP5 | SEQ ID | ectonudeotide |
| | | | | | NO: 145 | pyrophosphatase/phosphodiesterase |
| | | | | | | 5 (putative |
| | | | | | | function) |
| 206884 s | 0.00001200 | 12.8 | 822.7 | SCEL | SEQ ID | Sciellin |
| at | | | | | NO: 49 | |
| 223895 s | 0.00001290 | 13.8 | 183.6 | EPN3 | SEQ ID | epsin 3 |
| at | | | | | NO: 119 | |
| 238493 | 0.00001650 | 7.3 | 18.5 | ZNF506 | SEQ ID | zinc finger protein 506 |
| at | | | | | NO. 174 | |
| 224913 s | 0.00001960 | 2703.8 | 1081.5 | TIMM50 | SEQ ID | translocase of inner |
| at | | | | | NO: 122 | mitochondrial membrane |
| | | | | | | 50 homolog (yeast) |
| 201428 | 0.00002330 | 90.3 | 3416.4 | CLDN4 | SEQ ID | claudin 4 |
| at | | | | | NO: 5 | |
| 216641 s | 0.00003760 | 26.8 | 423.5 | LAD1 | SEQ ID | ladinin 1 |
| at | | | | | NO: 78 | |
| 231929_at | 0.00003910 | 31.0 | 340.7 | | SEQ ID | MRNA; cDNA |
| | | | | | NO: 159 | DKFZp586O0724 (from |
| | | | | | | clone DKFZp586O0724) |
| 212764_at | 0.00003930 | 320.0 | 9.2 | TCF8 | SEQ ID | transcription factor 8 |
| | | | | | NO: 70 | (represses interleukin 2 |
| | | | | | | expression) |
| 238778_at | 0.00004080 | 15.0 | 106.1 | MPP7 | SEQ ID | membrane protein, |
| | | | | | NO: 178 | palmitoylated 7 (MAGUK |
| | | | | | | p55 subfamily member 7) |
| 202641 | 0.00004360 | 2011.3 | 933.3 | ARL3 | SEQ ID | ADP-ribosylation factor- |
| at | | | | | NO: 19 | like 3 |
| 212233 | 0.00004550 | 2005.7 | 137.0 | MAP1B | SEQ ID | microtubule-associated |
| at | | | | | NO: 66 | protein 1B |
| 224232 s | 0.00004560 | 1054.1 | 438.3 | PX19 | SEQ ID | px19-like protein |
| at | | | | | NO: 120 | |
| 226905 | 0.00004590 | 240.2 | 14.0 | MGC45871 | SEQ ID | hypothetical protein |
| at | | | | | NO: 142 | MGC45871 |
| 218553_s_at | 0.00004620 | 177.0 | 38.2 | KCTD15 | SEQ ID | potassium channel |
| | | | | | NO: 84 | tetramerisation domain |
| | | | | | | containing 15 |
| 215218 s | 0.00004830 | 368.6 | 142.8 | C19orf14 | SEQ ID | chromosome 19 open |
| at | | | | | NO: 77 | reading frame 14 |
| 203287 | 0.00004920 | 23.4 | 505.0 | LAD1 | SEQ ID | ladinin 1 |
| at | | | | | NO: 24 | |
| 209114 | 0.00005560 | 43.7 | 717.2 | TSPAN-1 | SEQ ID | tetraspan 1 |
| at | | | | | NO: 57 | |
| 230076 | 0.00005660 | 21.2 | 120.1 | | SEQ ID | |
| at | | | | | NO: 155 | |
| 218677 | 0.00005710 | 21.5 | 966.3 | S100A14 | SEQ ID | S100 calcium binding |
| at | | | | | NO: 85 | protein A14 |
| 236616 | 0.00005810 | 17.8 | 32.9 | | SEQ ID | CDNA FLJ41623 fis, clone |
| at | | | | | NO: 172 | CTONG3009227 |
| 205014 | 0.00006280 | 13.4 | 491.2 | FGFBP1 | SEQ ID | fibroblast growth factor |
| at | | | | | NO: 40 | binding protein 1 |
| 200720_s_at | 0.00006360 | 1089.8 | 391.9 | ACTR1A | SEQ ID | ARP1 actin-related protein |
| | | | | | NO: 2 | 1 homolog A, centractin |
| | | | | | | alpha (yeast) |
| 224326 s | 0.00006750 | 499.6 | 135.5 | RNF134 | SEQ ID | ring finger protein 134 /// |
| at | | | | | NO: 121 | ring finger protein 134 |
| 242138 | 0.00006800 | 207.4 | 6.9 | DLX1 | SEQ ID | distal-less homeo box 1 |
| at | | | | | NO: 184 | |
| 90265 at | 0.00007110 | 145.0 | 1117.7 | CENTA1 | SEQ ID | centaurin, alpha 1 |
| | | | | | NO: 193 | |
| 222360 | 0.00007190 | 97.8 | 21.2 | CGI- | SEQ ID | CGI-30 protein |
| at | | | | 30 | NO: 108 | |
| 208393 s | 0.00007530 | 1370.0 | 596.5 | RAD50 | SEQ ID | RAD50 homolog (S. cerevisiae) |
| at | | | | | NO: 53 | |
| 226403 | 0.00007930 | 22.5 | 680.1 | TMC4 | SEQ ID | transmembrane channel- |
| at | | | | | NO: 136 | like 4 |
| 232056 | 0.00008450 | 9.8 | 141.7 | SCEL | SEQ ID | Scieliin |
| at | | | | | NO: 160 | |
| 207655 s | 0.00008700 | 7.1 | 71.1 | BLNK | SEQ ID | B-cell linker |
| at | | | | | NO: 51 | |
| 228683 s | 0.00009450 | 101.5 | 18.5 | KCTD15 | SEQ ID | potassium channel |
| at | | | | | NO: 148 | tetramerisation domain |
| | | | | | | containing 15 |
| 204160_s_at | 0.00009570 | 23.9 | 314.8 | ENPP4 | SEQ ID | Ectonucleotide |
| | | | | | NO: 36 | pyrophosphatase/phosphodiesterase |
| | | | | | | 4 (putative |
| | | | | | | function) |
| 202454_s_at | 0.00009860 | 16.3 | 1266.2 | ERBB3 | SEQ ID | v-erb-b2 erythroblastic |
| | | | | | NO: 15 | leukemia viral oncogene |
| | | | | | | homolog 3 (avian) |
| 232151_at | 0.00010020 | 8.5 | 295.7 | | SEQ ID | MRNA full length insert |
| | | | | | NO: 162 | cDNA clone |
| | | | | | | EUROIMAGE 2344436 |
| 205073_at | 0.00010350 | 30.8 | 136.8 | CYP2J2 | SEQ ID | cytochrome P450, family 2, |
| | | | | | NO: 41 | subfamily J, polypeptide 2 |
| 225658 | 0.00011660 | 167.1 | 516.3 | LOC339745 | SEQ ID | hypothetical protein |
| at | | | | | NO: 127 | LOC339745 |
| 219150 s | 0.00012240 | 30.9 | 200.1 | CENTA1 | SEQ ID | centaurin, alpha 1 |
| at | | | | | NO: 90 | |
| 228882 | 0.00012370 | 152.7 | 10.4 | TUB | SEQ ID | tubby homolog (mouse) |
| at | | | | | NO: 150 | |
| 222857_s_at | 0.00012430 | 17.2 | 344.7 | KCNMB4 | SEQ ID | potassium large |
| | | | | | NO: 113 | conductance calcium- |
| | | | | | | activated channel, |
| | | | | | | subfamily M, beta member 4 |
| 55662 at | 0.00013490 | 84.7 | 31.7 | C10orf76 | SEQ ID | chromosome 10 open |
| | | | | | NO: 191 | reading frame 76 |
| 204161_s_at | 0.00013900 | 12.5 | 69.3 | ENPP4 | SEQ ID | Ectonucleotide |
| | | | | | NO: 37 | pyrophosphatase/phosphodiesterase |
| | | | | | | 4 (putative |
| | | | | | | function) |
| 205455_at | 0.00014640 | 20.1 | 333.2 | MST1R | SEQ ID | macrophage stimulating 1 |
| | | | | | NO: 42 | receptor (c-met-related |
| | | | | | | tyrosine kinase) |
| 221432_s_at | 0.00014780 | 108.4 | 34.4 | SLC25A28 | SEQ ID | solute carrier family 25, |
| | | | | | NO: 102 | member 28 /// solute carrier |
| | | | | | | family 25, member 28 |
| 203082 | 0.00015630 | 1316.0 | 435.4 | BMS1L | SEQ ID | BMS1-like, ribosome |
| at | | | | | NO: 20 | assembly protein (yeast) |
| 223192 | 0.00015890 | 391.2 | 207.2 | SLC25A28 | SEQ ID | solute carrier family 25, |
| at | | | | | NO: 116 | member 28 |
| 226084 | 0.00017240 | 1660.7 | 87.5 | MAP1B | SEQ ID | microtubule-associated |
| at | | | | | NO: 131 | protein 1B |
| 229587 | 0.00017530 | 247.0 | 86.2 | UBA2 | SEQ ID | SUMO-1 activating |
| at | | | | | NO. 153 | enzyme subunit 2 |
| 211071_s_at | 0.00018080 | 2398.5 | 76.5 | AF1Q | SEQ ID | ALL1-fused gene from |
| | | | | | NO: 60 | chromosome 1q /// ALL1- |
| | | | | | | fused gene from |
| | | | | | | chromosome 1q |
| 214448_x_at | 0.00018290 | 310.0 | 123.8 | NFKBIB | SEQ ID | nuclear factor of kappa |
| | | | | | NO: 74 | light polypeptide gene |
| | | | | | | enhancer in B-cells |
| | | | | | | inhibitor, beta |
| 225413 | 0.00018660 | 8130.9 | 4324.6 | USMG5 | SEQ ID | upregulated during skeletal |
| at | | | | | NO: 125 | muscle growth 5 |
| 235036 | 0.00018930 | 262.2 | 19.4 | MGC46719 | SEQ ID | hypothetical protein |
| at | | | | | NO: 165 | MGC46719 |
| 203441 s | 0.00019180 | 684.0 | 72.1 | CDH2 | SEQ ID | cadherin 2, type 1, N- |
| at | | | | | NO: 31 | cadherin (neuronal) |
| 235247 | 0.00019200 | 6.2 | 262.8 | | SEQ ID | |
| at | | | | | NO: 167 | |
| 225096 | 0.00019610 | 1755.7 | 703.7 | HSA272196 | SEQ ID | hypothetical protein, clone |
| at | | | | | NO: 124 | 2746033 |
| 205617 | 0.00019960 | 9.2 | 23.1 | PRRG2 | SEQ ID | proline rich Gla (G- |
| at | | | | | NO: 44 | carboxyglutamic acid) 2 |
| 225822 | 0.00020110 | 10.3 | 468.3 | MGC17299 | SEQ ID | hypothetical protein |
| at | | | | | NO: 129 | MGC17299 |
| 239077 | 0.00020310 | 146.8 | 49.3 | GALNACT-2 | SEQ ID | chondroitin sulfate |
| at | | | | | NO: 179 | GalNAcT-2 |
| 218779 x | 0.00021870 | 72.0 | 404.0 | EPS8L1 | SEQ ID | EPS8-like 1 |
| at | | | | | NO: 86 | |
| 50314 i | 0.00022630 | 830.5 | 279.4 | C20orf27 | SEQ ID | chromosome 20 open |
| at | | | | | NO: 190 | reading frame 27 |
| 218792 s | 0.00023140 | 74.9 | 468.6 | BSPRY | SEQ ID | B-box and SPRY domain |
| at | | | | | NO: 87 | containing |
| 222664_at | 0.00024210 | 624.9 | 42.5 | KCTD15 | SEQ ID | potassium channel |
| | | | | | NO: 109 | tetramerisation domain |
| | | | | | | containing 15 |
| 201869 s | 0.00024250 | 290.8 | 70.5 | TBL1X | SEQ ID | transducin (beta)-like 1X- |
| at | | | | | NO: 9 | linked |
| 219855_at | 0.00024820 | 233.0 | 27.6 | NUDT11 | SEQ ID | nudix (nucleoside |
| | | | | | NO: 94 | diphosphate linked moiety |
| | | | | | | X)-type motif |
| 203236_s_at | 0.00025890 | 81.3 | 318.7 | LGALS9 | SEQ ID | 11 lectin, galactoside- |
| | | | | | NO: 22 | binding, soluble, 9 (galectin |
| | | | | | | 9) |
| 202167 s | 0.00026530 | 770.6 | 340.7 | MMS19L | SEQ ID | MMS19-like (MET18 |
| at | | | | | NO: 10 | homolog, S. cerevisiae) |
| 229223 | 0.00026990 | 21.7 | 130.8 | | SEQ ID | |
| at | | | | | NO: 152 | |
| 201157 s | 0.00027160 | 2272.3 | 1323.6 | NMT1 | SEQ ID | N-myristoyltransferase 1 |
| at | | | | | NO: 4 | |
| 226187_at | 0.00027300 | 32.2 | 301.2 | CDS1 | SEQ ID | CDP-diacylglycerol |
| | | | | | NO: 132 | synthase (phosphatidate |
| | | | | | | cytidylyltransferase) 1 |
| 239671 | 0.00028050 | 12.2 | 43.6 | | SEQ ID | CDNA FLJ31085 fis, clone |
| at | | | | | NO: 181 | IMR321000037 |
| 222746 s | 0.00028540 | 8.7 | 288.5 | BSPRY | SEQ ID | B-box and SPRY domain |
| at | | | | | NO: 111 | containing |
| 219858 s | 0.00029160 | 12.3 | 63.1 | FLJ20160 | SEQ ID | FLJ20160 protein |
| at | | | | | NO: 96 | |
| 210749 x | 0.00029280 | 507.7 | 2452.9 | DDR1 | SEQ ID | discoidin domain receptor |
| at | | | | | NO: 59 | family, member 1 |
| 211778 s | 0.00029620 | 20.3 | 334.6 | ZNF339 | SEQ ID | zinc finger protein 339 /// |
| at | | | | | NO: 61 | zinc finger protein 339 |
| 226876 | 0.00030570 | 283.5 | 45.7 | MGC45871 | SEQ ID | hypothetical protein |
| at | | | | | NO: 141 | MGC45871 |
| 230323 s | 0.00033140 | 17.4 | 295.5 | LOC120224 | SEQ ID | hypothetical protein |
| at | | | | | NO: 157 | BC016153 |
| 221665 s | 0.00033480 | 20.5 | 172.5 | EPS8L1 | SEQ ID | EPS8-like 1 |
| at | | | | | NO: 105 | |
| 1007 s at | 0.00033840 | 469.2 | 2729.2 | DDR1 | SEQ ID | discoidin domain receptor |
| | | | | | NO: 1 | family, member 1 |
| 218891 | 0.00034090 | 218.3 | 108.6 | C10orf76 | SEQ ID | chromosome 10 open |
| at | | | | | NO: 88 | reading frame 76 |
| 218960 | 0.00034100 | 25.7 | 408.5 | TMPRSS4 | SEQ ID | transmembrane protease, |
| at | | | | | NO: 89 | serine 4 |
| 222668_at | 0.00034910 | 573.0 | 38.2 | KCTD15 | SEQ ID | potassium channel |
| | | | | | NO: 110 | tetramerisation domain |
| | | | | | | containing 15 |
| 217496 s | 0.00036040 | 593.8 | 172.2 | IDE | SEQ ID | insulin-degrading enzyme |
| at | | | | | NO: 81 | |
| 226213 | 0.00036180 | 27.4 | 1639.9 | ERBB3 | SEQ ID | v-erb-b2 erythroblastic |
| at | | | | | NO: 133 | leukemia viral oncogene |
| | | | | | | homolog 3 (avian) |
| 235202 x | 0.00036460 | 59.3 | 14.9 | [KIP | SEQ ID | IKK interacting protein |
| at | | | | | NO: 166 | |
| 212736 | 0.00036600 | 290.0 | 27.4 | BC008967 | SEQ ID | hypothetical gene |
| at | | | | | NO: 69 | BC008967 |
| 203327 | 0.00036980 | 410.7 | 105.9 | IDE | SEQ ID | insulin-degrading enzyme |
| at | | | | | NO: 26 | |
| 202597 | 0.00037880 | 5.1 | 129.6 | IRF6 | SEQ ID | interferon regulatory factor 6 |
| at | | | | | NO: 18 | |
| 228865 | 0.00037970 | 9.2 | 322.3 | SARG | SEQ ID | specifically androgen- |
| at | | | | | NO: 149 | regulated protein |
| 205709_s_at | 0.00038120 | 13.4 | 254.3 | CDS1 | SEQ ID | CDP-diacylglycerol |
| | | | | | NO: 45 | synthase (phosphatidate |
| | | | | | | cytidylyltransferase) 1 |
| 224946 s | 0.00039420 | 329.1 | 681.4 | MGC12981 | SEQ ID | hypothetical protein |
| at | | | | | NO: 123 | MGC12981 |
| 204856_at | 0.00039710 | 80.7 | 400.7 | B3GNT3 | SEQ ID | UDP-GlcNAc:betaGal |
| | | | | | NO: 39 | beta-1,3-N- |
| | | | | | | acetylglucosaminyltransferase 3 |
| 203317 | 0.00039900 | 58.0 | 171.0 | PSD4 | SEQ ID | pleckstrin and Sec7 domain |
| at | | | | | NO: 25 | containing 4 |
| 221958 s | 0.00040170 | 171.2 | 468.6 | FLJ23091 | SEQ ID | putative NFkB activating |
| at | | | | | NO: 106 | protein 373 |
| 201130 s | 0.00040570 | 15.3 | 1183.0 | CDH1 | SEQ ID | cadherin 1, type 1, E- |
| at | | | | | NO: 3 | cadherin (epithelial) |
| 205458_at | 0.00042200 | 109.4 | 57.6 | MC1R | SEQ ID | melanocortin 1 receptor |
| | | | | | NO: 43 | (alpha melanocyte |
| | | | | | | stimulating hormone |
| | | | | | | receptor) |
| 205847 | 0.00042390 | 71.8 | 206.0 | PRSS22 | SEQ ID | protease, serine, 22 |
| at | | | | | NO: 47 | |
| 202340_x_at | 0.00043030 | 336.4 | 72.7 | NR4A1 | SEQ ID | nuclear receptor subfamily |
| | | | | | NO: 14 | 4, group A, member 1 |
| 215146 s | 0.00043080 | 165.6 | 48.8 | KIAA1043 | SEQ ID | KIAA1043 protein |
| at | | | | | NO: 76 | |
| 223032 x | 0.00043320 | 5068.6 | 2903.7 | PX19 | SEQ ID | px19-like protein |
| at | | | | | NO: 115 | |
| 226535 | 0.00044520 | 15.3 | 862.3 | ITGB6 | SEQ ID | integrin, beta 6 |
| at | | | | | NO: 137 | |
| 65517 at | 0.00045130 | 50.8 | 387.0 | AP1M2 | SEQ ID | adaptor-related protein |
| | | | | | NO: 192 | complex 1, mu 2 subunit |
| 91826 at | 0.00045430 | 59.7 | 373.3 | EPS8L1 | SEQ ID | EPS8-like 1 |
| | | | | | NO: 194 | |
| 238673 | 0.00045640 | 44.3 | 578.2 | | SEQ ID | |
| at | | | | | NO: 176 | |
| 221610 s | 0.00046860 | 83.5 | 569.8 | STAP2 | SEQ ID | signal-transducing adaptor |
| at | | | | | NO: 104 | protein-2 |
| 203779 s | 0.00047400 | 17.8 | 143.2 | EVA1 | SEQ ID | epithelial V-like antigen 1 |
| at | | | | | NO: 33 | |
| 230312 | 0.00047560 | 91.2 | 11.6 | | SEQ ID | |
| at | | | | | NO: 156 | |
| 211855_s_at | 0.00047620 | 355.5 | 97.2 | SLC25A14 | SEQ ID | solute carrier family 25 |
| | | | | | NO: 62 | (mitochondrial carrier, |
| | | | | | | brain), member 14 |
| 222830 | 0.00047770 | 31.3 | 586.6 | TFCP2L2 | SEQ ID | transcription factor CP2- |
| at | | | | | NO. H2 | like 2 |
| 203780 | 0.00047790 | 33.5 | 647.3 | EVA1 | SEQ ID | epithelial V-like antigen 1 |
| at | | | | | NO: 34 | |
| 223233 s | 0.00048700 | 37.9 | 541.0 | CGN | SEQ ID | cingulin |
| at | | | | | NO: 117 | |
| 219412 | 0.00049410 | 6.2 | 241.9 | RAB38 | SEQ ID | RAB38, member RAS |
| at | | | | | NO: 92 | oncogene family |
| 219936 s | 0.00049770 | 5.8 | 171.1 | GPR87 | SEQ ID | G protein-coupled receptor |
| at | | | | | NO: 97 | 87 |
| 226226 | 0.00049820 | 31.5 | 465.5 | LOC120224 | SEQ ID | hypothetical protein |
| at | | | | | NO: 134 | BC016153 |
| 222280 | 0.00050070 | 312.5 | 152.0 | | SEQ ID | CDNA clone |
| at | | | | | NO: 107 | IMAGE: 6602785, partial |
| | | | | | | cds |
| 225911 | 0.00050990 | 6.9 | 142.2 | LOC255743 | SEQ ID | hypothetical protein |
| at | | | | | NO: 130 | LOC255743 |
| 223295 s | 0.00053580 | 463.2 | 264.9 | LUC7L | SEQ ID | LUC7-like (S. cerevisiae) |
| at | | | | | NO: 118 | |
| 212120 | 0.00053760 | 1118.9 | 381.7 | RHOQ | SEQ ID | ras homolog gene family, |
| at | | | | | NO: 65 | member Q |
| 226584 s | 0.00053900 | 81.8 | 186.8 | C20orf55 | SEQ ID | chromosome 20 open |
| at | | | | | NO: 138 | reading frame 55 |
| 202328_s_at | 0.00054270 | 307.4 | 127.3 | PKD1 | SEQ ID | polycystic kidney disease 1 |
| | | | | | NO: 13 | (autosomal dominant) |
| 208779 x | 0.00054830 | 489.8 | 2385.8 | DDR1 | SEQ ID | discoidin domain receptor |
| at | | | | | NO: 55 | family, member 1 |
| 203783 x | 0.00055660 | 33.6 | 14.8 | POLRMT | SEQ ID | polymerase (RNA) |
| at | | | | | NO: 35 | mitochondria! (DNA |
| | | | | | | directed) |
| 208084 | 0.00055660 | 29.0 | 347.8 | ITGB6 | SEQ ID | integrin, beta 6 |
| at | | | | | NO: 52 | |
| 213262 | 0.00056350 | 597.1 | 48.5 | SACS | SEQ ID | spastic ataxia of |
| at | | | | | NO: 72 | Charlevoix-Saguenay |
| | | | | | | (sacsin) |
| 225793 | 0.00058010 | 1662.4 | 133.4 | MGC46719 | SEQ ID | hypothetical protein |
| at | | | | | NO: 128 | MGC46719 |
| 226678 | 0.00058120 | 63.1 | 171.9 | UNC13D | SEQ ID | unc-13 homolog D (C. elegans) |
| at | | | | | NO: 139 | |
| 216949_s_at | 0.00058240 | 83.3 | 27.2 | PKD1 | SEQ ID | polycystic kidney disease 1 |
| | | | | | NO: 80 | (autosomal dominant) |
| 212338 | 0.00058710 | 28.0 | 335.5 | MYO1D | SEQ ID | myosin ID |
| at | | | | | NO: 67 | |
| 241455 | 0.00059440 | 7.3 | 68.8 | | SEQ ID | |
| at | | | | | NO: 183 | |
| 214577 | 0.00062040 | 279.3 | 58.3 | MAP1B | SEQ ID | microtubule-associated |
| at | | | | | NO: 75 | protein 1B |
| 220178 | 0.00062110 | 193.7 | 48.8 | C19orf28 | SEQ ID | chromosome 19 open |
| at | | | | | NO: 99 | reading frame 28 |
| 201868 s | 0.00062220 | 103.1 | 21.6 | TBL1X | SEQ ID | transducin (beta)-like 1X- |
| at | | | | | NO: 8 | linked |
| 201679 | 0.00063150 | 451.3 | 212.9 | ARS2 | SEQ ID | arsenate resistance protein |
| at | | | | | NO: 6 | ARS2 |
| 206043 s | 0.00063910 | 8.0 | 67.9 | KIAA0703 | SEQ ID | KIAA0703 gene product |
| at | | | | | NO: 48 | |
| 226706 | 0.00063930 | 81.4 | 847.1 | FLJ23867 | SEQ ID | hypothetical protein |
| at | | | | | NO: 140 | FLJ23867 |
| 210255 | 0.00064190 | 8.8 | 36.1 | RAD51L1 | SEQ ID | RAD51-like 1 (S. cerevisiae) |
| at | | | | | NO: 58 | |
| 208968 s | 0.00066500 | 2065.0 | 1181.4 | CIAPIN1 | SEQ ID | cytokine induced apoptosis |
| at | | | | | NO: 56 | inhibitor 1 |
| 207627 s | 0.00068160 | 401.7 | 205.1 | TFCP2 | SEQ ID | transcription factor CP2 |
| at | | | | | NO: 50 | |
| 203407 | 0.00068500 | 39.6 | 1680.0 | PPL | SEQ ID | periplakin |
| at | | | | | NO: 29 | |
| 217791 s | 0.00069580 | 1777.8 | 837.7 | ALDH18A1 | SEQ ID | aldehyde dehydrogenase 18 |
| at | | | | | NO: 82 | family, member A1 |
| 225582 | 0.00069740 | 415.9 | 44.7 | KIAA1754 | SEQ ID | KIAA1754 |
| at | | | | | NO: 126 | |
| 231721 | 0.00070410 | 37.7 | 4.4 | JAM3 | SEQ ID | junctional adhesion |
| at | | | | | NO: 158 | molecule 3 |
| 222859_s_at | 0.00072460 | 24.0 | 133.1 | DAPP1 | SEQ ID | dual adaptor of |
| | | | | | NO: 114 | phosphotyrosine and 3- |
| | | | | | | phosphoinositides |
| 208595 s | 0.00074160 | 263.9 | 122.8 | MBD1 | SEQ ID | methyl-CpG binding |
| at | | | | | NO: 54 | domain protein 1 |
| 212015 | 0.00075720 | 5744.3 | 3435.4 | PTBP1 | SEQ ID | polypyrimidine tract |
| X at | | | | | NO: 63 | binding protein 1 |
| 219856 | 0.00075780 | 13.9 | 230.4 | SARG | SEQ ID | specifically androgen- |
| at | | | | | NO: 95 | regulated protein |
| 38766 at | 0.00075940 | 85.9 | 281.7 | SRCAP | SEQ ID | Snf2-related CBP activator |
| | | | | | NO: 189 | protein |
| P204744 | 0.00076150 | 7537.7 | 3827.7 | IARS | SEQ ID | isoleucine-tRNA synthetase |
| s at | | | | | NO: 38 | |
| 239196 | 0.00076210 | 30.5 | 550.5 | ANKRD22 | SEQ ID | ankyrin repeat domain 22 |
| at | | | | | NO: 180 | |
| 203718 | 0.00076760 | 424.0 | 138.4 | NTE | SEQ ID | neuropathy target esterase |
| at | | | | | NO: 32 | |
| 232149_s_at | 0.00076810 | 414.2 | 127.6 | NSMAF | SEQ ID | neutral sphingomyelinase |
| | | | | | NO: 161 | (N-SMase) activation |
| | | | | | | associated factor |
| 202264_s_at | 0.00076920 | 1513.7 | 830.7 | TOMM40 | SEQ ID | translocase of outer |
| | | | | | NO: 11 | mitochondrial membrane |
| | | | | | | 40 homolog (yeast) |
| 32069 at | 0.00077000 | 147.8 | 266.2 | N4BP1 | SEQ ID | Nedd4 binding protein 1 |
| | | | | | NO: 187 | |
| 216862 s | 0.00078160 | 901.3 | 359.6 | MTCP1 | SEQ ID | mature T-cell proliferation 1 |
| at | | | | | NO: 79 | |
| 220370 s | 0.00079540 | 306.1 | 60.5 | USP36 | SEQ ID | ubiquitin specific protease |
| at | | | | | NO: 101 | 36 |
| 242191 | 0.00080180 | 152.0 | 35.5 | | SEQ ID | LOC400781 |
| at | | | | | NO: 185 | |
| 203109_at | 0.00081840 | 2445.5 | 1097.7 | UBE2M | SEQ ID | ubiquitin-conjugating |
| | | | | | NO: 21 | enzyme E2M (UBC12 |
| | | | | | | homolog, yeast) |
| 205780 | 0.00083050 | 39.8 | 941.1 | | SEQ ID | |
| at | | | | | NO: 46 | |
| 203440 | 0.00\083250 | 503.5 | 78.6 | CDH2 | SEQ ID | cadherin 2, type 1, N- |
| at | | | | | NO: 30 | cadherin (neuronal) |
| 238513_at | 0.00083510 | 73.6 | 618.6 | TMG4 | SEQ ID | transmembrane gamma- |
| | | | | | NO: 175 | carboxyglutamic acid |
| | | | | | | protein 4 |
| 221550_at | 0.00083680 | 414.1 | 200.9 | COX15 | SEQ ID | COX15 homolog, |
| | | | | | NO: 103 | cytochrome c oxidase |
| | | | | | | assembly protein (yeast) |
| 229030 | 0.00084650 | 5.9 | 70.1 | | SEQ ID | |
| at | | | | | NO: 151 | |
| 226400 | 0.00088590 | 2284.5 | 4256.7 | | SEQ ID | |
| at | | | | | NO: 135 | |
| 37966 at | 0.00090730 | 127.8 | 9.3 | PARVB | SEQ ID | parvin, beta |
| | | | | | NO: 188 | |
| 212424 | 0.00092430 | 381.6 | 115.2 | PDCD11 | SEQ ID | programmed cell death 11 |
| at | | | | | NO: 68 | |
| 228441 s | 0.00093570 | 12.0 | 49.8 | | SEQ ID | |
| at | | | | | NO: 147 | |
| 203328 x | 0.00095810 | 411.3 | 112.2 | IDE | SEQ ID | insulin-degrading enzyme |
| at | | | | | NO: 27 | |
| 201680 x | 0.00095980 | 1383.3 | 765.5 | ARS2 | SEQ ID | arsenate resistance protein |
| at | | | | | NO: 7 | ARS2 |
| 243302 | 0.00096750 | 14.2 | 29.1 | | SEQ ID | |
| at | | | | | NO: 186 | |
| 219969 | 0.00097320 | 102.8 | 21.4 | CXorf15 | SEQ ID | chromosome X open |
| at | | | | | NO: 98 | reading frame 15 |
| 212016 s | 0.00099210 | 4187.6 | 2276.0 | PTBP1 | SEQ ID | polypyrimidine tract |
| at | | | | | NO: 64 | binding protein 1 |

### Example 1B

The following example describes the identification of a biomarker panel that discriminates erlotinib-sensitive cell lines from erlotinib-resistant cell lines.

Methods: Erlotinib sensitivity is determined in 18 NSCLC cell lines using MTT assays. Cell lines are classified as erlotinib sensitive (IC₅₀<1 μM), resistant (IC₅₀>10 μM) or intermediate sensitivity (10 μM<IC₅₀>1). Oligonucleotide gene arrays (Affymetrix® Human Genome U133 set, 39,000 genes) are done on 10 cell lines. Three distinct filtration and normalization algorithms to process the expression data are used, and a list of genes are generated that are both statistically significant (unadjusted p=0.001 cutoff) and corrected for false positive occurrence. This approach is used in combination with 5 distinct machine learning algorithms used to build a test set for predictor genes that are successful for 100% of the test cases. The best discriminators (>3 fold difference in expression between sensitive and resistant cell lines) are selected for Real-time RT-PCR.

Results: A list of genes is generated initially from the Affymetrix array analysis. By using the mathematical algorithm, 10-20 different candidate genes are selected for RT-PCR.

Conclusion: Based on NSCLC cell line studies it is possible to identify genes which strongly discriminate erlotinib sensitive cell lines from the resistant ones.

### Example 1C

The following example describes the identification of a biomarker panel that discriminates lapatinib-sensitive cell lines from lapatinib-resistant cell lines.

Methods: Lapatinib sensitivity is determined in 18 NSCLC cell lines using MTT assays. Cell lines are classified as lapatinib sensitive (IC₅₀<1 μM), resistant (IC₅₀>10 μM) or intermediate sensitivity (10 μM<IC₅₀>1). Oligonucleotide gene arrays (Affymetrix® Human Genome U133 set, 39,000 genes) are done on 10 cell lines. Three distinct filtration and normalization algorithms to process the expression data are used, and a list of genes are generated that are both statistically significant (unadjusted p=0.001 cutoff) and corrected for false positive occurrence. This approach is used in combination with 5 distinct machine learning algorithms used to build a test set for predictor genes that are successful for 100% of the test cases. The best discriminators (>3 fold difference in expression between sensitive and resistant cell lines) are selected for Real-time RT-PCR.

Results: A list of genes is generated initially from the Affymetrix array analysis. By using the mathematical algorithm, 10-20 different candidate genes are selected for RT-PCR.

Conclusion: Based on NSCLC cell line studies it is possible to identify genes which strongly discriminate lapatinib sensitive cell lines from the resistant ones.

### Example 2

The following example describes the identification and further investigation of a target gene identified using the gene expression profile disclosed herein.

In this experiment, the present inventors describe research to examine the influence of E-cadherin-regulatory molecules on non-small cell lung cancer (NSCLC) response to EGF receptor (EGFR) inhibitors.

The EGFR, a member of the erbB family of tyrosine kinases (erbB1-4) plays a major role in transmitting stimuli that lead to NSCLC cellular proliferation and survival. EGFR, highly expressed in NSCLC, is a primary target for NSCLC therapeutic intervention. In clinical trials, 11-20% of patients with chemo-refractory advanced stage NSCLC responded to treatment with EGFR inhibitors such as gefitinib (Iressa®, ZD1839). Currently, there are no markers that predict which patients will respond to treatment. NSCLC patients with poor survival have decreased expression of E-cadherin, a cell adhesion molecule. E-cadherin expression is regulated by the wnt pathway and by zinc finger transcription factors including δEF1/ZEB1 and SIP1/ZEB2. Higher levels of protein expression of E-cadherin were detected in gefitinib sensitive NSCLC cell lines and expression was absent in gefitinib resistant lines. Conversely, expression of the E-cadherin inhibitors ZEB1 and SEP1 was higher in gefitinib resistant cell lines. The hypothesis of this project is that expression of E-cadherin and its regulatory molecules predict response to EGFR inhibitors, and modulating E-cadherin regulatory proteins may augment response to EGFR inhibitors in non-small cell lung cancer.

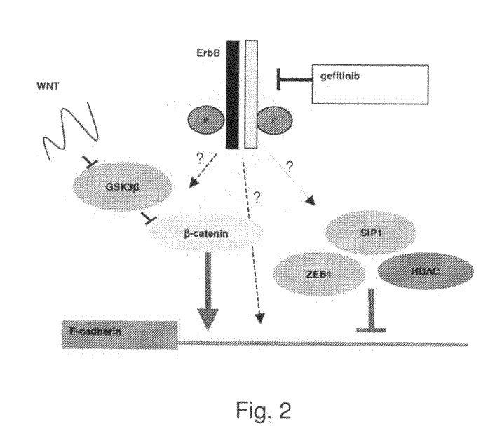

E-cadherin, a calcium-dependent epithelial cell adhesion molecule, plays an important role in tumor invasiveness and metastatic potential. Reduced E-cadherin expression is associated with tumor cell dedifferentiation, advanced stage and reduced survival in patients with NSCLC. At the transcriptional level, the wnt/β-catenin signaling pathway regulates DE-cadherin expression. The present inventors have reported that inhibition of GSK313, involved in the proteasomal degradation of β-catenin, lead E-cadherin upregulation (FIG. 2). E-cadherin transcription is also regulated by zinc finger transcription factors including, Snail, Slug, ZEB1 and SIP1. They repress E-cadherin expression by binding to its promoter and recruiting HDAC (FIG. 2). The inventors have reported that inhibiting the ZEB1 or HDAC expression lead to upregulation of E-cadherin in NSCLC cell lines.

In this experiment, the inventors used NSCLC cell lines to: (1) evaluate the growth inhibitory properties of EGFR inhibitors by MTT assays, (2) to identify molecular molecules through DNA microarrays and westerns that predict response to EGFR inhibitors and (3) to design combination therapies that enhance the effect of the EGFR inhibitors. Cell lines were screened for expression of members of the EGFR and Wnt signaling pathways. E-cadherin expression was found to be lacking in gefitinib resistant cell lines and activated in gefitinib sensitive lines. Therefore, the expression of zinc finger transcription factors involved in E-cadherin repression was investigated. It was determined that gefitinib resistant lines have high ZEB1 and/or SIP1 expression, and expression is lacking in gefitinib-sensitive lines.

The inventors proposed that SIP1 and ZEB1 expression predicts EGFR tyrosine kinase inhibitors resistance and that modulating the molecular mechanism that regulate E-cadherin expression will enhance sensitivity to EGFR inhibitors. The proposal will be tested by manipulating E-cadherin expression and measuring the effect on response to gefitinib. Results of this work will be evaluated in clinical trials in patients with NSCLC.

### Results

EGFR. pEGFR. Her2. ErbB3 and Erb4 Expression in NSCLC:

EGFR, Her-2 and ErbB3 cell surface expression was evaluated using flow cytometry (Table 2). The majority of NSCLC cell lines (15/18) had a high percentage of EGFR positive cells and three had low or no EGFR expression. The two BAC cell lines, H322 and H358, had high expression of EGFR and Her2.

TABLE 2

| | FACS | FACS | FACS | |
|---|---|---|---|---|
| | % EGFR/ | % Her2/ | % ErbB3/ | IC 50 uM |
| Cell Line | MFI | MFI | MFI | ZD 1839 |
| Adenocarcinoma | | | | |
| Calu3 | 98%/8.9 | 100/37 | 32/4.3 | <1 |
| Colo699 | 0/0 | 0/0 | 57/2.3 | 4.1 |
| H125 | 100/34 | 91/2.8 | 0/0 | 4.7 |
| H2122 | 94/5.1 | 73/4 | 80/5 | 4.8 |
| H1435 | 98/14 | ND | 94/6.4 | 7.6 |
| A549 | 99/14 | 72/2.4 | 54/3.5 | 8.4 |
| H441 | 78/6.9 | 79/2.6 | 0/0 | 11.7 |
| HI 648 | 98/5.7 | 78/2.7 | 0/0 | 11.5 |
| Bronchoalveolar | | | | |
| H322 | 100/16 | 96.5.5 | ND | <1 |
| H358 | ND | ND | ND | <1 |
| Squamous Cell | | | | |
| NE18 | 100/16 | 98/3.3 | 35/5.7 | 8 |
| H1703 | 99/15 | 65/2.6 | 0/0 | 9.3 |
| H157 | 93/13 | 62/1.8 | 0/0 | 10.1 |
| H520 | 0/0 | 0/0 | 0/0 | 10.3 |
| H1264 | 100/14 | 43/1.9 | 0/0 | 10.2 |
| Large Cell | | | | |
| H1334 | 100/23 | 74/3.2 | 99/10 | 3.8 |
| H460 | 37/1.9 | 57/1.4 | 0/0 | 9/9 |

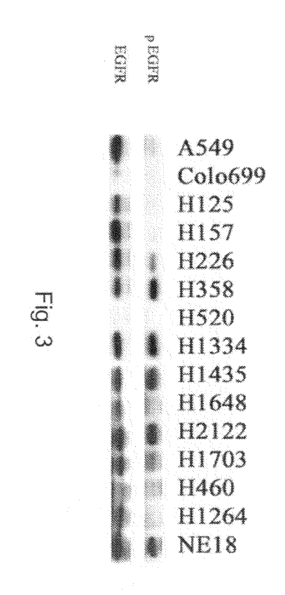

The presence of phosphorylated EGFR (pEGFR) versus EGFR was evaluated by Western blotting in 18 NSCLC cell lines (FIG. 3, shows 15 cell lines). EGFR was detected in the majority of NSCLC cell lines, whereas only a subset of these cell lines had (pEGFR).

### Effects of EGFR Inhibitors on Human Lung Cancer Cells Growth:

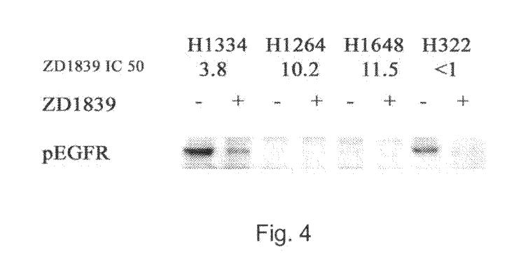

The growth inhibitory effect of gefitinib, on 18 NSCLC cell lines was evaluated using the MTT assay (Table 2). There was no correlation between the EGFR expression and gefitinib response. The change in pEGFR following gefitinib treatment was evaluated in two sensitive cell lines, H1334 and H322, and two resistant cell lines, H1264 and H1648 (FIG. 4). Gefitinib inhibited the phosphorylated “active” form of EGFR in sensitive cell lines.

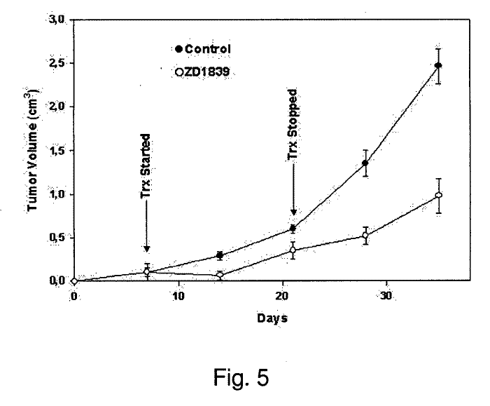

Based on the in vitro experiments, athymic nude mice bearing human NSCLC xenografts were treated with EGFR inhibitors ZD1839 or C225. Growth delay was evident in tumors after treatment with either agent (FIG. 5).

### E-Cadherin, SIP1 and ZEB1 in NSCLC Cell Lines Using Microarray and RT-PCR and Western Blotting:

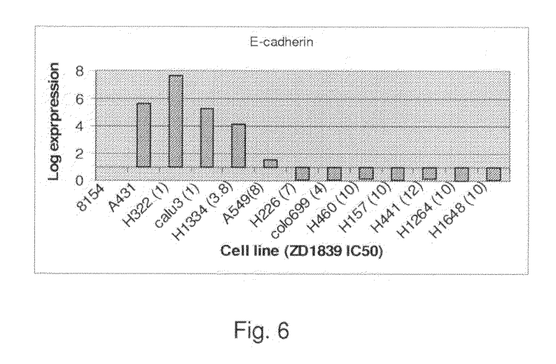

High density oligonucleotide microarray (IOAM) analysis of gene expression levels of selected genes was developed from 11 NSCLC cell lines. These cell lines included 2 gefitinib sensitive lines (IC₅₀<1 μM), 5 gefitinib resistant lines (IC₅₀>1 μM), and 4 lines with intermediate sensitivity (IC₅₀>1 μM, <10 μM). The expression of E-cadherin, SIP1 and ZEB1 was evaluated and compared to their expression in normal bronchial epithelium using the Gene Spring program (FIG. 6).

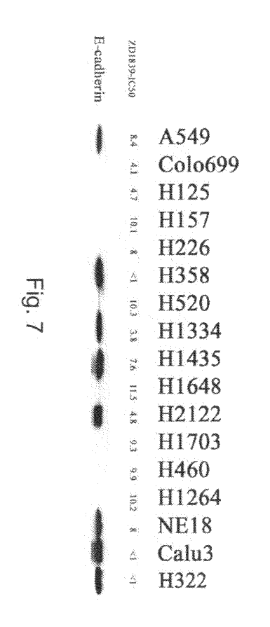

E-cadherin expression was more pronounced in gefitinib sensitive lines and absent in gefitinib resistant lines. This expression pattern was confirmed using western blotting and real time PCR (RT-PCR) (FIG. 7).

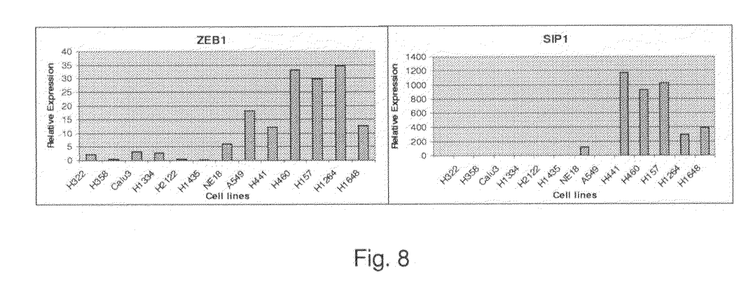

As discussed above, regulation of E-cadherin expression involves the zinc finger transcription factors ZEB1 and SIP1. Expression of both transcription factors was evaluated using real time RT-PCR. ZEB1 and SIP1 were expressed in the gefitinib resistant lines and absent in the gefitinib sensitive lines (FIG. 8). The expression of Slug, Snail, Wnt7a, β-catenin, γ-catenin, α-catenin and GSK3β was also evaluated using Western blot analysis or RT-PCR. None of theses molecules had a differential pattern of expression in the NSCLC lines (data not shown).

In summary, there was no correlation between gefitinib sensitivity and EGFR expression. E-cadherin was detected preferentially in gefitinib sensitive lines.

Conversely, the zinc finger transcription factors, ZEB1 and SIP1, involved in E-cadherin inhibition were expressed in gefitinib resistant lines and absent in gefitinib sensitive lines.

### Example 3

This example describes the evaluation of the detrimental effect of the zinc finger proteins ZEB1 and SIP1 on NSCLC cell lines sensitivity to EGFR inhibitors.

In the first part of this experiment, adenoviral constructs containing ZEB1 or SIP1 are used to overexpress these proteins in gefitinib sensitive cell lines. MTT assay will assess changes in gefitinib sensitivity. In the second part of this experiment, stably transfected ZEB1 and SIP1 cell lines and untransfected cell lines are implanted into nude mice. Transplanted mice are treated with gefitinib and the response is compared between the two groups.

### Example 4

This example describes the determination of the molecular mechanisms that improve the response to EGFR inhibitors in NSCLC cell lines in vitro and in vivo.

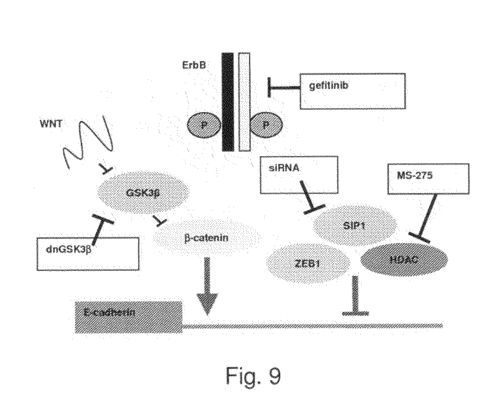

In the first part of this experiment, the effect of “silencing” the E-cadherin transcriptional repressors, SIP1 and ZEB1, on NSCLC cell lines response to ZD1839 is examined. To directly examine the role of the zinc-finger transcription factors, SIP1 and ZEB1 on gefitinib responsive lines, the effect of siRNA is developed and tested (FIG. 9). siRNA is prepared for different regions of SIP1 and ZEB1 using the silencer kit from Dharmacon (Colorado). Their efficacy is tested by RT-PCR. The most effective siRNA for SIP1 and ZEB1 are then introduced, individually or in combination, into gefitinib resistant lines. The effect of these siRNAs on gefitinib responsiveness is evaluated by MTT assay. ZEB1 antibody (Santa Cruz, Calif.) and SIP1 antibody (a gift from Dr. van Grunsven) are used to evaluate the efficacy of RNA inhibition.

In the second part of this experiment, the effect of inhibiting GSK3β on gefitinib response in NSCLC cell lines is examined. GSK3β phosphorylates β-catenin leading to its ubiquitination and destruction. GSK3β inhibitors, such as lithium, increased E-cadherin expression in NSCLC cell lines. GSK3β function is inhibited with an adenovirus (pAdTrack-CMV) encoding a dominant-negative GSK3β (dnGSK3β). To determine the effectiveness of this dnGSKβ the expression of non-phosphorylated β-catenin and E-cadherin is evaluated by western blot. NSCLC cell lines stably transfected with the dnGSK3β construct are generated. The effect of inhibiting GSK3β on NSCLC cell lines response to gefitinib are evaluated using MTT assays.

In the third part of this experiment, the effect of E-cadherin on gefitinib sensitivity is evaluated. Resistant NSCLC lines are transfected with E-cadherin encoding constructs. Changes in NSCLC cell lines response to gefitinib are assessed by MTT assay. Gefitinib-sensitive lines that express E-cadherin are treated with an E-cadherin antibody (Zymed) and the effect on gefitinib responsiveness assessed by MTT assay. The results determine whether expression of E-cadherin itself is sufficient to determine gefitinib sensitivity, or if sensitivity is a reflexion of events occurring upstream of it.

In the fourth part of this experiment, the effect of gefitinib responsiveness on NSCLC cell lines is augmented in vivo. Based on findings from the above in vitro experiments, the best treatment that enhances gefitinib sensitivity in NSCLC cell lines is selected for in vivo experiments in nude mice. Previously, the inventors showed an inhibitory effect of gefitinib alone on NSCLC xenografts growth (see above). The combination of gefitinib with one of the above-evaluated interventions is tested in athymic nude mice bearing human NSCLC xenografts. E-cadherin inducible cell lines from the in vitro experiments are inoculated subcutaneously in nude mice. Mice are treated with gefitinib with and without the agent that improved the gefitinib sensitivity. The two groups are evaluated for differences in tumor growth inhibition. Expression of E-cadherin, SIP1 and ZEB1 are evaluated both prior to and post-treatment by real-time RT-PCR and immunohistochemistry. ZEB1 antibody (Santa Cruz, Calif.) and SIP1 antibody (a gift from Dr. van Grunsven) are used in the immunohistochemistry. However, new antibodies can readily be generated if the above antibodies are not effective at detecting proteins in the IHC assays.

The results of these experiments dissect out the events leading to gefitinib resistance in order to develop treatment modifications that bypass resistance.

### Example 5

The following example describes the identification of a biomarker panel that discriminates lapatinib-sensitive colon cancer cell lines from lapatinib-resistant colon cancer cell lines. A colon cancer cell line is evaluated for genes that discriminate between lapatinib-sensitive and lapatinib-resistant cell lines. Lapatinib sensitivity is determined in multiple established colon cancer cell lines that are classified as either lapatinib sensitive or lapatinib-resistant. Oligonucleotide gene arrays (Affymetrix® Human Genome U133 set, 39,000 genes) are done on the cell lines. At least three distinct filtration and normalization algorithms to process the expression data are used, and a list of expressed genes is generated. This approach is used in combination with at least five distinct machine learning algorithms to identify and build a test set of sensitivity-predictor genes. The identified sensitivity-predictor genes are selected for RT-PCR testing/screening and verified via RT-PCR for discrimination between lapatinib-sensitive and lapatinib-resistant cell lines.

### Example 6

The following example describes the identification of a biomarker panel that discriminates gefitinib-sensitive breast cancer cell lines from gefitinib-resistant breast cancer cell lines. A breast cancer cell line is evaluated for genes that discriminate between gefitinib-sensitive and gefitinib-resistant cell lines. Gefitinib sensitivity is determined in multiple established breast cancer cell lines that are classified as either gefitinib sensitive or gefitinib-resistant. Oligonucleotide gene arrays (Affymetrix® Human Genome U133 set, 39,000 genes) are done on the cell lines. At least three distinct filtration and normalization algorithms to process the expression data are used, and a list of expressed genes is generated. This approach is used in combination with at least five distinct machine learning algorithms to identify and build a test set of sensitivity-predictor genes. The identified sensitivity-predictor genes are selected for RT-PCR testing/screening and verified via RT-PCR for discrimination between gefitinib-sensitive and gefitinib-resistant cell lines.

### Example 7

The following example describes the identification of a biomarker panel that discriminates erlotinib-sensitive non-small cell lung cancer cell lines from erlotinib-resistant non-small cell lung cancer cell lines. A non-small cell lung cancer cell line is evaluated for genes that discriminate between erlotinib-sensitive and erlotinib-resistant cell lines. Erlotinib sensitivity is determined in multiple established non-small cell lung cancer cell lines that are classified as either erlotinib-sensitive or erlotinib-resistant. Oligonucleotide gene arrays (Affymetrix® Human Genome U133 set, 39,000 genes) are done on the cell lines. At least three distinct filtration and normalization algorithms to process the expression data are used, and a list of expressed genes is generated. This approach is used in combination with at least five distinct machine learning algorithms to identify and build a test set of sensitivity-predictor genes. The identified sensitivity-predictor genes are selected for RT-PCR testing/screening and verified via RT-PCR for discrimination between erlotinib-sensitive and erlotinib-resistant cell lines.

### Example 8

The following example describes the identification of a biomarker panel that discriminates erlotinib-sensitive breast cancer cell lines from erlotinib-resistant breast cancer cell lines. A breast cancer cell line is evaluated for genes that discriminate between erlotinib-sensitive and resistant cell lines. Erlotinib sensitivity is determined in multiple established breast cancer cell lines that are classified as either erlotinib sensitive or erlotinib-resistant. Oligonucleotide gene arrays (Affymetrix® Human Genome U133 set, 39,000 genes) are done on the cell lines. At least three distinct filtration and normalization algorithms to process the expression data are used, and a list of expressed genes is generated. This approach is used in combination with at least five distinct machine learning algorithms to identify and build a test set of sensitivity-predictor genes. The identified sensitivity-predictor genes are selected for RT-PCR testing/screening and verified via RT-PCR for discrimination between erlotinib-sensitive and erlotinib-resistant cell lines.

### Example 9

The following example describes the identification of a biomarker panel that discriminates lapatinib-sensitive colorectal cancer cell lines from lapatinib-resistant colorectal cancer cell lines. A colorectal cancer (CRC) cell line is evaluated for genes that discriminate between lapatinib-sensitive and lapatinib-resistant cell lines. Lapatinib sensitivity is determined in multiple established CRC cancer cell lines that are classified as either lapatinib sensitive or lapatinib-resistant. Oligonucleotide gene arrays (Affymetrix® Human Genome U133 set, 39,000 genes) are performed on the CRC cell lines. At least three distinct filtration and normalization algorithms to process the expression data are used, and a list of expressed genes is generated. This approach is used in combination with at least five distinct machine learning algorithms to identify and build a test set of sensitivity-predictor genes. The identified sensitivity-predictor genes are selected for RT-PCR testing/screening and verified via RT-PCR for discrimination between lapatinib-sensitive and lapatinib-resistant CRC cell lines.

### Example 10

The following example describes the identification of a biomarker panel that discriminates erlotinib-sensitive pancreatic cancer cell lines from erlotinib-resistant pancreatic cancer cell lines. A pancreatic cancer cell line is evaluated for genes that discriminate between erlotinib-sensitive and erlotinib-resistant cell lines. Erlotinib sensitivity is determined in multiple established pancreatic cancer cell lines that are classified as either erlotinib-sensitive or erlotinib-resistant. Oligonucleotide gene arrays (Affymetrix® Human Genome U133 set, 39,000 genes) are performed on the pancreatic cancer cell lines. At least three distinct filtration and normalization algorithms to process the expression data are used, and a list of expressed genes is generated. This approach is used in combination with at least five distinct machine learning algorithms to identify and build a test set of sensitivity-predictor genes. The identified sensitivity-predictor genes are selected for RT-PCR testing/screening and verified via RT-PCR for discrimination between erlotinib-sensitive and erlotinib-resistant pancreatic cell lines. (Buck et al., Mol. Cancer. Ther., 5(8): 2051-2059 (2006)).

### Example 11

In this example, a method to identify and correlate specific gene expression products in breast cancer that predict responsiveness to erlotinib. Breast cancer cell lines are treated with varying dosages of erlotinib, including the recommended and established ranges for the commercially available product. EGFR, Her2, ErbB3, Her3, and E-cadherin cell surface expression on the established breast cancer cell lines is evaluated by flow cytometry with antibodies specific to each cell-surface marker. After determining the cell-surface expression levels of the identified markers, the presence of phosphorylated forms for each marker are assayed via western blotting. Detection of phosphorylated proteins is achieved via the use of commercial/established antibodies for such molecules. Finally, inhibition of growth in the breast cancer cell lines following treatment with erlotinib is determined by MTT assay using established methods and detection techniques known in the art. The correlation of marker expression, marker phosphorylation, and growth inhibition in response to erlotinib treatment can establish which markers are predictive of erlotinib sensitivity.

### Example 12

In this example, a method to identify and correlate specific gene expression products in colorectal cancer that predict responsiveness to lapatinib. Colorectal cancer (CRC) cell lines are treated with varying dosages of lapatinib, including the recommended and established ranges for the commercially available product. EGFR, Her2, ErbB3, Her3, and E-cadherin cell surface expression on the established CRC cancer cell lines is evaluated by flow cytometry with antibodies specific to each cell-surface marker. After determining the cell-surface expression levels of the identified markers, the presence of phosphorylated forms for each marker are assayed via western blotting. Detection of phosphorylated proteins is achieved via the use of commercial/established antibodies for such molecules. Finally, inhibition of growth in the CRC cancer cell lines following treatment with lapatinib is determined by MTT assay using established methods and detection techniques known in the art. The correlation of marker expression, marker phosphorylation, and growth inhibition in response to lapatinib treatment can establish which markers are predictive of lapatinib sensitivity.

### Example 13

In this example, a method to identify and correlate specific gene expression products in breast cancer that predict responsiveness to gefitinib. Breast cancer cell lines are treated with varying dosages of gefitinib, including the recommended and established ranges for the commercially available product. EGFR, Her2, ErbB3, Her3, and E-cadherin cell surface expression on the established breast cancer cell lines is evaluated by flow cytometry with antibodies specific to each cell-surface marker. After determining the cell-surface expression levels of the identified markers, the presence of phosphorylated forms for each marker are assayed via western blotting. Detection of phosphorylated proteins is achieved via the use of commercial/established antibodies for such molecules. Finally, inhibition of growth in the breast cancer cell lines following treatment with gefitinib is determined by MTT assay using established methods and detection techniques known in the art. The correlation of marker expression, marker phosphorylation, and growth inhibition in response to gefitinib treatment can establish which markers are predictive of gefitinib sensitivity.

### Example 14

In this example, a method to identify and correlate specific gene expression products in non-small cell lung cancer that predict responsiveness to erlotinib. Non-small cell lung cancer cell lines are treated with varying dosages of erlotinib, including the recommended and established ranges for the commercially available product. EGFR, Her2, ErbB3, Her3, and E-cadherin cell surface expression on the established non-small cell lung cancer cell lines is evaluated by flow cytometry with antibodies specific to each cell-surface marker. After determining the cell-surface expression levels of the identified markers, the presence of phosphorylated forms for each marker are assayed via western blotting. Detection of phosphorylated proteins is achieved via the use of commercial/established antibodies for such molecules. Finally, inhibition of growth in the non-small cell lung cancer cell lines following treatment with erlotinib is determined by MTT assay using established methods and detection techniques known in the art. The correlation of marker expression, marker phosphorylation, and growth inhibition in response to erlotinib treatment can establish which markers are predictive of erlotinib sensitivity.

### Example 15

In this example, a method to identify and correlate specific gene expression products in pancreatic cancer that predict responsiveness to erlotinib. Pancreatic cancer cell lines are treated with varying dosages of erlotinib, including the recommended and established ranges for the commercially available product. EGFR, Her2, ErbB3, Her3, and E-cadherin cell surface expression on the established pancreatic cancer cell lines is evaluated by flow cytometry with antibodies specific to each cell-surface marker. After determining the cell-surface expression levels of the identified markers, the presence of phosphorylated forms for each marker are assayed via western blotting. Detection of phosphorylated proteins is achieved via the use of commercial/established antibodies for such molecules. Finally, inhibition of growth in the pancreatic cancer cell lines following treatment with erlotinib is determined by MTT assay using established methods and detection techniques known in the art. The correlation of marker expression, marker phosphorylation, and growth inhibition in response to erlotinib treatment can establish which markers are predictive of erlotinib sensitivity. (Buck et al., Mol. Cancer. Ther., 5(8): 2051-2059 (2006)).

### Example 16

In this example, a method to identify and correlate specific gene expression products in colorectal cancer that predict responsiveness to erlotinib. Colorectal cancer (CRC) cell lines are treated with varying dosages of erlotinib, including the recommended and established ranges for the commercially available product. EGFR, Her2, ErbB3, Her3, and E-cadherin cell surface expression on the established CRC cancer cell lines is evaluated by flow cytometry with antibodies specific to each cell-surface marker. After determining the cell-surface expression levels of the identified markers, the presence of phosphorylated forms for each marker are assayed via western blotting. Detection of phosphorylated proteins is achieved via the use of commercial/established antibodies for such molecules. Finally, inhibition of growth in the CRC cancer cell lines following treatment with erlotinib is determined by MTT assay using established methods and detection techniques known in the art. The correlation of marker expression, marker phosphorylation, and growth inhibition in response to lapatinib treatment can establish which markers are predictive of erlotinib sensitivity. (Buck et al., Mol. Cancer. Ther., 5(8): 2051-2059 (2006)).

### Example 17

In this example, a method to identify and correlate specific gene expression products in urothelial cancer that predict responsiveness to cetuximab. Urothelial carcinoma cell lines are treated with varying dosages of cetuximab, including the recommended and established ranges for the commercially available product. EGFR, Her2, ErbB3, Her3, and E-cadherin cell surface expression on the established CRC cancer cell lines is evaluated by flow cytometry with antibodies specific to each cell-surface marker. After determining the cell-surface expression levels of the identified markers, the presence of phosphorylated and unphosphorylated forms for each marker are assayed via western blotting. Detection of phosphorylated proteins is achieved via the use of commercial/established antibodies for such molecules. Finally, inhibition of growth in the urothelial carcinoma cancer cell lines following treatment with cetuximab is determined by MTT assay using established methods and detection techniques known in the art. The correlation of marker expression, marker phosphorylation, and growth inhibition in response to lapatinib treatment can establish which markers are predictive of erlotinib sensitivity. (Black et al., Clin. Cancer Res. 14(5): 1478-1486 (2008)).

### Example 18

In this example, a method to identify and correlate specific gene expression products in head and neck cancer that predict responsiveness to gefitinib. Head and neck cancer cell lines are treated with varying dosages of gefitinib, including the recommended and established ranges for the commercially available product. EGFR, Her2, ErbB3, Her3, and E-cadherin cell surface expression on the established head and neck cancer cell lines is evaluated by flow cytometry with antibodies specific to each cell-surface marker. After determining the cell-surface expression levels of the identified markers, the presence of phosphorylated forms for each marker are assayed via western blotting. Detection of phosphorylated proteins is achieved via the use of commercial/established antibodies for such molecules. Finally, inhibition of growth in the head and neck cancer cell lines following treatment with gefitinib is determined by MTT assay using established methods and detection techniques known in the art. The correlation of marker expression, marker phosphorylation, and growth inhibition in response to gefitinib treatment can establish which markers are predictive of gefitinib sensitivity. (Frederick et al., Mol. Cancer. Ther., 6(6): 1683-1691 (2007)).

### Example 19

### Expression of E-Cadherin, Zeb-1 and EGFR in Pancreatic Cancer and Head & Neck Cancer (H&N) Cell Lines

Pancreatic and H&N cancer cell lines were assayed for the expression of E cadherin, EGFR, and ZEB1. The expression was assayed using RT-PCR with forward and reverse primers specific for the respective genes. A control of β-actin was also assayed and shown in the results. E-cadherin, EGFR and ZEB1 expression levels are described relative to the β-actin expression levels.

Total RNA was prepared from cell lines using the RNAeasy kit (Qiagen) and RNA isolation from paraffin embedded tissue kit (qiagen). cDNA was transcribed from 1 μg of each sample using AffinityScript™ QPCR cDNA Synthesis Kit (Stratagene, La Jolla, Calif.). Quantitative Real-Time PCR were performed 1/20th of the cDNA reaction using the Brilliant® SYBR® Green QPCR Core Reagent Kit (Stratagene). Amplification data were analyzed by using GENEAMP 5700 SDS software, converted into cycle numbers at a set cycle threshold (Ct values) and quantified in relation to a standard. Human adult-lung RNA (Clontech Lab. Inc) was used as standard at 20, 4, 0.8, 0.16 ng in all the experiments. To normalize for the amount of input cDNA, the quantified relative amount of the generated product was divided by the amount generated for 13-Actin. Cycling conditions were 50° C. for 10 seconds and 95° C. for 10 minutes, followed by 46 cycles at 95° C. for 15 seconds and 60° C. for 1 minute. All samples were performed in triplicates.

Pancreatic cell-lines assayed included Panc04.03, Panc02.03, HS766T, Panc03.27 and HPAFII. Head & Neck cancer cell lines assayed included Detroit562, FaDu, UMSCC19, and UMSCC22B. In both pancreatic and H&N cell lines there was negative correlation between expression of Zeb-1 and E-cadherin.

| | SQ Mean/β actin | | |
|---|---|---|---|
| Cell lines | Ecad | EGFR | ZEB1 |
| Panc04.03 | 1.866 | 2.306 | 0.017 |
| Panc02.03 | 3.689 | 5.294 | 0.022 |
| HS766T | 0.004 | 1.121 | 0.915 |
| Panc03.27 | 1.415 | 2.623 | 0.057 |
| HPAFII | 2.729 | 2.401 | 0.048 |
| Detroit562 | 1.053 | 3.654 | 0.013 |
| FaDu | 1.273 | 4.662 | 0.024 |
| UMSCC19 | 1.295 | 1.577 | 0.021 |
| UMSCC22B | 0.001 | 1.055 | 0.485 |

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A diagnostic method comprising:
a) providing a sample of cancer cells of epithelial origin from a patient to be tested;
b) detecting in the sample the expression of at least one gene chosen from a panel of genes whose expression has been correlated with sensitivity or resistance to an antibody that binds EGFR, wherein the at least one gene is chosen from one or more genes selected from the group consisting of E-cadherin, RAB25, integrin beta 6 (ITGB6), vimentin, ZEB1 and SIP1; and
c) comparing the level of expression of at least one gene detected in the patient sample to a level of expression of at least one gene that has been correlated with sensitivity or resistance to the antibody that binds EGFR.

2. The diagnostic method of claim 1, comprising detecting expression of E-cadherin.

3. The diagnostic method of claim 1, comprising detecting expression of RAB25.

4. The diagnostic method claim 1, comprising detecting expression of integrin beta 6 (ITGB6).

5. The diagnostic method of claim 1, comprising detecting expression of vimentin.

6. The diagnostic method of claim 1, comprising detecting expression of ZEB1.

7. The diagnostic method of claim 1, comprising detecting expression of SIP1.

8. The diagnostic method of claim 1, wherein the antibody is cetuximab, panitumumab, nimotuzumab, or matuzumab.

9. The diagnostic method of claim 8, wherein the antibody is cetuximab.

10. The diagnostic method of claim 8, wherein the antibody is panitumumab.

11. The diagnostic method of claim 8, wherein the antibody is nimotuzumab.

12. The diagnostic method of claim 8, wherein the antibody is matuzumab.

13. The diagnostic method of claim 1, wherein the cancer cells are selected from breast cancer cells, skin cancer cells, bladder cancer cells, colon cancer cells, prostate cancer cells, uterine cancer cells, cervical cancer cells, ovarian cancer cells, esophageal cancer cells, stomach cancer cells, gastrointestinal cancer cells, pancreatic cancer cells, laryngeal cancer cells, and lung cancer cells.

14. The diagnostic method of claim 13 where the cancer cells are from pancreatic cancer cells.

15. The diagnostic method of claim 13 where the cancer cells are from head and neck cancer cells.

16. The diagnostic method of claim 13 where the cancer cells are from breast cancer cells.

17. The diagnostic method of claim 13 where the cancer cells are from colon cancer cells.

18. The diagnostic method of claim 1 further comprising: d) selecting the patient as being predicted to benefit from therapeutic administration of the antibody that binds EGFR.

19. The diagnostic method of claim 18, wherein the expression of at least one gene in the patient's cancer cells is statistically more similar to the expression levels of at least one gene that has been correlated with sensitivity to the antibody that binds EGFR than to resistance to the antibody that binds EGFR.

20. The diagnostic method of claim 18, wherein the expression of at least one gene in the patient's cancer cells is statistically more similar to the expression levels of at least one gene that has been correlated with resistance to the antibody that binds EGFR than to resistance to the antibody that binds EGFR.

21. The diagnostic method of claim 1, wherein the panel of genes in (b) is identified by a method comprising:
a) providing a sample of cells that are sensitive or resistant to treatment with the antibody that binds EGFR;
b) detecting the expression of at least one gene in the antibody-sensitive cells as compared to the level of expression of the gene or genes in the antibody-resistant cells; and
c) identifying a gene or genes having a level of expression in the antibody-sensitive cells that is statistically significantly different than the level of expression of the gene or genes in the antibody-resistant cells.

22. The diagnostic method of claim 1, wherein expression of the gene(s) is detected by a method selected from the group of:
(i) measuring amounts of transcripts of the gene in the tumor cells;
(ii) detecting hybridization of at least a portion of the gene or a transcript thereof to a nucleic acid molecule comprising a portion of the gene or a transcript thereof in a nucleic acid array; and
(iii) detecting the production of a protein encoded by the gene.

23. A method of detecting sensitivity of an epithelial-origin cancer to an antibody the binds EGFR comprising:
a) detecting in a sample of tumor cells from a patient to be tested, the expression of one or more genes selected from the group consisting of E-cadherin, RAB25, integrin beta 6 (ITGB6), vimentin, ZEB1 and SIP1;
b) comparing the level of expression of the one or more genes detected in the patient sample to a gene expression level of E-cadherin, RAB25, integrin beta 6 (ITGB6), vimentin, ZEB1 or SIP1 that has been correlated with sensitivity or resistance to an antibody that binds EGFR; and
c) identifying the expression level of the one or more genes detected in the patient sample that are statistically more similar to the expression level of E-cadherin, RAB25, integrin beta 6 (ITGB6), vimentin, ZEB1 or SIP1 that has been correlated with sensitivity than to the expression levels that have been correlated with resistance.

24. The method of claim 23 wherein the gene is E-cadherin.

25. The method of claim 23 where the gene is RAB25.

26. The method of claim 23 where the gene is integrin beta 6 (ITGB6).

27. The method of claim 23 wherein the gene is vimentin.

28. The method of claim 23 wherein the gene is ZEB1.

29. The method of claim 23 wherein the gene is SIP1.

30. The method of claim 23 wherein the antibody is cetuximab, panitumumab, nimotuzumab or matuzumab.

31. The method of claim 30 wherein the antibody is cetuximab.

32. The method of claim 30 wherein the antibody is panitumumab.

33. The method of claim 30 wherein the antibody is nimotuzumab.

34. The method of claim 30 wherein the antibody is matuzumab.

35. The method of claim 23 wherein the cancer is breast cancer, skin cancer, bladder cancer, pancreatic cancer, colon cancer, gastro-intestinal cancer, prostate cancer, uterine cancer, cervical cancer, ovarian cancer, esophageal cancer, stomach cancer, head and neck cancer, laryngeal cancer, or lung cancer.

36. The method of claim 35 where the cancer is breast cancer.

37. The method of claim 35, where the cancer is colon cancer.

38. The method of claim 35, where the cancer is pancreatic cancer.

39. The method of claim 35, where the cancer is head and neck cancer.

40. A kit comprising reagents for the detection of expression levels that have been correlated with sensitivity or resistance to an EGFR inhibitor of one or more genes selected from E-cadherin, RAB25, integrin beta 6, vimentin, ZEB1 and SIP1 in a sample of cancer cells.

41. The kit of claim 40, further comprising a compilation comprising E-cadherin, RAB25, integrin beta 6, vimentin, ZEB1 or SIP1 expression levels that have been correlated with sensitivity or resistance to an EGFR inhibitor.

42. The kit of claim 41 wherein the gene is E-cadherin.

43. The kit of claim 41 wherein the gene is ZEB1.

44. The kit of claim 41 wherein the gene is SIP1.

45. The kit of claim 41 wherein the gene is RAB25.

46. The kit of claim 41 wherein the gene is integrin beta 6.

47. The kit of claim 41 wherein the gene is vimentin.

48. A method of treating cancer in a patient comprising:
a) detecting the expression levels of one or more genes selected from E-cadherin, RAB25, integrin beta 6, vimentin, ZEB1 and SIP1; and
b) administering an EGFR inhibitor.

49. The method of claim 48, wherein the gene is E-cadherin.

50. The method of claim 48, wherein the gene is RAB25.

51. The method of claim 48, wherein the gene is integrin beta 6.

52. The method of claim 48, wherein the gene is vimetin.

53. The method of claim 48, wherein the gene is ZEB1.

54. The method of claim 48, wherein the gene is SIP1.

55. The method of claim 48, wherein the EGFR inhibitor is selected from gefitinib, erlotinib, imatinib, lapatinib, and semazinib.

56. The method of claim 55, wherein the EGFR inhibitor is gefitinib.

57. The method of claim 55, wherein the EGFR inhibitor is erlotinib.

58. The method of claim 55, wherein the EGFR inhibitor is imatinib.

59. The method of claim 55, wherein the EGFR inhibitor is lapatinib.

60. The method of claim 55, wherein the EGFR inhibitor is semazinib.

61. The method of claim 48, wherein the EGFR inhibitor is selected from cetuximab, panitumumab, nimotuzumab, and metuzumab.

62. The method of claim 61, wherein the EGFR inhibitor is cetuximab.

63. The method of claim 61, wherein the EGFR inhibitor is panitumumab.

64. The method of claim 61, wherein the EGFR inhibitor is nimotuzumab.

65. The method of claim 61, wherein the EGFR inhibitor is metuzumab.

66. A method of treating cancer in a patient comprising:
a) upregulating E-cadherin in a cancer cell by administering to the patient at least one ZEB1 inhibitor; and
b) administering to the patient an EGFR inhibitor.

67. A method of treating cancer in a patient comprising:
a) upregulating E-cadherin in a cancer cell by administering to the patient at least one SIP1 inhibitor; and
b) administering to the patient an EGFR inhibitor.

68. The diagnostic method of claim 1 wherein the at least one gene or genes comprises one or more genes selected from the group consisting of E-cadherin (SEQ ID NO: 3), RAB25 (SEQ ID NO: 83), integrin beta 6 (ITGB6) (SEQ ID NO: 137), integrin beta 6 (ITGB6) (SEQ ID NO: 52), ZEB1 (SEQ ID NO:196), SIP1 (SEQ ID NO: 197), and vimentin (SEQ ID NO: 195).

69. The diagnostic method of claim 68, comprising detecting the expression of E-cadherin (SEQ ID NO: 3).

70. The diagnostic method of claim 68, comprising detecting the expression of RAB25 (SEQ ID NO: 83).

71. The diagnostic method of claim 68, comprising detecting the expression of integrin beta 6 (ITGB6) (SEQ ID NO: 137).

72. The diagnostic method of claim 68, comprising detecting the expression of integrin beta 6 (ITGB6) (SEQ ID NO: 52).

73. The diagnostic method of claim 68, comprising detecting the expression of vimentin (SEQ ID NO: 195).

74. The method of claim 23 wherein the one or more genes is selected from the group consisting of E-cadherin (SEQ ID NO: 3), RAB25 (SEQ ID NO: 83), integrin beta 6 (ITGB6) (SEQ ID NO: 137), integrin beta 6 (ITGB6) (SEQ ID NO: 52), ZEB1 (SEQ ID NO: 196), SIP1 (SEQ ID NO: 197), and vimentin (SEQ ID NO: 195).

75. The diagnostic method of claim 74, comprising detecting the expression of E-cadherin (SEQ ID NO: 3).

76. The diagnostic method of claim 74, comprising detecting the expression of RAB25 (SEQ ID NO: 83).

77. The diagnostic method of claim 74, comprising detecting the expression of integrin beta 6 (ITGB6) (SEQ ID NO: 137).

78. The diagnostic method of claim 74, comprising detecting the expression of integrin beta 6 (ITGB6) (SEQ ID NO: 52).

79. The diagnostic method of claim 74, comprising detecting the expression of vimentin (SEQ ID NO: 195).

80. The kit of claim 40, wherein the one or more genes is selected from E-cadherin (SEQ ID NO: 3), RAB25 (SEQ ID NO: 83), integrin beta 6 (ITGB6) (SEQ ID NO: 137), integrin beta 6 (ITGB6) (SEQ ID NO: 52), ZEB1 (SEQ ID NO:196), SIP1 (SEQ ID NO: 197), and vimentin (SEQ ID NO: 195).

81. The kit of claim 80, wherein the gene is E-cadherin (SEQ ID NO: 3).

82. The kit of claim 80, wherein the gene is RAB25 (SEQ ID NO: 83).

83. The kit of claim 80, wherein the gene is integrin beta 6 (ITGB6) (SEQ ID NO: 137).

84. The kit of claim 80, wherein the gene is integrin beta 6 (ITGB6) (SEQ ID NO: 52).

85. The kit of claim 80, wherein the gene is vimentin (SEQ ID NO: 195).

86. The method of claim 48, wherein the one or more genes is selected from E-cadherin (SEQ ID NO: 3), RAB25 (SEQ ID NO: 83), integrin beta 6 (ITGB6) (SEQ ID NO: 137), integrin beta 6 (ITGB6) (SEQ ID NO: 52), ZEB1 (SEQ ID NO: 196), SIP1 (SEQ ID NO: 197), and vimentin (SEQ ID NO: 195).

87. The method of claim 86, wherein the gene is E-cadherin (SEQ ID NO: 3).

88. The method of claim 86, wherein the gene is RAB25 (SEQ ID NO: 83).

89. The method of claim 86, wherein the gene is integrin beta 6 (ITGB6) (SEQ ID NO: 137).

90. The method of claim 86, wherein the gene is integrin beta 6 (ITGB6) (SEQ ID NO: 52).

91. The method of claim 86, wherein the gene is vimentin (SEQ ID NO: 195).

92. The method of claim 68, wherein the gene is ZEB1 (SEQ ID NO: 196).

93. The method of claim 68, wherein the gene is SIP1 (SEQ ID NO: 197).
